(19) United States
(12) Patent Application Publication
(10) Pub. No.: US 2025/0228896 A1
(43) Pub. Date: Jul. 17, 2025

(54) MATRIX BOUND NANOVESICLES ENCAPSULATED IN HYDROGELS

(71) Applicant: University of Pittsburgh - Of the Commonwealth System of Higher Education, Pittsburgh, PA (US)

(72) Inventors: Stephen Francis Badylak, Lafayette, IN (US); George S. Hussey, Cranberry Township, PA (US)

(73) Assignee: University of Pittsburgh - Of the Commonwealth System of Higher Education, Pittsburgh, PA (US)

(21) Appl. No.: 18/853,393

(22) PCT Filed: Apr. 7, 2023
(86) PCT No.: PCT/US2023/065525

§ 371 Date: Oct. 1, 2024

Related U.S. Application Data
(60) Provisional application No. 63/329,319, filed on Apr. 8, 2022.

(51) Int. Cl. A61K 35/22; A61K 9/00; A61K 9/06; A61K 9/50; A61K 35/36; A61K 38/48; A61P 1/04

(52) U.S. Cl. CPC A61K 35/22; A61K 9/0031; A61K 9/06; A61K 9/5068; A61K 35/36; A61K 38/4826; A61K 38/488; A61P 1/04; C12Y 304/21004; C12Y 304/23

(57) ABSTRACT

Compositions are disclosed herein that include an extracellular matrix (ECM) hydrogel and matrix bound nanovesicles (MBV). These compositions provide a synergistic effect and are of use for treating subjects.

### CROSS-REFERENCE TO RELATED APPLICATIONS

This application claims priority to and the benefit of U.S. Provisional Patent Application No. 63/329,319 filed on Apr. 8, 2022, the contents of which are incorporated by reference herein in their entirety.

### FIELD OF THE DISCLOSURE

This is related to the field of extracellular matrix (ECM) compositions, specifically to compositions that include both an ECM hydrogel and matrix bound nanovesicles (MBV), and the use of these compositions.

### REFERENCE TO AN ELECTRONIC SEQUENCE LISTING

The contents of the electronic sequence listing (Sequences.xml; Size: 3,955 bytes; and Date of Creation: Apr. 6, 2023) is herein incorporated by reference in its entirety.

### BACKGROUND

Hydrogels composed of purified ECM components such as collagen, hyaluronic acid, silk fibroin, laminin, and fibronectin, have been widely used in tissue engineering applications. However, these purified, single component ECM biomaterials lack the complex biochemistry of native tissue ECM. Decellularization of whole tissues or organs provides for an alternative method for harvesting ECM that preserves the biochemistry of native tissue ECM. A major advancement in the use of decellularized ECM is the ability to form hydrogels, thereby expanding the clinical applicability of decellularized ECM. However, a need remains for enhancing the activities of ECM hydrogels.

### SUMMARY OF THE DISCLOSURE

Compositions are disclosed herein that include an ECM hydrogel and MBV. These compositions provide a synergistic effect, such as for decreasing inflammation.

In some aspects, compositions are disclosed that include an ECM hydrogel including a) solubilized extracellular matrix and b) exogenous MBV derived from extracellular matrix, wherein the MBV do not express CD63 and CD81 or are CD63^{lo}CD81^{lo} and wherein the MBV do not contain alkaline phosphatase. The exogenous MBV are present in the ECM hydrogel at a concentration of at least about 1×10⁵ to 1×10²⁰ particles/mL. The composition may be: i) sheer thinning; ii) have a storage modulus (G′) of about 50 Pa to about 200 Pa, and a loss modulus (G″) of about 5 Pa to about 20 Pa, and a G′ to G″ ratio of about 4:1 to about 15:1 at 37° C., and iii) have a 50% degradation rate of 24 hours to 14 days.

In other aspects, compositions are disclosed that include an acidic solution comprising an exogenous acid protease and solubilized extracellular matrix, e.g., intact ECM; and exogenous MBV derived from extracellular matrix, wherein the MBV do not express CD63 and CD81 or are CD63^{lo}CD81^{lo} and wherein the MBV do not contain alkaline phosphatase. The exogenous MBV are present in the composition at a concentration of at least about 1×10⁵ to 1×10²⁰ particles/mL, and the acidic solution, when neutralized to a pH of between about 7.0 to about 7.8 forms a gel at a temperature greater than 25° C.

In more aspects, compositions are disclosed that include solubilized extracellular matrix, e.g., intact ECM; a deactivated or inactivated exogenous acid protease; and exogenous MBV derived from extracellular matrix. The MBV do not express CD63 and CD81 or are CD63^{lo}CD81^{lo} and do not contain alkaline phosphatase. These exogenous MBV are present in the composition at a concentration of at least about 1×10⁵ to 1×10²⁰ particles/mL. The composition enters the liquid phase at a temperature less than 25° C. and enters a gel phase at a temperature greater than 25° C., and has a pH of between about 7 and about 7.8. The ECM may have been solubilized by an acid protease.

The foregoing and other features and advantages of the invention will become more apparent from the following detailed description of several aspects which proceeds with reference to the accompanying figures.

### BRIEF DESCRIPTION OF THE FIGURES

FIG. 1 provides a schematic overview of the study in which ulcerative colitis was induced in study animals and a treatment regimen provided. Animals were given 5.5% DSS water for 6 days and then given normal water through day 4. Treatments occurred from Day 0 to Day 4 which were daily enemas or injections on days 0 and 2. Colonic tissue was explanted from test animals on day 4.

FIGS. 2A-2C is a series of photographs showing an exemplary enema delivery preparation and procedure. FIG. 2A shows a SURFLO® winged infusion catheter used in performing an enema and cutting location to remove the needle. FIG. 2B shows the needle was cut off of the catheter and two marks (black arrows) were made, one at 8 cm from the end to ensure consistency of depth between enemas and the other at 4 cm to ensure even withdrawal of the catheter as material was injected. FIG. 2C is a digital image showing how the catheter was inserted through the anus and into the colon of a test animals and the 5 mL enema material was slowly delivered.

FIG. 3 provides graphs of food consumption (right) and water consumption (left) as measured for each animal daily during the study period.

FIG. 4. is a graph showing the fraction of initial weight for the animals over the study period. The weight of each animal was measured daily and normalized to the initial weight of the animal at day-6.

FIG. 5 provides a graph of fractional stool consistency scores (left) and fractional stool blood content (right) as scored for each animal daily. Stool blood content was determined using visual observation as well as COLOSCREEN® ES Lab Pack Fecal Occult tests and scored [0=no blood, 2=occult blood, 4=gross bleeding]. Stool consistency was scored [0=normal, 2=loose, 4=diarrhea]. Fractional scores were determined by normalizing individual rat scores to the scores given at Day 0.

FIG. 6 provides bar graphs of the colon length (left) and gross anatomic scoring of colon explants (right). For the bar graph on the left, length of each colon from the cecum to the rectum were measured following explant. All groups given DSS appeared to have a shorter colon than the healthy group. For the bar graph on the right, explanted colons were scored by blinded investigators for macroscopic evidence of ulceration and inflammation. All treatment groups showed lower scores (less disease) than the diseased control.

FIGS. 7A-7G provide images of histologic analysis of H&E stained rat colonic tissue sections from test animals, with the images in FIGS. 7A-F providing a magnified view of sections of the larger tissue samples shown in FIG. 7G. FIGS. 7A and 7G (i) are images from a healthy animals. FIGS. 7B and 7G (ii) are images from a diseased animal. FIGS. 7C and 7G (vi) are images from a diseased animal receiving MBV in a saline enema. FIGS. 7D and 7G (iii) are images from a diseased animal receiving an ECM hydrogel enema. FIGS. 7E and 7G (v) are images from a diseased animal receiving an MBV-infused ECM hydrogel enema. FIGS. 7F and 7G (iv) are images from a diseased animal receiving MB by i.v. injection.

FIGS. 8A-8B provide a comparison of surface markers for exosomes, bone microvesicles (MV) and MBV. The figure shows the results of EXO-CHECK™ Exosome Antibody Arrays (System Biosciences) comparing levels of the various markers noted in murine exosomes, murine bone matrix vesicles (bone MV), and murine matrix bound nanovesicles (MBV). FIG. 8A provides digital images of the arrays and FIG. 8B is a graph showing the relative expression of each of the noted markers in the exosomes versus bone MV versus MBV. The data show that MBV are different from exosomes, bone microvesicles (MV) based on the profile of surface markers. The MBV do not express or have low expression of CD63, EpCAM, ANXA5, TSG101, GM130, FLOT1, ICAM1, ALIX, and CD81, as compared to Bone MV or exosome levels of these markers as shown in the bar graphs in the lower panel.

FIG. 9 is a western blot showing that bone MV markers Annexin V and Tissue Non-specific Alkaline Phosphatase (TNAP) are expressed by bone MV. Lysate prepared from 1711A Cells was used as a positive control. The results of this experiment show that matrix bound nanovesicles (MBV) are devoid of any expression of both markers of bone microvesicles, TNAP and Annexin V. Plasma exosomes do express Annexin V, but do not express TNAP. These results clearly distinguish MBV from both exosomes and bone microvesicles. Notably, the MBV used were isolated from muscle tissue.

FIG. 10 is a bar graph showing the different effects of macrophage activated-gene expression on exosomes, MV and MBV. MBV have a differential immunomodulatory effect, namely they increase M2 macrophages, when compared to exosomes or bone MV which do not have this effect. Bone Marrow-Derived Macrophages (BMDM) harvested from mice were untreated (M0) or treated with the following test articles for 24 hours: IFNγ+LPS to induce an M1 phenotype (M1), IL-4 to induce an M2-like phenotype (M2), Exosomes derived from plasma, bone MV derived from 17A cells, or MBV isolated from muscle. After treatment, the fold change in the expression of the indicated genes (Arg, CD206, Fixx, IL-6, INOS, and TNF) was evaluated by qPCR. FIG. 10 shows the downregulation of the pro-inflammatory markers IL-6 and TNF-α by MBV are clearly distinguished from the downregulation of the same two inflammatory mediators by exosomes and bone MV. MBV had a potent anti-inflammatory effect; whereas exosomes and bone MV did not have this effect.

FIGS. 11A-11E are higher magnification views of histological (hematoxylin and eosin) images representing each treatment group in the colon (disease: FIG. 11A; ECM hydrogel enema: FIG. 11B; MBV+PBS enema: FIG. 11C; MBV-infused ECM hydrogel enema: FIG. 11D; double MV i.v. injection: FIG. 11E). These are representative of the results for each group.

FIGS. 12A-E are bar graphs showing quantification of the M2:M1 macrophage ratio in treated samples at the various layers: colon (FIG. 12A), mucosa (FIG. 12B), muscularis (FIG. 12C), and submucosa (FIG. 12D). Quantification of CD68+ cells for each treatment is shown in FIG. 12E.

### DETAILED DESCRIPTION

MBV are an integral component of the ECM, are distinct from exosomes, and effectively redirect hyperinflammation in preclinical models (Hussey G S, et al. (2020) Lipidomics and RNA sequencing reveal a novel subpopulation of nanovesicle within extracellular matrix biomaterials. Sci Adv 6 (12): eaay4361; van der Merwe Y, et al. (2019) Matrix-bound nanovesicles prevent ischemia-induced retinal ganglion cell axon degeneration and death and preserve visual function. Sci Rep 9 (1): 3482). In some aspects, MBV contain immunomodulatory miRNA, proteins, and lipids and are rapidly taken up by macrophages, triggering signaling cascades and modulating gene expression essential for phenotype switching, a phenomenon well-studied in the context of ECM-based biomaterials (Hussey G S, et al. (2019) Matrix bound nanovesicle-associated IL-33 activates a pro-remodeling macrophage phenotype via a non-canonical, ST2-independent pathway. J Immunol Regen Med 3:26-35; Huleihel L, et al. (2017) Macrophage phenotype in response to ECM bioscaffolds. Semin Immunol 29:2-13). Furthermore, in some aspects, MBV administration results in upregulation of regulatory T cells (T_{REG}), a phenomenon previously characterized in the context of ECM-based biomaterials. MBV rapidly and effectively induce the reparative immune response in harsh environments including rheumatoid arthritis, traumatic muscle injury, ulcerative colitis, and esophageal cancer (Huleihel L, et al. (2017) Matrix-Bound Nanovesicles Recapitulate Extracellular Matrix Effects on Macrophage Phenotype. Tissue Eng Part A 23 (21-22): 1283-1294; Dziki J L, et al. (2016) Immunomodulation and Mobilization of Progenitor Cells by Extracellular Matrix Bioscaffolds for Volumetric Muscle Loss Treatment. Tissue Eng Part A 22 (19-20): 1129-1139; Keane T J, et al. (2017) Restoring Mucosal Barrier Function and Modifying Macrophage Phenotype with an Extracellular Matrix Hydrogel: Potential Therapy for Ulcerative Colitis. J Crohns Colitis 11 (3): 360-368; Saldin L T, et al. (2019) Extracellular Matrix Degradation Products Downregulate Neoplastic Esophageal Cell Phenotype. Tissue Eng Part A 25 (5-6): 487-498.)

Cytokine cargo stored within MBV support reparative and regulatory M2 macrophages and control bacterial infections and inflammation after acute lung injury (Liu Q, et al. (2019) IL-33-mediated IL-13 secretion by ST2+T_{REG} controls inflammation after lung injury. JCI Insight 4 (6)). ECM bioscaffolds are useful in a variety of clinical applications involving musculoskeletal, gastrointestinal, urogenital and CNS tissues (Badylak S F (2007) The extracellular matrix as a biologic scaffold material. Biomaterials. 28 (25): 3587-3593). ECM consists of the secreted structural and functional molecules of the resident cells of each tissue that define tissue identity. Such xenogeneic scaffolds do not elicit an adverse innate or adaptive immune response, and instead support an anti-inflammatory and reparative innate and adaptive immune response (Brown B N, et al. (2009) Macrophage phenotype and remodeling outcomes in response to biologic scaffolds with and without a cellular component. Biomaterials. 30 (8): 1482-1491). Use of these naturally occurring biomaterials is typically associated with (at least) partial restoration of functional, site-appropriate tissue; a process referred to as “constructive remodeling” (Badylak S F (2007) The extracellular matrix as a biologic scaffold material. Biomaterials. 28 (25): 3587-3593). ECM bioscaffolds, or degradation products of ECM bioscaffolds, have been shown to direct tissue repair through recruitment of an anti-inflammatory M2-like macrophage and T helper Type 2 (Th2) cell response, such a response is often associated with reduced local inflammation and constructive crosstalk with progenitor cells.

Matrix Bound Nanovesicles (MBV) activate the M2-like reparative and anti-inflammatory macrophage phenotype. Studies have shown that MBV are a distinct class of extracellular vesicle separate from exosomes found in body fluids (Hussey G S, et al. (2020) Lipidomics and RNA sequencing reveal a novel subpopulation of nanovesicle within extracellular matrix biomaterials. Sci Advances. 6 (12): eaay4361). As MBV survive even harsh tissue decellularization processes, they can play a fundamental role in tissue and organ development and homeostasis across species, as well as a regulatory role in the tissue response to injury. MBV may be derived from multiple, varied tissue sources. MBV are plentiful, can be lyophilized, are highly stable, and can be easily administered via tracheal instillation or nebulization.

MBV can recapitulate the effects of ECM on promoting a pro-remodeling macrophage phenotype. Macrophage gene and protein expression, cell surface markers, and functional capacity as determined by phagocytic activity, nitric oxide (NO) production, and antimicrobial activity observed were most representative of a regulatory/anti-inflammatory phenotype following treatment with MBV, consistent with previous reports describing the effects of ECM-based bioscaffolds on macrophage phenotype and function. (See, for example, PCT Publication No. WO 2017/151862A1). MBV have been shown to exert an immunomodulatory effect through a combination of miRNA, protein, and phospholipid cargoes. For example, compared to exosomes present in body fluids, MBV are highly enriched in pro-resolving lipid mediators activated by different phospholipases dependent on the pro-/anti-inflammatory context of the extracellular environment (Hussey G S, et al. (2020) Lipidomics and RNA sequencing reveal a novel subpopulation of nanovesicle within extracellular matrix biomaterials. Sci Adv 6 (12): eaay4361). Moreover, MBV are a rich and stable source of IL-33 that signals directs immune cells toward a reparative M2-like phenotype, while also stimulate repair and regulatory functions by T_{REG} in the damaged lung (Liu Q, et al. (2019) IL-33-mediated IL-13 secretion by ST2+T_{REG} controls inflammation after lung injury. JCI Insight 4 (6)). IL-33 delivery reduces bacterial super-infections after H1N1 infections by improving bacterial clearance (Robinson K M, et al. (2018) Novel protective mechanism for interleukin-33 at the mucosal barrier during influenza-associated bacterial superinfection. Mucosal immunology. 11 (1): 199-208). Additionally, MBV are enriched in miRNA 125b-5p, 143-3p, and 145-5p. Inhibition of these miRNAs within macrophages is associated with a gene and protein expression profile more consistent with a proinflammatory rather than an anti-inflammatory/regulatory phenotype (Huleihel L, et al. (2017) Matrix bound nanovesicles recapitulate extracellular matrix effects on macrophage phenotype. Tissue Eng Part A). Disclosed herein are compositions that include both MBV and an ECM hydrogel.

Compositions are disclosed herein that include an extracellular matrix (ECM) hydrogel and matrix bound nanovesicles (MBV). These compositions provide a synergistic effect and are of use for treating subjects.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a MBV” includes single or plural MBVs and is considered equivalent to the phrase “comprising at least one MBV.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.” It is further to be understood that any and all molecular weight or molecular mass values, or proportions, given for compositions are approximate, and are provided for descriptive purposes, unless otherwise indicated. Dates of GENBANK® Accession Nos. referred to herein are the sequences available at least as early as Apr. 5, 2021. All references, patent applications and publications, and GENBANK® Accession numbers cited herein are incorporated by reference. Unless otherwise indicated, “about” indicates within five percent. In case of conflict, the present specification, including explanations of terms, will control. In order to facilitate review of the various aspects of the disclosure, the following explanations of specific terms are provided:

Acid Protease: An enzyme that cleaves peptide bonds, wherein the enzyme has increased activity of cleaving peptide bonds in an acidic pH. For example and without limitation, acid proteases can include pepsin and trypsin.

Administration: The introduction of a composition (such as MBV or a pharmaceutical preparation that includes MBV) into a subject by a chosen route. The route can be local or systemic. For example, if the chosen route is intravenous, the composition is administered by introducing the composition into a vein of the subject. If the chosen route is local, the composition can be administered by introducing the composition directly into a tissue of the subject.

Animal: Living multi-cellular vertebrate organisms, a category that includes, for example, mammals and birds. The term “mammal” includes both human and non-human mammals. Similarly, the term “subject” includes both human and veterinary subjects.

Biocompatible: Any material, that, when implanted in a mammalian subject, does not provoke an adverse response in the subject. A biocompatible material, when introduced into an individual, is able to perform its intended function, and is not toxic or injurious to that individual, nor does it induce immunological rejection of the material in the subject.

Centrifugation: The process whereby a centrifugal force is applied to a mixture, whereby more-dense components of the mixture migrate away from the axis of the centrifuge relative to other less-dense components in the mixture. The force that is applied to the mixture is a function of the speed of the centrifuge rotor, and the radius of the spin. In most applications, the force of the spin will result in a precipitate (a pellet) to gather at the bottom of the centrifuge tube, where the remaining solution is properly called a “supernate” or “supernatant.” In other similar applications, a density-based separation or “gradient centrifugation” technique is used to isolate a particular species from a mixture that contains components that are both more dense and less dense than the desired component.

During the circular motion of a centrifuge rotor, the force that is applied is the product of the radius and the angular velocity of the spin, where the force is traditionally expressed as an acceleration relative to “g,” the standard acceleration due to gravity at the Earth's surface. The centrifugal force that is applied is termed the “relative centrifugal force” (RCF), and is expressed in multiples of “g.”

Comminute (comminution and comminuting): The process of reducing larger particles into smaller particles, including, without limitation, by grinding, blending, shredding, slicing, milling, or cutting. ECM can be comminuted while in any form, including, but not limited to, hydrated forms, frozen, air-dried, lyophilized, powdered, or sheet-form. “Comminuted ECM” includes intact collagen. Comminuted ECM has not been subjected to ultrasound or enzymatic digestion, e.g., with a protease, such as an acid protease.

Contacting: Placement in direct physical association, which can be in solid or liquid form.

Cytokine: The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor-α, interleukin (IL)-6, IL-10, IL-12, transforming growth factor, and interferon-γ.

Diagnosis: The process of identifying a disease by its signs, symptoms and results of various tests. The conclusion reached through that process is also called “a diagnosis.” Forms of diagnostic testing commonly performed include, without limitation, blood tests, medical imaging, and biopsy.

Enriched: A process whereby a component of interest, such as a nanovesicle, that is in a mixture has an increased ratio of the amount of that component to the amount of other components in that mixture after the enriching process as compared to before the enriching process.

Extracellular matrix (ECM): A complex mixture of structural and functional biomolecules and/or biomacromolecules including, but not limited to, structural proteins, specialized proteins, proteoglycans, glycosaminoglycans, and growth factors that surround and support cells within tissues and, unless otherwise indicated, is acellular. ECM preparations can be considered to be “decellularized” or “acellular,” meaning the cells have been removed from the source tissue through processes described herein and known in the art. By “ECM-derived material,” such as an “ECM-derived nanovesicle,” “Matrix bound nanovesicle,” “MBV” or “nanovesicle derived from an ECM” it is meant a nanovesicle that is prepared from a natural ECM or from an in vitro source wherein the ECM is produced by cultured cells. “Intact Extracellular Matrix” and “intact ECM” refers to an extracellular matrix that retains activity of its structural and non-structural biomolecules, including, but not limited to, collagens, elastins, laminins, glycosaminoglycans, proteoglycans, antimicrobials, chemoattractants, cytokines, and growth factors, such as, without limitation, comminuted ECM as described herein. The activity of the biomolecules within the ECM can be removed chemically or mechanically, for example, by cross-linking and/or by dialyzing the ECM. Intact ECM essentially has not been cross-linked and/or dialyzed, meaning that the ECM has not been subjected to a dialysis and/or a cross-linking process, or conditions other than processes that occur naturally during storage and handling of ECM prior to solubilization in making an enzymatic ECM hydrogel. Thus, ECM that is substantially cross-linked and/or dialyzed (in anything but a trivial manner which does not substantially affect the gelation and functional characteristics of the ECM in its uses described herein) is not considered to be “intact”.

Exogenous: Originating from a different source. Exogenous MBVs are produced separately, e.g., extracted from an ECM source, and added to an ECM hydrogel, that may or may not have endogenous MBV present in the ECM hydrogel. Exogenous MBV can be derived from the same tissue, or a different tissue, than the ECM used to make an ECM hydrogel. Exogenous MBV can be derived from the same species, or a different species, than the ECM used to make an ECM hydrogel.

Gel: A state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (for example, the shape is discrete enough to maintain three dimensions on a two dimensional surface). “Gelation time,” also referred to as “gel time,” refers to the time it takes for a composition to become non-flowable under modest stress.

Gelation: The formation of a gel from a sol.

Hydrogel: A network of polymer chains that are hydrophilic, sometimes found as a colloidal gel in which water is the dispersion medium. Hydrogels are highly absorbent natural or synthetic polymeric networks. Hydrogels also possess a degree of flexibility similar to natural tissue. An “acoustic” hydrogel, such as an acoustic ECM hydrogel, is produced using ultrasound energy. The characteristics of these hydrogels are disclosed herein. For a hydrogel, the G′ (storage modulus) is typically about an order of magnitude greater than the G″ (loss modulus). An “enzymatic” ECM hydrogel, is produced by enzymatically digested ECM. The viscosity of an enzymatic hydrogel increases when warmed to physiological temperatures approaching about 37° C. For example, an enzymatic hydrogel is formed from an injectable solution at temperatures lower than 37° C. which forms a gel at a physiological temperature of 37° C.

Inflammation: Inflammation is a localized protective response elicited by injury to tissue that serves to sequester the inflammatory agent. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is characterized by an accumulation of white blood cells, either systemically or locally at the site of inflammation. The inflammatory response may be measured by many methods, including, but not limited to, measuring the number of white blood cells, the number of polymorphonuclear neutrophils (PMN), a measure of the degree of PMN activation, such as luminol enhanced-chemiluminescence, or a measure of the amount of cytokines present. C-reactive protein is a marker of a systemic inflammatory response.

An inflammatory disorder is a genus of disorders in which inflammation disrupts normal or regular physiological function. Inflammatory disorders can include a variety of conditions, such as autoimmune disorders (an inappropriate inflammatory response to an endogenous antigen), and disorders caused by inflammation due to traumatic injury or exogenous antigens. A primary inflammatory disorder is a disease or disorder that is caused by inflammation itself. A secondary inflammatory disorder is inflammation that is the result of another disorder. Inflammation can lead to inflammatory disorders, e.g., acute respiratory distress syndrome (ARDS).

In some aspects, anti-inflammatories are administered to treat an inflammatory disease or disorder, e.g., ARDS. Anti-inflammatories include, without limitation, nonsteroidal anti-inflammatory drugs (NSAIDs, for example, aspirin, ibuprofen, and naproxen), antileukotrienes, immune selective anti-inflammatory derivatives (ImSAIDs), bioactive compounds, steroids (such as corticosteroids), and opioids.

Isolated: An “isolated” biological component (such as a nucleic acid, protein cell, or nanovesicle) has been substantially separated or purified away from other biological components in the cell of the organism or the ECM, in which the component naturally occurs. Nucleic acids and proteins that have been “isolated” include nucleic acids and proteins purified by standard purification methods. MBV that have been isolated are removed from the fibrous materials of the ECM. The term also embraces nucleic acids and proteins prepared by recombinant expression in a host cell as well as chemically synthesized nucleic acids.

Isotonic Buffered Solution: A solution that is buffered to a pH between 7.2 and 7.8 and that has a balanced concentration of salts to promote an isotonic environment.

Lysyl oxidase (Lox): A copper-dependent enzyme that catalyzes formation of aldehydes from lysine residues in collagen and elastin precursors. These aldehydes are highly reactive, and undergo spontaneous chemical reactions with other lysyl oxidase-derived aldehyde residues, or with unmodified lysine residues. In vivo, this results in cross-linking of collagen and elastin, which plays a role in stabilization of collagen fibrils and for the integrity and elasticity of mature elastin. Complex cross-links are formed in collagen (pyridinolines derived from three lysine residues) and in elastin (desmosines derived from four lysine residues) that differ in structure. The genes encoding Lox enzymes have been cloned from a variety of organisms (Hamalainen et al., Genomics 11:508, 1991; Trackman et al., Biochemistry 29:4863, 1990; incorporated herein by reference). Residues 153-417 and residues 201-417 of the sequence of human lysyl oxidase have been shown to be important for catalytic function. There are four Lox-like isoforms, called LoxL1, LoxL2, LoxL3 and LoxL4.

Macrophage: A type of white blood cell that phagocytoses and degrades cellular debris, foreign substances, microbes, and cancer cells. In addition to their role in phagocytosis, these cells play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (CD86+/CD68+), whereas macrophages that decrease inflammation and encourage and regulate tissue repair are called M2 macrophages (CD206+/CD68+). The markers that identify the various phenotypes of macrophages vary among species. It should be noted that macrophage phenotype is represented by a spectrum that ranges between the extremes of M1 and M2. F4/80 (encoded by the adhesion G protein coupled receptor E1 (ADGRE1) gene) is a macrophage marker, see GENBANK® Accession No. NP_001243181.1, Apr. 6, 2018, and NP_001965, Mar. 5, 2018, both incorporated herein by reference. Without wishing to be bound by theory, it is believed that MBV have the ability to modulate the phenotype of macrophages, leading to an increase in M2-like, regulatory, or pro-remodeling macrophages. The effect of MBV on macrophages is further characterized in PCT Publication No. WO 2017/151862A1, incorporated herein by reference in its entirety. In some aspects, MBV of the present invention can be used to induce an M2 phenotype in macrophages and inhibit M1 macrophages in a subject.

MicroRNA: A small non-coding RNA that is about 17 to about 25 nucleotide bases in length, that post-transcriptionally regulates gene expression by typically repressing target mRNA translation. A microRNA (“miRNA” or “miR”) can function as negative regulators, such that greater amounts of a specific miRNA will correlates with lower levels of target gene expression. There are three forms of miRNAs, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nucleotide overhang at the 3′ end. The cleavage product, the premature miRNA (pre-miRNA) is about 60 to about 110 nucleotides long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nucleotides in length.

Nanovesicle: An extracellular vesicle that is a nanoparticle of about 10 to about 1,000 nm in diameter. Nanovesicles are lipid membrane bound particles that carry biologically active signaling molecules (e.g. microRNAs, proteins) among other molecules. Generally, the nanovesicle is limited by a lipid bilayer, and the biological molecules are enclosed and/or can be embedded in the bilayer. Thus, a nanovesicle includes a lumen surrounded by plasma membrane. The different types of vesicles can be distinguished based on diameter, subcellular origin, density, shape, sedimentation rate, lipid composition, protein markers, nucleic acid content and origin, such as from the extracellular matrix or secreted. A nanovesicle can be identified by its origin, such as a matrix bound nanovesicle from an ECM (see above), protein content and/or the miR content.

An “exosome” or “liquid phase extracellular vesicle (EV)” is a membranous vesicle which is secreted by a cell, and ranges in diameter from 10 to 150 nm. Generally, late endosomes or multivesicular bodies contain intralumenal vesicles which are formed by the inward budding and scission of vesicles from the limited endosomal membrane into these enclosed vesicles. These intralumenal vesicles are then released from the multivesicular body lumen into the extracellular environment, typically into a body fluid such as blood, cerebrospinal fluid or saliva, during exocytosis upon fusion with the plasma membrane. An exosome is created intracellularly when a segment of membrane invaginates and is endocytosed. The internalized segments which are broken into smaller vesicles and ultimately expelled from the cell contain proteins and RNA molecules such as mRNA and miRNA. Plasma-derived exosomes largely lack ribosomal RNA.

Extra-cellular matrix derived exosomes include specific miRNA and protein components, and have been shown to be present in virtually every body fluid such as blood, urine, saliva, semen, and cerebrospinal fluid. Exosomes can express CD11c, CD63, CD81, and/or CD9, and thus can be CD11c+ and/or CD63+ and/or C81+ and/or CD9+. Exosomes do not have high levels of lysyl oxidase on their surface.

A “nanovesicle derived from an ECM,” “matrix bound nanovesicle,” “MBV” or an “ECM-derived nanovesicle” all refer to the same membrane bound particles, ranging in size from 10 nm-1000 nm, present in the extracellular matrix, which contain biologically active signaling molecules such as protein, lipids, nucleic acid, growth factors and cytokines that influence cell behavior. The terms are interchangeable, and refer to the same vesicles. These nanovesicles are embedded within, and bound to, the ECM and are not simply attached to the surface or circulating freely in body fluids. These nanovesicles are resistant to harsh isolation conditions, such as freeze-thawing and digestion with proteases such as pepsin, elastase, hyaluronidase, proteinase K, and collagenase, and digestion with detergents. MBV are distinct from other extracellular vesicles including exosomes and have a phospholipid composition distinct from exosomes. MBV are distinct from bone matrix vesicles and do not express alkaline phosphatase. In certain circumstances, MBV can also be distinguished from exosomes based on the absence of certain markers commonly attributed to exosomes.

In some aspects, MBV are characterized by one or more of the following features of protein expression or lipid content:
(i) MBV may not express one or more of CD63 and/or CD81 and/or CD9 or have low or barely detectable levels of CD63 and/or CD81 and/or CD9 (CD63^{lo} and/or CD81^{lo} and/or CD910) (see, e.g., Example 1) compared with other vesicles, such as exosomes. A variety of methods can be used to distinguish low, barely detectable, or absent expression of CD63 and/or CD81 and/or CD9 in MBV, for example, antibody-based methods, such as western blotting or flow cytometry (see, e.g., Bashashati and Brinkman, Adv Bioinformatics, 2009:584603). In some aspects, MBV expression of CD63 and/or CD81 and/or CD9 is considered low or barely detectable compared with other vesicles where the expression of CD63 and/or CD81 and/or CD9 in MBV is at least one standard deviation or at least two standard deviations below the mean expression of other vesicles, such as exosomes;
(ii) MBV have a phospholipid content wherein at least 55% of total phospholipids comprise phosphatidylcholine (PC) and phosphatidyl inositol (PI) in combination;
(iii) MBV have a phospholipid content wherein 10% or less of total phospholipids comprise sphingomyelin (SM);
(iv) MBV have a phospholipid content wherein 20% or less of total phospholipids comprise phosphatidylethanolamine (PE);
(v) MBV have a phospholipid content wherein 15% or greater of the total phospholipid content comprises phosphatidylinositol (PI) with the percent representing the percent of lipid concentration.

In some aspects, MBV are characterized by all of the following features:
(i) do not express one or more of CD63 and/or CD81 and/or CD9 or have low or barely detectable levels of CD63 and/or CD81 and/or CD9 (CD63^{lo} and/or CD811 and/or CD910) (as further described above);
(ii) a phospholipid content wherein at least 55% of total phospholipids comprise phosphatidylcholinc (PC) and phosphatidyl inositol (PI) in combination;
(iii) a phospholipid content wherein 10% or less of total phospholipids comprise sphingomyelin (SM);
(iv) a phospholipid content wherein 20% or less of total phospholipids comprise phosphatidylethanolamine (PE); and
(v) a phospholipid content wherein 15% or greater of the total phospholipid content is phosphatidylinositol (PI).

In some aspects, MBV are characterized by all of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some aspects, MBV are characterized by one or more of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some aspects, MBV are characterize by one or more of the following features:
(i) do not contain detectable levels of alkaline phosphatase;
(ii) do not contain detectable levels of osteopontin;
(iii) do not contain detectable levels of osteoprogeterin;
(iv) do not contain detectable levels of complement C5; and/or
(v) do not contain detectable levels of c-reactive protein.

In some aspects, MBV contain IL33 and are IL33⁺.

The ECM from which MBV are isolated can be an ECM from a tissue, can be produced from cells in culture, or can be purchased from a commercial source.

Pharmaceutically acceptable carriers: The pharmaceutically acceptable carriers useful in the claimed pharmaceutical preparations are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical preparations to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell.

Phospholipid: A class of lipids having a structure consisting of two hydrophobic fatty acid tails and a hydrophilic head consisting of a phosphate group. Major classes of phospholipids include phosphatidylcholine (PC), phosphatidylethanolamine (PE), phosphatidylinositol (PI), phosphatidylserine (PS), phosphatidylglycerol (PG), sphingomyelin (SM), cardiolipin (CL), phosphatidic acid (PA), and bis-monoacylglycerophosphate (BMP). Phospholipids can be measured in a variety of ways. For example, liquid chromatography-mass spectrometry (LC-MS) based global lipidomics and redox lipidomics can be used. In some aspects, specific phospholipid content is indicated as the percent concentration of the total phospholipids (such as total phospholipids in MBV), where the percent concentration is weight/weight (w/w).

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Prophylactic: as used herein refers to a medication or a treatment designed and used to prevent a disease or disorder from occurring. As used herein, the terms “prophylactic” and “prevention” are used interchangeably.

Purified: The term “purified” does not require absolute purity; rather, it is intended as a relative term. Thus, for example, a purified nucleic acid molecule preparation is one in which the nucleic referred to is more pure than the nucleic in its natural environment within a cell. For example, a preparation of a nucleic acid is purified such that the nucleic acid represents at least 50% of the total protein content of the preparation. Similarly, a purified MBV preparation is one in which the exosome is more pure than in an environment including cells, wherein there are microvesicles and exosomes. A purified population of nucleic acids or MBV is greater than about 90%, 91%, 92%, 93%, 94%, 95%, 96%, 97%, 98%, 99% or 100% pure, or free other nucleic acids or cellular components, respectively.

Preventing or treating a disease: “Preventing” a disease refers to inhibiting the development of a disease, for example in a person who is known to have a predisposition to a disease. An example of a person with a known predisposition is someone with a history of a disease in the family, or who has been exposed to factors that predispose the subject to a condition. “Treatment” refers to a therapeutic intervention that ameliorates a sign or symptom of a disease or pathological condition after it has begun to develop.

Solubilized ECM: ECM that has been treated with ultrasonic cavitation or enzymatic digestion thereby causing micro-structural changes, such as by physical disruption of protein aggregates or digestion, respectively.

Subject: Human and non-human animals, including all vertebrates, such as mammals and non-mammals, such as non-human primates, mice, rabbits, sheep, dogs, cats, horses, cows, chickens, amphibians, and reptiles. In some aspects of the described methods, the subject is a human. “Subject” is used interchangeably with the term “patient.” A subject may be an individual diagnosed with a high risk of developing a disease or disorder, for example, an infectious disease or disorder (e.g., an immunocompromised individual, a healthcare professional), someone who has been diagnosed with a disease or disorder, for example, an infectious disease or disorder, someone who previously suffered from a disease or disorder, for example, an infectious disease or disorder, or an individual evaluated for symptoms or indications of a disease or disorder, for example, an infectious disease or disorder.

Therapeutically effective amount: A quantity of a specific substance, such as an MBV, sufficient to achieve a desired effect in a subject being treated. When administered to a subject, a dosage will generally be used that will achieve target tissue concentrations (for example, in the lung) that has been shown to achieve a desired in vitro effect.

Thermoreversible hydrogel: Hydrogel formed due to entanglement of polymer chains wherein the viscosity changes at a characteristic temperature of gelation. The disclosed acoustic ECM hydrogels are thermoreversible hydrogels that show gelation (sol to gel transition) upon cooling.

Topical application: A topically applied agent is applied only in a specific area, and not throughout the body. In particular examples the composition is applied to the skin or the eye in an area where hemostasis is desired. For example the pharmaceutical composition can be applied in a topical preparation to a wound, such as an epithelial wound or defect, for example a traumatic or surgical wound, such as a skin or corneal abrasion or surgical incision.

Total phospholipid content: “Total phospholipids” or “total phospholipid content”, as used herein, with respect to MBV, refers to the sum of all phospholipids present in a given quantity of isolated MBV, i.e., MBV isolated from the ECM. MBV can be isolated, for example, by enzymatic digestion of decellularized ECM and differential centrifugation. The total phospholipid content can be determined by methods such as LC-MS based global lipidomics and redox lipidomics. The total phospholipid content is measured by weight. A percentage of the total phospholipid content refers to a percent concentration on a weight/weight basis

Transplanting: The placement of a biocompatible substrate, such as an MBV, into a subject in need thereof.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, or improving a subject's physical or mental well-being. The treatment may be assessed by objective or subjective parameters; including the results of a physical examination, neurological examination, or psychiatric evaluations.

Ultrasonication: The process of exposing ultrasonic waves with a frequency higher than 20 kHz.

### Overview

In some aspects, a composition is disclosed that includes an extracellular matrix (ECM) hydrogel including a) solubilized extracellular matrix; and b) exogenous matrix bound nanovesicles (MBV) derived from extracellular matrix, wherein the MBV do not express CD63 and CD81 or are CD6310CD811 and wherein the MBV do not contain alkaline phosphatase. The exogenous MBV are present in the ECM hydrogel at a concentration of less than 1 mg/mL. For example, the MBV may be present in the ECM hydrogel at a concentration of at least about 1×10⁵ to about 1×10²⁰ particles/mL. In another example, the MBV may be present in the ECM hydrogel at a concentration of about 1×10⁶ to about 1×10¹² particles/mL.

In some aspects, a composition is disclosed that includes an extracellular matrix (ECM) hydrogel including a) solubilized extracellular matrix; and b) exogenous matrix bound nanovesicles (MBV) derived from extracellular matrix, wherein the MBV do not express CD63 and CD81 or are CD63^{lo}CD81^{lo} and wherein the MBV do not contain alkaline phosphatase. The exogenous MBV are present in the ECM hydrogel at a concentration of less than 1 mg/mL. For example, the MBV may be present in the ECM hydrogel at a concentration of at least about 1×10⁵ to about 1×10²⁰ particles/mL. In some aspects, the composition has one or more of the following features: i) is sheer thinning; ii) has a storage modulus (G′) of about 50 Pa to about 200 Pa, and a loss modulus (G″) of about 5 Pa to about 20 Pa, and a G′ to G″ ratio of about 4:1 to about 15:1 at 37° C., and/or iii) has a 50% degradation rate of 24 hours to 14 days.

In some aspects, a composition is disclosed that includes an extracellular matrix (ECM) hydrogel including a) solubilized extracellular matrix; and b) exogenous matrix bound nanovesicles (MBV) derived from extracellular matrix, wherein the MBV do not express CD63 and CD81 or are CD6310CD81^{lo} and wherein the MBV do not contain alkaline phosphatase. The exogenous MBV are present in the ECM hydrogel at a concentration of less than 1 mg/mL. For example, the MBV may be present in the ECM hydrogel at a concentration of at least about 1×10⁵ to about 1×10²⁰ particles/mL. In some aspects, the composition i) is sheer thinning; ii) has a storage modulus (G′) of about 50 Pa to about 200 Pa, and a loss modulus (G″) of about 5 Pa to about 20 Pa, and a G′ to G″ ratio of about 4:1 to about 15:1 at 37° C., and iii) has a 50% degradation rate of 24 hours to 14 days. In some aspects, the amount of solubilized ECM in the ECM hydrogel is between 1 mg/mL and 500 mg/mL. In some aspects, the amount of solubilized ECM in the ECM hydrogel is 1 mg/mL to 400 mg/mL, or 1 mg/mL to 350 mg/mL, or 1 mg/mL to 300 mg/mL, or 1 mg/mL to 250 mg/mL, or 1 mg/mL to 200 mg/mL, or 1 mg/mL to 150 mg/mL, or 1 mg/mL to 100 mg/mL, or 1 mg/mL to 50 mg/mL, or 5 mg/mL to 250 mg/mL, or 20 mg/mL to 200 mg/mL, or 5 mg/mL to 200 mg/mL, or 5 mg/mL to 100 mg/mL. In more aspects, the amount of solubilized ECM in the ECM hydrogel is between about 5 mg/ml to about 50 mg/ml, such as about 10 mg/ml to about 50 mg/ml, about 20 mg/ml to about 50 mg/ml, about 30 mg/ml to about 50 mg/ml, about 40 mg/ml to about 50 mg/ml, about 5 mg/ml to about 40 mg/ml, about 5 mg/ml to about 30 mg/ml, about 5 mg/ml to about 20 mg/mg, or about 5 mg/ml to about 10 mg/ml. The ECM hydrogel can contain solubilized ECM at a concentration of about 5, 10, 15, 20, 25, 30, 35, 40, 45, 50, 60, 65, 70, 75, 80, 85, 90, 95, 100, 150, 200, 250, 300, 350, 400, 450, or 500 mg/ml. In one non-limiting example, the amount of solubilized ECM in the ECM hydrogel is between 10 mg/mL and 30 mg/mL. In one non-limiting example, the amount of solubilized ECM in the ECM hydrogel is between 4 mg/mL and 50 mg/mL. In some aspects, the ECM in the ECM hydrogel or solubilized ECM is present at a concentration of about 5 to about 100 mg/mL, e.g., 50 to 100 mg/mL, 25 to 75 mg/mL, 60 to 80 mg/mL, 40 to 60 mg/mL, 50 to 80 mg/mL, or about 30 to about 60 mg/mL.

In some aspects, the exogenous MBV are present in the ECM hydrogel at a concentration of about 1×10⁵ to about 1×10¹⁸ particles/mL, such as about 1×10⁵ to about 1×10¹⁶ particles/mL, such as about 1×10⁵ to about 1×10¹⁴ particles/mL, or such as about 1×10⁵ to about 1×10¹² particles/mL. In some aspects, the exogenous MBV are present in the ECM hydrogel at a concentration of about 1×10⁶ to about 1×10²⁰ particles/mL, such as about 1×10⁶ to about 1×10¹⁸ particles/mL, such as about 1×10⁶ to about 1×10¹⁶ particles/mL, such as about 1×10⁶ to about 1×10¹⁴ particles/mL, such as about 1×10⁶ to about 1×10¹² particles/mL, such as about 1×10⁷ to about 1×10¹² particles/mL, such as about 1×10⁷ to about 1×10¹¹ particles/mL, such as about 1×10⁸ to about 1×10¹² particles/mL, such as about 1×10⁸ to about 1×10¹¹ particles/mL, such as about 1×10⁹ to about 1×10¹² particles/mL, such as about 1×10⁹ to about 1×10¹¹ particles/mL, such as about 1×10¹⁰ to about 1×10¹² particles/mL, such as about 1×10¹⁰ to about 1×10¹¹ particles/mL, such as about 1×10¹¹ to about 1×10¹² particles/mL. In one non-limiting example, the exogenous MBV are present in the ECM hydrogel at a concentration of about 1×10⁸ to 1×10¹¹ particles/mL. In one non-limiting example, the exogenous MBV are present in the ECM hydrogel at a concentration of about 1×10⁵ to 1×10¹² particles/mL. In one non-limiting example, the exogenous MBV are present in the ECM hydrogel at a concentration of about 1×10⁶ to 1×10¹² particles/mL. In some aspects, the exogenous MBV are present in the ECM hydrogel at a concentration of about 1×10⁶, 1×10⁷, 1×10⁸, 1×10⁹, 1×10¹⁰, 1×10¹¹, 1×10¹², 1×10¹⁴, 1×10¹⁶, 1×10¹⁸, or about 1×10²⁰ particles/mL. In other aspects, the exogenous MBV are present in the ECM hydrogel at a concentration of about 5×10⁶, 5×10⁷, 5×10⁸, 5×10⁹, 5×10¹⁰, 5×10¹¹, or about 5×10¹² particles/mL. In a specific non-limiting example, the exogenous MBV are present in the ECM hydrogel at a concentration of about 1×10¹¹ particles/mL. In another non-limiting example, the exogenous MBV are present in the ECM hydrogel at a concentration of about 1×10¹² particles/mL. In a further non-limiting example, the exogenous MBV are present in the ECM hydrogel at concentration of about 1×10¹⁰ particles/mL or about 1×10⁹ particles/mL. In some aspects, the exogenous MBV are present in the ECM hydrogel at a concentration of about 1×10⁶ to 1×10¹⁸ particles/mL, e.g. about 1×10⁶ to 1×10¹⁴, about 1×10¹⁰ to 1×10¹⁴, 1×10¹² to 1×10¹⁸, 1×10¹⁴ to 1×10¹⁸, or 1×10¹⁰ to 1×10¹⁸. In another non-limiting example, the exogenous MBV are present in the ECM hydrogel at a concentration of less than 1 mg/mL, e.g., ≤0.9 mg/mL, ≤0.8 mg/mL, ≤0.7 mg/mL, ≤0.6 mg/mL, ≤0.5 mg/mL, ≤0.4 mg/mL, ≤0.3 mg/mL, ≤0.2 mg/mL, ≤0.1 mg/mL, ≤90 μg/mL, ≤80 μg/mL, ≤70 μg/mL, ≤60 μg/mL, ≤50 μg/mL, ≤40 μg/mL, ≤30 μg/mL, ≤20 μg/mL, or ≤10 μg/mL, but greater than 0 μg/mL, e.g., greater than 0.1 μg/mL, or greater than 0.5 μg/mL, or greater than 1 μg/mL.

In some aspects, the composition has a storage modulus (G′) of about 50 Pa to about 200 Pa, such as about 75 Pa to about 200 Pa, about 100 Pa to about 200 Pa, about 125 Pa to about 200 Pa, about 150 Pa to about 200 Pa, about 175 Pa to about 200 Pa, about 50 Pa to about 75 Pa, about 50 to about 100 Pa, about 50 to about 125 Pa, about 50 to about 150 Pa, or about 50 to about 175 Pa. The composition can have a storable modulus of about 50, 60, 70, 80. 90, 100, 110, 120, 130, 140, 150, 160, 170, 180, 190 or 200 Pa. The composition can have a storable modulus of about 50-60, 60-70, 70-80, 80-90, 90-100, 100-110, 110-120, 120-130, 130-140, 150-150, 150-160, 160-170, 170-180, 180-190 or 190-200 Pa.

In more aspects, the composition has a loss modulus (G″) of about 5 Pa to about 20 Pa, such as about 10 Pa to about 20 Pa, about 15 to about 20 Pa, about 5 Pa to about 10 Pa, about 5 Pa to about 15 Pa. The composition can have a loss modulus of about 5, 6, 7, 8, 9, 10, 11, 12, 13, 14, 15, 16, 17, 18, 19 or 20 Pa.

In further aspects the composition has a G′ to G″ ratio of about 4:1 to about 15:1 at 37° C. In some aspects, the composition can have a G′ to G″ ratio of about 4:1 to about 5:1, about 4:1 to about 6:1, about 4:1 to about 7:1, about 4:1 to about 8:1, about 4:1 to about 9:1, about 4:1 to about 10:1, about 4:1 to about 11:1, about 4:1 to about 12:1, about 4:1 to about 13:1, or about 4:1 to about 14:1. In other aspects, the composition can have a G′ to G″ ratio of about 5:1 to about 15:1, about 6:1 to about 15:1, about 7:1 to about 15:1, about 8:1 to about 15:1, about 9:1 to about 15:1, about 10:1 to about 15:1, about 11:1 to about 15:1, about 12:1 to about 15:1, about 13:1 to about 15:1, or about 14:1 to about 15:1. The composition can have a G to G″ ratio of about 4:1, 5:1, 6:1, 7:1, 8:1, 9:1, 10:1, 11:1, 12:1, 13:1, 14:1 or 15:1.

In other aspects, the composition is sheer thinning so that the ease of delivery via injection becomes easier as the rate of injection (e.g., ml/sec) increases. In some non-limiting examples, as the sheer rate (expressed as 1/sec) increases, the viscosity (expressed as Pa* see) decreases.

In more aspects, the composition has a 50% degradation rate of 24 hours to 14 days. The composition can have a 50% degradation rate of about 1 to about 13 days, about 1 to about 12 days, about 1 to about 11 days, about 1 to about 10 days, about 1 to about 9 days, about 1 to about 8 days, about 1 to about 7 days, about 1 to about 6 days, about 1 to about 5 days, about 1 to about 4 days, about 1 to about 3 days about 1 to about 2 days. The composition can have a 50% degradation rate of about 2 to about 14 days, about 3 to about 14 days, about 4 to about 14 days, about 5 to about 14 days, about 6 to about 14 days, about 7 to about 14 days, about 8 to about 14 days, about 9 to about 14 days, about 10 to about 14 days, about 11 to about 14 days, about 12 to about 14 days, or about 13 to about 14 days. The composition can have a 50% degradation rate of about 1, 2, 3, 4, 5, 6, 7, 8, 9, 01, 11, 12, 13, or 14 days. The rate of release of MBV from the hydrogels following delivery to an anatomic site can depend upon the degradation profile of the hydrogel, the anatomic site, and the degree of inflammation of the tissue.

In some aspects, the ECM hydrogel can be an acoustic ECM hydrogel. Acoustic hydrogels suitable for use according to the instant application are disclosed, for example, in PCT Publication No. WO2020/186082, incorporated herein by reference. In other aspects, the ECM hydrogel can be an enzymatic ECM hydrogel. Enzymatic ECM hydrogels suitable for use according to the instant application are disclosed, for example, in U.S. Pat. No. 8,361,503, which is incorporated by reference herein in its entirety.

In other aspects, compositions are disclosed that include an acidic solution comprising an exogenous acid protease and solubilized extracellular matrix, e.g., solubilized intact ECM; and exogenous MBV derived from extracellular matrix, wherein the MBV do not express CD63 and CD81 or are CD631CD8110 and wherein the MBV do not contain alkaline phosphatase. The exogenous MBV are present in the composition at a concentration in an amount less than 1 mg/mL. For example, the MBV are present in the composition in the amount of about 1×10⁵ to about 1×10²⁰ particles/ml, and the acidic solution, when neutralized to a pH of between about 7.0 to about 7.8 forms a gel at a temperature greater than 25° C. In some aspects, the acid protease is pepsin and/or trypsin. In some aspects, the pH of the composition is less than 7.0.

In more aspects, compositions are disclosed that include solubilized extracellular matrix, e.g., intact ECM, that has been digested by an acid protease; a deactivated exogenous acid protease; and exogenous MBV derived from extracellular matrix. The MBV do not express CD63 and CD81 or are CD631CD8110 and do not contain alkaline phosphatase. These exogenous MBV are present in the composition at a concentration in an amount less than 1 mg/mL. For example, the MBV are present in the composition in the amount of about 1×10⁵ to about 1×10²⁰ particles/ml. The composition enters the liquid phase at a temperature less than 25° C. and enters a gel phase at a temperature greater than 25° C., and has a pH of between 7 and 7.8, such as about 7.2 to about 7.8, about 7.3 to 7.8, 7.4 to 7.8 or about 7.5 to 7.8. The composition can have a pH for example, about 7.2, about 7.3, about 7.4, about 7.5 or about 7.6. In some aspects, the acid protease is pepsin and/or trypsin. In other aspects, the composition has a pH of about 7.2.

In more aspects, the composition has a pH in the range of 7.2 to 7.4, such as about 7.2, 7.3 or 7.4.

In some aspects, the amount of solubilized ECM in the disclosed compositions is between 1 mg/mL and 500 mg/mL. In some aspects, the amount of solubilized ECM in the disclosed compositions is 1 mg/mL to 400 mg/mL, or 1 mg/mL to 350 mg/mL, or 1 mg/mL to 300 mg/mL, or 1 mg/mL to 250 mg/mL, or 1 mg/mL to 200 mg/mL, or 1 mg/mL to 150 mg/mL, or 1 mg/mL to 100 mg/mL, or 1 mg/mL to 50 mg/mL, or 5 mg/mL to 250 mg/mL, or 20 mg/mL to 200 mg/mL, or 5 mg/mL to 200 mg/mL, or 5 mg/mL to 100 mg/mL. In more aspects, the amount of solubilized ECM in the disclosed compositions is between about 5 mg/ml to about 50 mg/ml, such as about 10 mg/ml to about 50 mg/ml, about 20 mg/ml to about 50 mg/ml, about 30 mg/ml to about 50 mg/ml, about 40 mg/ml to about 50 mg/ml, about 5 mg/ml to about 40 mg/ml, about 5 mg/ml to about 30 mg/ml, about 5 mg/ml to about 20 mg/mg, or about 5 mg/ml to about 10 mg/ml. The disclosed compositions can contain solubilized ECM at a concentration of about 5, 10, 15, 20, 25, 30, 35, 40, 45, 50, 60, 65, 70, 75, 80, 85, 90, 95, 100, 150, 200, 250, 300, 350, 400, 450, or 500 mg/ml. In one non-limiting example, the amount of solubilized ECM in the disclosed compositions is between 10 mg/mL and 30 mg/mL. In one non-limiting example, the amount of solubilized ECM in the disclosed compositions is between 4 mg/mL and 50 mg/mL. In some aspects, the ECM in the disclosed compositions or solubilized ECM is present at a concentration of about 5 to about 100 mg/mL, e.g., 50 to 100 mg/mL, 25 to 75 mg/mL, 60 to 80 mg/mL, 40 to 60 mg/mL, 50 to 80 mg/mL, or about 30 to about 60 mg/mL.

In some aspects, the exogenous MBV are present in the disclosed compositions at a concentration of about 1×10⁵ to about 1×10²⁰ particles/mL, such as about 1×10⁵ to about 1×10¹⁸ particles/mL such as about 1×10⁵ to about 1×10¹⁶ particles/mL, such as about 1×10⁵ to about 1×10¹⁴ particles/mL, or such as about 1×10⁵ to about 1×10¹² particles/mL. In some aspects, the exogenous MBV are present in the disclosed compositions at a concentration of about 1×10⁶ to about 1×10²⁰ particles/mL, such as about 1×10⁶ to about 1×10¹⁸ particles/mL, such as about 1×10⁶ to about 1×10¹⁶ particles/mL, such as about 1×10⁶ to about 1×10¹⁴ particles/mL, such as about 1×10⁶ to about 1×10¹² particles/mL, such as about 1×10⁷ to about 1×10¹² particles/mL, such as about 1×10⁷ to about 1×10¹¹ particles/mL, such as about 1×10⁸ to about 1×10¹² particles/mL, such as about 1×10⁸ to about 1×10¹¹ particles/mL, such as about 1×10⁹ to about 1×10¹² particles/mL, such as about 1×10⁹ to about 1×10¹¹ particles/mL, such as about 1×10¹⁰ to about 1×10¹² particles/mL, such as about 1×10¹⁰ to about 1×10¹¹ particles/mL, such as about 1×10¹¹ to about 1×10¹² particles/mL. In one non-limiting example, the exogenous MBV are present in the disclosed compositions at a concentration of about 1×10⁸ to 1×10¹¹ particles/mL. In one non-limiting example, the exogenous MBV are present in the disclosed compositions at a concentration of about 1×10⁵ to 1×10¹² particles/mL. In one non-limiting example, the exogenous MBV are present in the disclosed compositions at a concentration of about 1×10⁶ to 1×10¹² particles/mL. In some aspects, the exogenous MBV are present in the disclosed compositions at a concentration of about 1×10⁶, 1×10⁷, 1×10⁸, 1×10⁹, 1×10¹⁰, 1×10¹¹, 1×10¹², 1×10¹⁴, 1×10¹⁶, 1×10¹⁸, or about 1×10²⁰ particles/mL. In other aspects, the exogenous MBV are present in the disclosed compositions at a concentration of about 5×10⁶, 5×10⁷, 5×10⁸, 5×10⁹, 5×10¹⁰, 5×10¹¹, or about 5×10¹² particles/mL. In a specific non-limiting example, the exogenous MBV are present in the disclosed compositions at a concentration of about 1×10¹¹ particles/mL. In another non-limiting example, the exogenous MBV are present in the disclosed compositions at a concentration of about 1×10¹² particles/mL. In a further non-limiting example, the exogenous MBV are present in the disclosed compositions at concentration of about 1×10¹⁰ particles/mL or about 1×10⁹ particles/mL. In some aspects, the exogenous MBV are present in the disclosed compositions at a concentration of about 1×10⁶ to 1×10¹⁸ particles/mL, e.g. about 1×10⁶ to 1×10¹⁴, about 1×10¹⁰ to 1×10¹⁴, 1×10¹² to 1×10¹⁸, 1×10¹⁴ to 1×10¹⁸, or 1×10¹⁰ to 1×10¹⁸. In another non-limiting example, the exogenous MBV are present in the ECM hydrogel at a concentration of less than 1 mg/mL, e.g., ≤0.9 mg/mL, ≤0.8 mg/mL, ≤0.7 mg/mL, ≤0.6 mg/mL, ≤0.5 mg/mL, ≤0.4 mg/mL, ≤0.3 mg/mL, ≤0.2 mg/mL, ≤0.1 mg/mL, ≤90 μg/mL, ≤80 μg/mL, ≤70 μg/mL, ≤60 μg/mL, ≤50 μg/mL, ≤40 μg/mL, ≤30 μg/mL, ≤20 μg/mL, or ≤10 μg/mL, but greater than 0 μg/mL, e.g., greater than 0.1 μg/mL, or greater than 0.5 μg/mL, or greater than 1 μg/mL.

In some aspects, the ECM hydrogel, or the solubilized ECM of the disclosed compositions is derived from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus. In more aspects, the ECM hydrogel, or the solubilized ECM of the disclosed compositions is derived from urinary bladder matrix (UBM), small intestinal submucosa (SIS), or urinary bladder submucosa (UBS). In further aspects, the ECM hydrogel, or the solubilized ECM of the disclosed compositions is derived from a mammalian vertebrate selected from a human, monkey, pig, cow, or sheep. In yet other aspects, the ECM hydrogel, or the solubilized ECM of the disclosed compositions is derived from a non-human mammal. In some aspects, the ECM hydrogel, or the solubilized ECM of the disclosed compositions is not derived from UBM.

In some aspects, the MBV do not express CD63 and CD81 or are CD631CD81^{lo} and the MBV do not contain detectable alkaline phosphatase. In some aspects, expression of CD63, CD81, and/or CD9 cannot be detected on the MBV. Thus, in some aspects the MBV do not express CD63 and/or CD81 and/or CD9. In one specific example, CD63, CD81, and CD9 cannot be detected on the nanovesicles. In other aspects, the MBV have barely detectable levels of CD63, CD81, and CD9, such as that detectable by Western blot. These MBV are CD63^{lo}CD81^{lo}CD9^{lo}. In other aspects, MBV do not express detectable levels of one or more of CD63, CD81, or CD9. In further aspects, the MBV do not contain detectable alkaline phosphatase, osteopontin, osteoprogeterin, complement C5, and/or c-reactive protein.

In more aspects, the MBV, e.g., the exogenous MBV, are derived from extracellular matrix of urinary bladder, small intestine, heart, dermis, liver, kidney, uterus, brain, blood vessel, lung, bone, muscle, pancreas, placenta, stomach, spleen, colon, adipose tissue, and/or esophagus. In more aspects, the MBV, e.g., the exogenous MBV, are derived from extracellular matrix of urinary bladder, small intestine, dermis, liver, kidney, uterus, brain, blood vessel, lung, muscle, pancreas, placenta, stomach, spleen, colon, adipose tissue, and/or esophagus. In specific non-limiting examples, the MBV, e.g., the exogenous MBV, are derived from urinary bladder matrix (UBM), small intestinal submucosa (SIS), or urinary bladder submucosa (UBS). In one aspect, the MBV, e.g., the exogenous MBV, are derived from dermis. In another aspect, the MBV, e.g., the exogenous MBV, are derived from UBM. In further aspects, the MBV, e.g., the exogenous MBV, are derived from extracellular matrix from a mammalian vertebrate selected from a human, monkey, pig, cow, or sheep. In specific non-limiting examples, the MBV, e.g., the exogenous MBV, are from a non-human mammal. In some aspects, the MBV, e.g., the exogenous MBV, are not derived from bone ECM. In some aspects, the MBV, e.g., the exogenous MBV, are not derived from heart (cardiac) ECM. In some aspects, the MBV, e.g., the exogenous MBV, are not derived from heart (cardiac) ECM or bone ECM.

In some aspects, the composition can include comminuted ECM. In other aspects, the composition can include trehalose.

The disclosed compositions can be formulated for topical administration. These topical composition are of use to treat inflammation. For example, the disclosed compositions can be used to treat inflammation in the esophagus, e.g., esophagitis or ulcers in the esophagus. In one aspect, the compositions of the invention are applied topically to coat the esophageal tissue. The disclosed compositions can be used to treat anal fistulas. The disclosed compositions can be used as a submucosal cushion.

Methods are disclosed herein for treating a subject with inflammatory bowel disease, or esophageal inflammation. These methods include administering topically to an affected organ of the subject an effective amount of a disclosed composition, thereby treating the inflammatory bowel disease or the esophageal inflammation in the subject. In some aspects, the subject has the inflammatory bowel disease, and the affected organ is the bowel. In other aspects, the subject has ulcerative colitis and the affected organ is the colon. In more aspects, the subject has esophageal inflammation and the affected organ is the esophagus. In some non-limiting examples, the subject is human.

The compositions may also be used to treat inflammation or to promote wound healing in the throat or stomach by topically application of the compositions disclosed herein to the throat or stomach of a subject suffering from inflammation or wound in the throat or stomach, e.g. a stomach ulcer or throat ulcer. The compositions may be applied topically through enteral administration, e.g., by mouth, or by application through a surgical procedure such as with a catheter or endoscope, or by injection to achieve local, administration at the site of the inflammation, ulcer, or wound.

Matrix Bound Nanovesicles, Derived from an Extracellular Matrix (ECM)

Nanovesicles derived from ECM (also called matrix bound nanovesicles, “MBV”) are generally described in PCT Publication Nos. WO 2017/151862, WO 2018/204848, and WO 2019/213482, incorporated herein by reference. It is disclosed that MBV are embedded in the extracellular matrix. These MBV can be isolated and are biologically active. MBV do not express CD63 and CD81 or are CD63^{lo}CD81^{lo} and do not contain alkaline phosphatase. The MBV can contain IL-33. These MBV can be used for therapeutic purposes. In some aspects, the MBV do not contain MBV do not contain alkaline phosphatase, osteopontin, osteoprogeterin, complement C5, and/or c-reactive protein.

An extracellular matrix is a complex mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. Generally, the disclosed MBV are embedded in any type of extracellular matrix (ECM), and can be isolated from this location. Thus, MBV are not detachably present on the surface of the ECM, and are not exosomes (also known as extracellular vesicles or EV).

Extracellular matrices are disclosed, for example and without limitation, in U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666; each of which is incorporated by reference in its entirety). However, an ECM can be produced from any tissue, or from any in vitro source wherein the ECM is produced by cultured cells and comprises one or more polymeric components (constituents) of native ECM. ECM preparations can be considered to be “decellularized” or “acellular”, meaning the cells have been removed from the source tissue or culture.

In some aspects, the ECM is isolated from a vertebrate animal, for example, from a mammalian vertebrate animal including, but not limited to, human, monkey, pig, cow, sheep, etc. The ECM may be derived from any organ or tissue, including without limitation, urinary bladder, intestine (such as small intestine or large intestine), heart, dermis, liver, kidney, uterus, brain, blood vessel, lung, bone, muscle, pancreas, placenta, stomach, spleen, colon, adipose tissue, or esophagus. In specific non-limiting examples, the extracellular matrix is isolated from esophageal tissue, urinary bladder (such as urinary bladder matrix or urinary bladder submucosa), small intestinal submucosa, dermis, umbilical cord, pericardium, cardiac tissue, or skeletal muscle. The ECM can comprise any portion or tissue obtained from an organ, including, for example and without limitation, submucosa, epithelial basement membrane, tunica propria, etc. In one non-limiting aspect, the ECM is isolated from urinary bladder. In some aspects, the ECM is from a human subject. In other aspects, the ECM is from a porcine subject. In some aspect, the ECM is not porcine ECM. In some aspects, the ECM is not porcine UBM.

The ECM may or may not include the basement membrane. In another non-limiting aspect, the ECM includes at least a portion of the basement membrane. The ECM material may or may not retain some of the cellular elements that comprised the original tissue such as capillary endothelial cells or fibrocytes. In some aspects, the ECM contains both a basement membrane surface and a non-basement membrane surface.

In some aspects, the ECM is harvested from porcine urinary bladders (also known as urinary bladder matrix or UBM). Briefly, the ECM is prepared by removing the urinary bladder tissue from a mammal, such as a pig, and trimming residual external connective tissues, including adipose tissue. All residual urine is removed by repeated washes with tap water. The tissue is delaminated by first soaking the tissue in a de-epithelializing solution, for example and without limitation, hypertonic saline (e.g., 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. The relatively fragile epithelial basement membrane is invariably damaged and removed by any mechanical abrasion on the luminal surface. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated. After these tissues are removed, the resulting ECM consists mainly of epithelial basement membrane and subjacent tunica propria.

In another aspect, the ECM is prepared by abrading porcine bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. Care is taken to prevent perforation of the submucosa. After these tissues are removed, the resulting ECM consists mainly of the tunica submucosa (see FIG. 2 of U.S. Pat. No. 9,277,999, which is incorporated herein by reference).

ECM can also be prepared as a powder. Such powder can be made according to the method of Gilbert et al., Biomaterials 26 (2005) 1431-1435, herein incorporated by reference in its entirety. For example, UBM sheets can be lyophilized and then chopped into small sheets for immersion in liquid nitrogen. The snap frozen material can then be comminuted so that particles are small enough to be placed in a rotary knife mill, where the ECM is powdered. Similarly, by precipitating NaCl within the ECM tissue the material will fracture into uniformly sized particles, which can be snap frozen, lyophilized, and powdered.

In one non-limiting aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another non-limiting aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to UBM (ACell Corporation; Jessup, Md.).

MBV can be derived from (released from) an extracellular matrix using the methods disclosed below. For example, MBV may be obtained from extracellular matrix according to the methods disclosed in U.S. Patent Application Publication No. 2019/0117837, the contents of which are incorporated by reference herein for all purposes. In some aspects, the ECM is digested with an enzyme, such as pepsin, collagenase, elastase, hyaluronidase, and/or proteinase K, and the MBV are isolated. In other aspects, the MBV are released and separated from the ECM by changing the pH with solutions such as glycine HCL, citric acid, ammonium hydroxide, use of chelating agents such as, but not limited to, EDTA, EGTA, by ionic strength and or chaotropic effects with the use of salts such as, but not limited to potassium chloride (KCl), sodium chloride, magnesium chloride, sodium iodide, sodium thiocyanate, or by exposing ECM to denaturing conditions like guanidine HCl or Urea.

The MBV may be derived from extracellular matrix of urinary bladder, small intestine, heart, dermis, liver, kidney, uterus, brain, blood vessel, lung, bone, muscle, pancreas, placenta, stomach, spleen, colon, adipose tissue, and/or esophagus. In specific non-limiting examples, the MBV are derived from urinary bladder matrix (UBM), small intestinal submucosa (SIS), or urinary bladder submucosa (UBS). In one aspect, the MBV are derived from dermis. In another aspect, the MBV are derived from UBM. In further aspects, the MBV are derived from extracellular matrix from a mammalian vertebrate selected from a human, monkey, pig, cow, or sheep. In specific non-limiting examples, the MBV are from a non-human mammal. In some aspects, the MBV are not derived from bone ECM. In some aspects, the MBV are not derived from heart (cardiac) ECM. In some aspects, the MBV are not derived from heart (cardiac) ECM or bone ECM. In particular aspects, the MBV are prepared following digestion of an ECM with an enzyme, such as pepsin, elastase, hyaluronidase, proteinase K, salt solutions, and/or collagenase, or combinations thereof. The ECM can be freeze-thawed, or subject to mechanical degradation.

In some aspects, expression of CD63, CD81, and/or CD9 cannot be detected on the MBV. Thus, in some aspects the MBV do not express CD63 and/or CD81 and/or CD9. In one specific example, CD63, CD81, and CD9 cannot be detected on the nanovesicles. In other aspects, the MBV have barely detectable levels of CD63, CD81, and CD9, such as that detectable by Western blot. These MBV are CD63^{lo}CD81^{lo}CD9^{lo}. In other aspects, MBV do not express detectable levels of one or more of CD63, CD81, or CD9. In other aspects, MBV express barely detectable levels of one or more of CD63, CD81, or CD9. One of skill in the art can readily identify MBV that are CD6310 and/or CD81^{lo} and/or CD910, using, for example, antibodies that specifically bind CD63, CD81, and CD9. A low level of these markers can be established using procedures such as fluorescent activated cell sorting (FACS) and fluorescently labeled antibodies to determine a threshold for low and high amounts of CD63, CD81, and CD9. The disclosed MBV differ from nanovesicles, such as exosomes that may be transiently attached to the surface of the ECM due to their presence in biological fluids, as MBV in vivo are bound to the ECM and not found in biological fluids.

MBV have distinctive phospholipid content, for example, in comparison to exosomes. In some aspects, the total phospholipid content of the MBV is at least 50%, 55%, 60%, 65%, 70%, 75%, 85%, or 90%, or about 50%-90%, 50%-65%, 50%-60%, 50%-70%, 60%-70%, 60%-90%, or 70%-90% of phosphatidylcholine (PC) and phosphatidyl inositol (PI) in combination. In specific aspects, the total phospholipid content of the MBV is at least 55% of phosphatidylcholine (PC) and phosphatidyl inositol (PI) in combination. In specific aspects, the total phospholipid content of the MBV is at least 60% of phosphatidylcholine (PC) and phosphatidyl inositol (PI) in combination. In some aspects, the phospholipid content of the MBV comprises a phosphatidylcholine (PC) to phosphatidyl inositol (PI) ratio of less than 8:1 (for example, less than 7:1, less than 6:1, less than 5:1, less than 4:1, less than 3:1, or less than 2:1). In some aspects, the phospholipid content of the MBV comprises a phosphatidylcholine (PC) to phosphatidyl inositol (PI) ratio in the range of 0.5-1:1, or in the range of 1:0.5-1, or in the range of 0.5-1:2, or in the range of 2:0.5-1, or in the range of 0.8-1:1, or in the range of 1:0.8-1. In one aspect, the phospholipid content of the MBV comprises a phosphatidylcholine (PC) to phosphatidyl inositol (PI) ratio of about 1:1. In specific aspects, the phospholipid content of the MBV comprises a phosphatidylcholine (PC) to phosphatidyl inositol (PI) ratio of about 0.9:1.

In some aspects, the total phospholipid content of the MBV is 15%, 10%, 9%, 8%, 7%, 6%, 5%, 4% or less, or about 5%-10%, 5%-15%, 10%-15%, or 8%-12% of sphingomyelin (SM). In specific aspects, the total phospholipid content of the MBV is 10% or less of sphingomyelin (SM). In some aspects, the total phospholipid content of the is 15% or less of sphingomyelin (SM), 14% or less of sphingomyelin, 13% or less of sphingomyelin, 12% or less of sphingomyelin, 11% or less of sphingomyelin, 10% or less of sphingomyelin, 9% or less of sphingomyelin, 8% or less of sphingomyelin, 7% or less of sphingomyelin, 6% or less of sphingomyelin, 5% or less of sphingomyelin, or 4% or less of sphingomyelin.

In some aspects, the total phospholipid content of the MBV 20%, 19%, 18%, 17%, 16%, 15%, 14%, 13%, 12%, 11%, or 10% or less, or about 10%-20%, 15%-20%, 14%-18%, or 12%-16% of phosphatidylethanolamine (PE). In specific aspects, the total phospholipid content of the MBV is 20% or less of phosphatidylethanolamine (PE).

In some aspects, the total phospholipid content of the MBV is 5%, 10%, 12%, 15%, 18%, 20%, 25%, or 30% or greater, or about 5%-30%, 10%-20%, 10-25%, 15%-25%, or 12%-18% of phosphatidylinositol (PI). In specific aspects, MBV include a phospholipid content 15% or greater of phosphatidylinositol (PI).

In specific aspects, the total phospholipid content of the MBV comprises 15% or more phosphatidylinositol, 20% or less phosphatidylethanolamine, and 10% or less sphingomyelin. In specific aspects, the total phospholipid content of the MBV is 15% or more phosphatidylinositol and 20% or less phosphatidylethanolamine. In specific aspects, the total phospholipid content of the MBV is 15% or more phosphatidylinositol and 10% or less sphingomyelin. In specific aspects, the total phospholipid content of the MBV comprises 20% or less phosphatidylethanolamine and 10% or less sphingomyelin. In specific aspects, the total phospholipid content of the MBV is more than 15% phosphatidylinositol, 20% or less phosphatidylethanolamine, 10% or less sphingomyelin, and at least 55% of phosphatidylinositol and phosphatidylcholine in combination. In one aspect, the total phospholipid content of the MBV is at least 55% phosphatidylcholine (PC) and phosphatidyl inositol (PI) in combination and 10% or less sphingomyelin (SM). In specific aspects, the total phospholipid content of the MBV is at least 55% of phosphatidylinositol and phosphatidylcholine in combination and more than 15% phosphatidylinositol. In specific aspects, the total phospholipid content of the MBV is 55% of phosphatidylinositol and phosphatidylcholine in combination and 20% or less phosphatidylethanolamine.

The MBV may also comprise lysyl oxidase (Lox). Generally, nanovesicles derived from the ECM have a higher Lox content than exosomes. Lox is expressed on the surface of MBV. Nano-LC MS/MS proteomic analysis can be used to detect Lox proteins. Quantification of Lox can be performed (see, e.g., Hill R C, et al., Mol Cell Proteomics. 2015; 14 (4): 961-73, incorporated herein by reference in its entirety).

In certain aspects, the MBV comprise one or more miRNA. In specific non-limiting examples, the MBV comprise one, two, or all three of miR-143, miR-145 and miR-181. MiR-143, miR-145 and miR-181 are known in the art.

The miR-145 nucleic acid sequence is provided in MiRbase Accession No. MI0000461, incorporated herein by reference. A miR-145 nucleic acid sequence is CACCUUGUCCUCACGGUCCAGUUUUCCCAGGAAUCCCUUAGAUGCUAAGAUGGGGAUUCCU GGAAAUACUGUUCUUGAGGUCAUGGUU (SEQ ID NO: 1). An miR-181 nucleic acid sequence is provided in miRbase Accession No. MI0000269, incorporated herein by reference. A miR-181 nucleic acid sequence is:

(SEQ ID NO: 2)
AGAAGGGCUAUCAGGCCAGCCUUCAGAGGACUCCAAGGAACAUUCAAC
GCUGUCGGUGAGUUUGGGAUUUGAAAAAACCACUGACCGUUGACUGUA
CCUUGGGGUCCUUA.

The miR-143 nucleic acid sequence is provided in NCBI Accession No. NR_029684.1, Mar. 30, 2018, incorporated herein by reference. A DNA encoding an miR-143 nucleic acid sequence is: GCGCAGCGCC CTGTCTCCCA GCCTGAGGTG CAGTGCTGCA TCTCTGGTCA GTTGGGAGTC TGAGATGAAG CACTGTAGCT CAGGAAGAGA GAAGTTGTTC TGCAGC (SEQ ID NO: 3).

Following administration, the MBV maintain expression of F4/80 (a macrophage marker) and CD-11b on macrophages in the subject. Nanovesicle treated macrophages are predominantly F4/80+Fizz1+indicating an M2 phenotype.

The MBV disclosed herein can be formulated into compositions for pharmaceutical delivery. MBV are further disclosed and described in PCT Publication No. WO 2017/151862, which is incorporated herein by reference.

Isolation of MBV from the ECM

To produce MBV, ECM can be produced by any cells of interest, or can be utilized from a commercial source, as described supra. The MBV can be produced from the same species as, or a different species than, the subject being treated. In some aspects, these methods include digesting the ECM with an enzyme to produce digested ECM. In specific aspects, the ECM is digested with one or more of pepsin, elastase, hyaluronidase, collagenase a metalloproteinase, and/or proteinase K, or combinations thereof. In a specific non-limiting example, the ECM is digested with only elastase and/or a metalloproteinase. In another non-limiting example, the ECM is not digested with collagenase and/or trypsin and/or proteinase K. In other aspects, the ECM is treated with a detergent. In further aspects, the method does not include the use of enzymes. In specific non-limiting examples, the method utilizes chaotropic agents or ionic strength to isolate MBV such as salts, such as potassium chloride. In additional aspects, the ECM can be manipulated to increase MBV content prior to isolation of MBV. Techniques for isolating MBV from ECM are described, for example, in U.S. Patent Application Publication No. 2019/0117837, the contents of which are incorporated by reference herein for all purposes. Techniques for isolating MBV are also disclosed in Quijano et al., Tissue Eng Part C Methods. 2020 Oct; 26 (10): 528-540, also incorporated by reference herein.

In some aspects, the ECM is digested with an enzyme. The ECM can be digested with the enzyme for about 12 to about 48 hours, such as about 12 to about 36 hours. The ECM can be digested with the enzyme for about 12, about 24 about 36 or about 48 hours. In one specific non-limiting example, the ECM is digested with the enzyme at room temperature. However, the digestion can occur at about 4° C., or any temperature between about 4° C. and 25° C. Generally, the ECM is digested with the enzyme for any length of time, and at any temperature, sufficient to remove collagen fibrils. The digestion process can be varied depending on the tissue source. Optionally, the ECM is processed by freezing and thawing, either before or after digestion with the enzyme. The ECM can be treated with detergents, including ionic and/or non-ionic detergents.

The digested ECM is then processed, such as by centrifugation, to isolate a fibril-free supernatant. In some aspects the digested ECM is centrifuged, for example, for a first step at about 300 to about 1000 g. Thus, the digested ECM can be centrifuged at about 400 g to about 750 g, such as at about 400 g, about 450 g, about 500 g or about 600 g. This centrifugation can occur for about 10 to about 15 minutes, such as for about 10 to about 12 minutes, such as for about 10, about 11, about 12, about 14, about 14, or about 15 minutes. The supernatant including the digested ECM is collected.

In some aspects, the MBV comprise Lox. In some aspects, methods for isolating such MBV include digesting the extracellular matrix with elastase and/or metalloproteinase to produce digested extracellular matrix, centrifuging the digested extracellular matrix to remove collagen fibril remnants and thus to produce a fibril-free supernatant, centrifuging the fibril-free supernatant to isolate the solid materials, and suspending the solid materials in a carrier.

In some aspects, digested ECM also can be centrifuged for a second step at about 2000 g to about 3000 g. Thus, the digested ECM can be centrifuged at about 2,500 g to about 3,000 g, such as at about 2,000 g, 2,500 g, 2,750 g or 3,000 g. This centrifugation can occur for about 20 to about 30 minutes, such as for about 20 to about 25 minutes, such as for about 20, about 21, about 22, about 23, about 24, about 25, about 26, about 27, about 28, about 29 or about 30 minutes. The supernatant including the digested ECM is collected.

In additional aspects, the digested ECM can be centrifuged for a third step at about 10,000 to about 15,000 g. Thus, the digested ECM can be centrifuged at about 10,000 g to about 12,500 g, such as at about 10,000 g, 11,000 g or 12,000 g. This centrifugation can occur for about 25 to about 40 minutes, such as for about 25 to about 30 minutes, for example for about 25, about 26, about 27, about 28, about 29, about 30, about 31, about 32, about 33, about 34, about 35, about 36, about 37, about 38, about 39 or about 40 minutes. The supernatant including the digested ECM is collected. One, two or all three of these centrifugation steps can be independently utilized. In some aspects, all three centrifugation steps are utilized. The centrifugation steps can be repeated, such as 2, 3, 4, or 5 times. In one aspect, all three centrifugation steps are repeated three times.

In some aspects, the digested ECM is centrifuged at about 500 g for about 10 minutes, centrifuged at about 2,500 g for about 20 minutes, and/or centrifuged at about 10,000 g for about 30 minutes. These step(s), such as all three steps are repeated 2, 3, 4, or 5 times, such as three times. Thus, in one non-limiting example, the digested ECM is centrifuged at about 500 g for about 10 minutes, centrifuged at about 2,500 g for about 20 minutes, and centrifuged at about 10,000 g for about 30 minutes. These three steps are repeated three times. Thus, a fibril-free supernatant is produced. The fibril-free supernatant is then centrifuged to isolate the MBV. In some aspects, the fibril-free supernatant is centrifuged at about 100,000 g to about 150,000 g. Thus, the fibril-free supernatant is centrifuged at about 100,000 g to about 125,000 g, such as at about 100,000 g, about 105,000 g, about 110,000 g, about 115,000 g or about 120,000 g. This centrifugation can occur for about 60 to about 90 minutes, such as about 70 to about 80 minutes, for example for about 60, about 65, about 70, about 75, about 80, about 85 or about 90 minutes. In one non-limiting example, the fiber-free supernatant is centrifuged at about 100,000 g for about 70 minutes. The solid material is collected, which is the MBV. These MBV then can be re-suspended in any carrier of interest, such as, but not limited to, a buffer.

In further aspects the ECM is not digested with an enzyme. In these methods, ECM is suspended in an isotonic saline solution, such as phosphate buffered saline. Salt is then added to the suspension so that the final concentration of the salt is greater than about 0.1 M. The concentration can be, for example, up to about 3 M, for example, about 0.1 M salt to about 3 M, or about 0.1 M to about 2M. The salt can be, for example, about 0.1M, 0.15M, 0.2M, 0.3M, 0.4 M, 0.7 M, 0.6 M, 0.7 M, 0.8M, 0.9M, 1.0 M, 1.1 M, 1.2 M, 1.3 M, 1.4 M, 1.5M, 1.6 M, 1.7 M, 1.8M, 1.9 M, or 2M. In some non-limiting examples, the salt is potassium chloride, sodium chloride or magnesium chloride. In other aspects, the salt is sodium chloride, magnesium chloride, sodium iodide, sodium thiocyanate, a sodium salt, a lithium salt, a cesium salt or a calcium salt.

In some aspects, the ECM is suspended in the salt solution for about 10 minutes to about 2 hours, such as about 15 minutes to about 1 hour, about 30 minutes to about 1 hour, or about 45 minutes to about 1 hour. The ECM can be suspended in the salt solution for about 15, 20, 25, 30, 35, 40, 45, 50, 55, 60, 65, 70, 75, 80, 85, 90, 95, 100, 105, 110, 115 or 120 minutes. The ECM can be suspended in the salt solution at temperatures from 4° C. to about 50° C., such as, but not limited to about 4° C. to about 25° C. or about 4° C. to about 37° C. In a specific non-limiting example, the ECM is suspended in the salt solution at about 4° C. In other specific non-limiting examples, the ECM is suspended in the salt solution at about 22° C. or about 25° C. (room temperature). In further non-limiting examples, the ECM is suspended in the salt solution at about 37° C.

In some aspects, the method includes incubating an extracellular matrix at a salt concentration of greater than about 0.4 M; centrifuging the digested extracellular matrix to remove collagen fibril remnants, and isolating the supernatant; centrifuging the supernatant to isolate the solid materials; and suspending the solid materials in a carrier, thereby isolating MBV from the extracellular matrix.

Following incubation in the salt solution, the ECM is centrifuged to remove collagen fibrils. In some aspects, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes. The supernatant is then collected.

In additional aspects, the supernatant then can be centrifuged for a third step at about 100,000 to about 150,000 g. Thus, the digested ECM can be centrifuged at about 100,000 g to about 125,000 g, such as at about 100,000 g, 110,000 g or 120,000 g. This centrifugation can occur for about 30 minutes to about 2.5 hour, such as for about 1 hour to about 3 hours, for example for about 30 minutes, about 45 minutes, about 60 minutes, about 90 minutes, or about 120 minutes (2 hours). The solid materials are collected and suspended in a solution, such as buffered saline, thereby isolating the MBV.

In yet other aspects, the ECM is suspended in an isotonic buffered salt solution, such as, but not limited to, phosphate buffered saline. Centrifugation or other methods can be used to remove large particles (see below). Ultrafiltration is then utilized to isolate MBV from the ECM, particles between about 10 nm and about 10,000 nm, such as between about 10 and about 1,000 nm, such as between about 10 nm and about 300 nm.

In specific non-limiting examples, the isotonic buffered saline solution has a total salt concentration of about 0.164 mM, and a pH of about 7.2 to about 7.4. In some aspects, the isotonic buffered saline solution includes 0.002 M KCl to about 0.164 M KCL, such as about 0.0027 M KCl (the concentration of KCL in phosphate buffered saline). This suspension is then processed by ultracentrifugation.

Following incubation in the isotonic buffered salt solution, the ECM is centrifuged to remove collagen fibrils. In some aspects, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes.

Microfiltration and centrifugation can be used and combined to remove large molecular weight materials from the suspension. In one aspect, large size molecule materials, such as more than 200 nm are removed using microfiltration. In another aspect, large size materials are removed by the use of centrifugation. In a third aspect both microfiltration and ultracentrifugation are used to remove large molecular weight materials. Large molecular weight materials are removed from the suspended ECM, such as materials greater than about 10,000 nm, greater than about 1,000 nm, greater than about 500 nm, or greater than about 300 nm.

The effluent for microfiltration or the supernatant is then subjected to ultrafiltration. Thus, the effluent, which includes particle of less than about 10,000 nm, less than about 1,000 nm, less than about 500 nm, or less than about 300 nm is collected and utilized. This effluent is then subjected to ultrafiltration with a membrane with a molecular weight cutoff (MWCO) of 3,000 to 100,000.

### Preparation of Extracellular Matrix (ECM) Hydrogels

Any type of extracellular matrix can be used to produce a mammalian ECM hydrogel (see U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666 related to ECM). In certain aspects, the ECM is isolated from a vertebrate animal, for example and without limitation, from a mammal including, but not limited to, humans, monkeys, horses, pigs, cows and sheep. In specific non-limiting examples, the ECM is porcine.

ECM can be derived from any organ or tissue, including without limitation, urinary bladder, intestine (such as small intestine or large intestine), heart, kidney, uterus, brain, blood vessel, lung, bone, muscle, pancreas, stomach, spleen adipose tissue, liver, esophagus and dermis. The ECM can be obtained from a cell culture. In one aspect, the ECM is isolated from a urinary bladder. In another aspect, the ECM is from an esophagus. In another aspect, the ECM is from dermis. In another aspect, the ECM is from small intestinal submucosa (SIS). The ECM may or may not include the basement membrane portion of the ECM. In certain aspects, the ECM includes at least a portion of the basement membrane. A tissue can be decellularized to remove cells and cellular material, e.g., from the source tissue or organ, to produce an ECM. It desirable to use a decellularized material prevent an immune response, such as when ECM is implanted in a subject, for example, as a component of a hydrogel disclosed herein. Removal of cellular material, such as when using ECM to form a hydrogel, prevents such an immune response.

U.S. Pat. No. 8,361,503 (incorporated herein by reference in its entirety for all purposes) discloses preparation of a urinary bladder ECM, such as porcine bladder ECM is prepared by abrading bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. In some aspects, perforation of the submucosa is prevented. After these tissues are removed, the resulting ECM consists mainly of the tunica submucosa.

The production of hydrogels from dermal ECM is disclosed in Wolf et al., Biomaterials 33:7028-7038, 2012, incorporated herein by reference. The production of ECM from esophageal tissue is disclosed, for example, in Badylak et al. J Pediatr Surg. 35 (7): 1097-103, 2000 and Badylak et al., J Surg Res. 2005 September; 128 (1): 87-97, 2005, both incorporated herein by reference. U.S. Pat. No. 6,893,666, incorporated herein by reference, discloses production of ECM from urinary bladder, skin, esophagus and small intestine. ECM can be produced from any of these tissues.

Commercially available ECM preparations can also be used. In one aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to, UBM (Acell Corporation; Jessup, Md.).

Tissue for preparation of ECM can be harvested in a large variety of ways and once harvested, a variety of portions of the harvested tissue may be used. ECM has also been prepared from the esophagus and small intestine, see, for example, Keane et al., Tissue Eng. Part A, 21 (17-18): 2293-2300, 2015, incorporated herein by reference. Esophageal ECM can be prepared by mechanically separating the mucosa and submucosa from the muscularis externa and digesting the mucosal layers in a buffer including trypsin, followed by exposure to sucrose, TRITON-X100®, deoxycholic acid, peracetic acid and DNAse. Small intestine submucosa (SIS) can be prepared by mechanically removing the superficial layers of the tunica mucosa, tunica serosa, and tunica muscularis externa from the intact small intestine, leaving the submucosa, muscularis mucosa, and basilar stratum compactum intact. The SIS is then treated with peracetic acid. Exemplary protocols are provided in Keane et al. Dermal hydrogels can be produced, for example, as disclosed in Wolf et al, J Biomed Mater Res A. 2013. 35 (25): 6838-49. PMID: 23873846. PMCID: 3808505, incorporated herein by reference.

In one aspect, the ECM is isolated from harvested porcine urinary bladder to prepare urinary bladder matrix (UBM). Excess connective tissue and residual urine are removed from the urinary bladder. The tunica serosa, tunica muscularis externa, tunica submucosa and most of the muscularis mucosa can be removed by mechanical abrasion or by a combination of enzymatic treatment, hydration, and abrasion. Mechanical removal of these tissues can be accomplished by abrasion using a longitudinal wiping motion to remove the outer layers (particularly the abluminal smooth muscle layers) and even the luminal portions of the tunica mucosa (epithelial layers). Mechanical removal of these tissues is accomplished by removal of mesenteric tissues with, for example, Adson-Brown forceps and Metzenbaum scissors and wiping away the tunica muscularis and tunica submucosa using a longitudinal wiping motion with a scalpel handle or other rigid object wrapped in moistened gauze. The epithelial cells of the tunica mucosa can also be dissociated by soaking the tissue in a de-epithelializing solution, for example and without limitation, hypertonic saline.

The resulting UBM comprises basement membrane of the tunica mucosa and the adjacent tunica propria, which is further treated with peracetic acid, lyophilized and powdered, see U.S. Pat. No. 8,361,503, incorporated herein by reference.

Dermis sections can used for the preparation of the ECM hydrogels, see PCT Application No. 2015/15164728, incorporated herein by reference. In a specific non-limiting example, the dermis can be decellularized with 0.25% Trypsin/1% TRITON-X®-100 (i.e. no SDS) on a vortex shaker at 300 RPM at room temperature in the following solutions: 0.25% trypsin for 6 hours, 1×; deionized water, 15 minutes, 3×; 70% ethanol, 10 to 12 hours, 1×; 3% H₂O₂, 15 minutes, 1×, deionized water, 15 minutes, 2×; 1% TRITON-X®-100 in 0.26% EDTA/0.69% Tris, 6 hours, 1× and then overnight, 1×; deionized water, 15 minutes, 3×; 0.1% peracetic acid/4% ethanol, 2 hours, 1×; PBS, 15 minutes, 2×; and finally deionized water, 15 minutes, 2×. Dermis sheets are then lyophilized and subsequently reduced to particulate form using a Waring blender and a Wiley Mill with a #20 mesh screen.

In some aspects, the epithelial cells can be delaminated first by first soaking the tissue in a de-epithelializing solution such as hypertonic saline, for example and without limitation, 1.0 N saline, for periods of time ranging from 10 minutes to 4 hours. Exposure to hypertonic saline solution effectively removes the epithelial cells from the underlying basement membrane. The tissue remaining after the initial delamination procedure includes epithelial basement membrane and the tissue layers abluminal to the epithelial basement membrane. This tissue is next subjected to further treatment to remove the majority of abluminal tissues but not the epithelial basement membrane. The outer serosal, adventitial, smooth muscle tissues, tunica submucosa and most of the muscularis mucosa are removed from the remaining deepithelialized tissue by mechanical abrasion or by a combination of enzymatic treatment, hydration, and abrasion.

In some aspects, the ECM itself can be sterilized by any number of standard techniques, including, but not limited to, exposure to peracetic acid, low dose gamma radiation, gas plasma sterilization, ethylene oxide treatment or electron beam treatment. More typically, sterilization of ECM is obtained by soaking in 0.1% (v/v) peracetic acid, 4% (v/v) ethanol, and 95.9% (v/v) sterile water for two hours. The peracetic acid residue is removed by washing twice for 15 minutes with PBS (pH=7.4) and twice for 15 minutes with sterile water. ECM material can be sterilized by propylene oxide or ethylene oxide treatment, gamma irradiation treatment (0.05 to 4 mRad), gas plasma sterilization, peracetic acid sterilization, or electron beam treatment. The ECM can also be sterilized by treatment with glutaraldehyde, which causes cross linking of the protein material, but this treatment substantially alters the material such that it is slowly resorbed or not resorbed at all and incites a different type of host remodeling which more closely resembles scar tissue formation or encapsulation rather than constructive remodeling. Cross-linking of the protein material can also be induced with carbodiimide or dehydrothermal or photooxidation methods. As disclosed in U.S. Pat. No. 8,361,503, ECM is disinfected by immersion in 0.1% (v/v) peracetic acid (a), 4% (v/v) ethanol, and 96% (v/v) sterile water for 2 h. The ECM material is then washed twice for 15 min with PBS (pH=7.4) and twice for 15 min with deionized water.

Generally, following isolation of the tissue of interest, decellularization is performed by various methods, for example and without limitation, exposure to hypertonic saline, peracetic acid, TRITON-X® or other detergents. Sterilization and decellularization can be simultaneous. For example and without limitation, sterilization with peracetic acid, described above, also can be used for decellularization. ECM can then be dried, either lyophilized (freeze-dried) or air dried. Dried ECM can be comminuted by methods including, but not limited to, tearing, milling, cutting, grinding, and shearing. The comminuted ECM can also be further processed into a powdered form by methods, for example and without limitation, such as grinding or milling in a frozen or freeze-dried state.

Mammalian ECM is also commercially available. These include AVITENET, MICROMATRIX® and XENMATRIX™. These commercially available products can also be used to produce a mammalian acoustic ECM hydrogel.

### Acoustic ECM Hydrogels

In some aspects, a comminuted ECM, such as a mammalian ECM, is diluted in a liquid for preparation of an acoustic ECM hydrogel. The ECM may or may not be lyophilized prior to comminuting. The ECM can be comminuted, for example, by grinding, chopping or cutting the ECM. Comminuted ECM should have pieces in the range of about 10 μm to about 5000 μm, about 10 μm to about 4000 μm, about 10 μm to about 3000 μm, about 10 μm to about 2000 μm, about 10 μm to about 1000 μm, about 10 μm to about 500 μm, about 30 μm to about 300 μm, about 40 to about 400 μm, about 25 μm to about 500 μm, about 50 μm to about 500 μm, about 100 μm to about 300 μm, about 10 μm to about 50 μm, or about 10 μm to about 100 μm. In one aspect, the ECM is provided in pieces having a range from about 10 μm to about 1000 μm. In another preferred aspect, the ECM is provided in pieces having a range from about 10 μm to about 2000 μm. In one non-limiting example, the pieces are in the range of about 30 μm to about 300 μm. The liquid can be a buffer at neutral pH, such as, for example, a pH of about 7.0 to about 7.6, such as about 7.1 to about 7.5, such as about 7.2 to about 7.4, such as about 7.0 to 7.2, such as about 7.0 to 7.4, such as about 7.1, 7.2, 7.3, 7.4, 7.5 or 7.6. The ECM can be diluted in an isotonic buffered saline solution, such as, but not limited to, phosphate buffered saline (PBS) or Tris buffered saline. In some aspects, the buffered saline solution has an osmolarity of about 290 mOsm/L. The liquid can be water. In some aspects, the isotonic buffer, including, without limitation, Phosphate Buffered Saline (PBS), can be used to bring the solution to a target pH, or to aid in maintaining the pH and ionic strength of the gel to target levels, such as physiological pH and ionic conditions. This forms a liquid ECM solution.

The methods for preparation of an acoustic hydrogel generally do not involve the use of an acid protease, including pepsin, trypsin, or hyaluronidase, or enzymatic digestion of the ECM tissue, generally. See PCT Application No. WO 2015/164728, incorporated herein by reference. Generally, the solubilized ECM in the liquid is not contacted with an acid protease.

Methods of preparing an extracellular matrix hydrogel by application of acoustic techniques, e.g., ultrasonic frequencies, may be found in U.S. Patent Application Publication No. 2022/0143265, the contents of which is incorporated by reference herein for all purposes. In some aspects, the ECM is utilized at a concentration of greater than about 25 mg/ml in the liquid. The ECM can be utilized at a concentration of about 25 mg/ml to about 600 mg/ml in the liquid, such as the buffer. Suitable concentrations also include about 25 mg/ml to about 300 mg/ml, about 25 mg/ml to about 200 mg/ml, and about 25 mg/ml to about 150 mg/ml. The ECM can be utilized at a concentration of about 50 mg/ml to 600 mg/ml in the liquid, such as the buffer. Suitable concentrations also include about 50 mg/ml to about 300 mg/ml, about 50 mg/ml to about 200 mg/ml, and about 50 mg/ml to about 150 mg/ml. Suitable concentrations include about 25, 30, 35, 40, 45, 50, 55, 60, 65, 70, 75, 80, 85, 90, 95, 100, 105, 110, 115, 120, 125, 130, 135, 140, 150, 155, 160, 165, 170, 175, 180, 185, 190, 195, and 200 mg/ml. Exemplary concentrations include about 25 mg/ml, 100 mg/ml, and 150 mg/ml. In one non-limiting example, the ECM in a liquid at a concentration of about 25 mg/ml to about 150 mg/ml. In one non-limiting example, the ECM is in the liquid at a concentration of 100 mg/ml.

The ECM in the liquid, such as the buffered saline solution, is treated with an ultrasound frequency. In one aspect, the ultrasound is at a frequency of about 20 kHz to about 100 kHz. The ECM in the liquid can be treated with ultrasound at a frequency of about 20 kHz to about 30 kHz, about 20 Hz to about 40 kHz, about 20 kHz to about 50 kHz, about 20 kHz to about 60 kHz, about 20 kHz to about 70 kHz, about 20 kHz to about 80 kHz, or about 20 kHz to about 90 kHz. The ECM in the liquid can be treated with ultrasound at a frequency of about 20 kHz, 30 kHz, 40 kHz, 50 kHz, 60 kHz, 70 kHz, 80 kHz, 90 kHz or 100 kHz. In one non-limiting example, the ECM in the liquid can be treated with ultrasound at a frequency of about 20 KHz.

The ECM in the liquid, such as the buffered saline solution, is treated with ultrasound for at least 20 seconds, such as at least 30 seconds. The ECM in the liquid, such as the buffered saline solution, is treated with ultrasound for at least 60 seconds. In some aspects, the ECM in the liquid is treated with ultrasound for at least 60 seconds to about one hour. In further aspects, the ECM in the liquid is treated with ultrasound for at least 60 seconds to about 30 minutes. In further aspects, the ECM in the liquid is treated with ultrasound for at least 30 seconds to about 30 minutes. In more aspects, the ECM in the liquid is treated with ultrasound for at least 60 seconds to about 15 minutes. In more aspects, the ECM in the liquid is treated with ultrasound for at least 30 seconds to about 15 minutes. In some aspects, the ECM in the liquid is treated with ultrasound for at least 60 seconds to about 10 minutes. In some aspects, the ECM in the liquid is treated with ultrasound for at least 30 seconds to about 10 minutes. In some aspects, the ECM in the liquid is treated with ultrasound for at least 60 seconds to about 5 minutes. In some aspects, the ECM in the liquid is treated with ultrasound for at least 30 seconds to about 5 minutes. The ECM in the liquid can treated with ultrasound for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes. In some aspects, the ECM in the liquid is treated with the ultrasound in pulses for a total time as listed herein. Thus, in some aspects, the ECM in the liquid, such as the buffered saline solution, is treated with pulses, such as of at least about 30 seconds in length, such as about 30, about 40 or about 60 seconds in length. The ECM in the liquid such as the buffered saline solution, can be treated 1, 2, 3, 4, 5, 6, 7, 8, 9, 10 times, with the ultrasound, such that the total time of treatment is the 60 seconds to one hour, or any of the total times listed. The ECM in the liquid such as saline solution can be treated for 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 seconds. The ECM in the liquid such as saline solution can be treated for at least 30 seconds. Generally, if multiple treatments are used, they occur in a period of less than 1 hour. An exemplary method is pulses of 30 seconds of ultrasound, followed by no treatment for 30 to 45 seconds, followed by another treatment. This treatment is applied 2, 3, 4, 5, 6, 7, 8, 9 or 10 or more times. One exemplary non-limiting method is six pulses of 30 seconds of ultrasound, such as at about 20 kHz, followed by 45 seconds off, for six repetitions, totaling 3 minutes of treatment with ultrasound.

The ultrasound can have an amplitude of about 20 μm to about 320 μm. Generally, the amplitude is measure from the center of the probe used to produce the ultrasound. The amplitude of the probe's vibrating surface the distance between its position in the probe's fully extended and fully contracted states, measured in microns (μm). In some aspects, the amplitude is about 30 μm to about 200 μm. In further aspects, the amplitude is about 36 μm to about 180 μm. The amplitude can be about 30, 40, 50, 60, 70, 80, 90, 100, 110, 120, 130, 140, 150, 150, 160, 70, 180, 190, 200, 210, 220, 230, 240, 250, 260, 270, 280, 290 or 300 μm. In some aspects, the amplitude can be about 30-40 μm, 40-50 μm, 50-60 μm, 60-70 μm, 70-80 μm, 80-90 μm, 90-100 μm, 100-110, 110-120 μm, 120-130 μm, 130-140 μm, 140-150 μm, 150-160 μm, 160-170 μm, 170-180μ, 180-190μ, 190-200μ, 200-210μ, 210-220 μm 220-230 μm, 230-240 μm, 240-250 μm, 250-260 μm, 260-270 μm, 270-280 μm, 280-290 μm or 290-300 μm. In one specific, non-limiting example, the ultrasound is at a frequency of about 20 kHz, and the amplitude is about 36 μm to about 180 μm. In a further non-limiting example, the ultrasound is at a frequency of about 20 kHz, and the amplitude is about 36 μm to about 180 μm, and the treatment is for a total of about 1, 2, 3, 4, or 5 minutes, such as about 3 minutes. The sonication can be for about 1, 2, 3, 4, 5, 6, 7, 8, 9, 10, 11, 12, 13, 14, 15, 16, 17, 18, 19 or 20 minutes. The sonication can be from about 30 seconds to about 5 minutes. The sonication can be for example, for between about 1 to about 5 minutes. The sonication can be for about 1 to about 10 minutes. The sonication can be, for example, for between 1 to about 20 minutes. In more aspects, the sonication can be for less than about one hour, less than about 30 minutes, less than about 20 minutes, or less than about 10 minutes. In some aspects, the sonication can be for at least 30 seconds. In other aspects, the sonication can be for about 10 minutes to about 24 hours, for example, about 1, 2, 3, 4, 5, 6, 7, 8, 9, 10, 11, 12, 13, 14, 15, 16, 17, 18, 19, 20, 21, 22, 23, or 24 hours. In some aspects, sonication can be for up to 48 hours.

In some aspects, the ECM in the liquid is treated with the ultrasound at a temperature in a range of about 30° C. to about 43° C. In one aspect, the ECM in the liquid is treated with the ultrasound at a temperature in the range of about 35° C. to about 40° C. In one aspect, the ECM in the liquid is treated with ultrasound at a temperature in the range of about 36° C. to about 38° C. In another aspect, the ECM in the liquid is treated with ultrasound at a temperature in the range of about 37° C. or greater, such as a temperature of about 37° C. to about 55° C., such as about 37° C. to about 50° C., such as about 37° C. to about 45° C., such as about 37° C. to about 40° C. The ECM in the liquid is treated with the ultrasound at a temperature of about 37, 38, 39, 40, 41, 42, 43, 44, 45, 46, 47, 48, 49, 50, 51, 52, 53, 54, or 55° C. In further aspects, the ECM in the liquid is treated with the ultrasound at greater than about 38° C., such as about 38° C. to about 50° C., such as about 38° C. to about 45° C., such as about 38° C. to about 40° C.

In aspects, treatment with ultrasound produces an acoustic ECM hydrogel. The acoustic ECM hydrogel generally experiences a phase transition from sol to gel around 37° C. and therefore transitions to a liquid phase at greater than 37° C., and to a gel phase at below 37° C. At 37° C. the acoustic ECM hydrogel is sufficiently viscous to resemble a gel; however, as the temperature is increased above 37° C., the gel transitions to a sol. The acoustic ECM hydrogel forms a gel (sol to gel transition) upon a decrease in temperature below 37° C. Thus, in some aspects, following sonication, the acoustic ECM hydrogel is cooled to a temperature of less than 37° C., such as about 4° C. to about 36° C. The acoustic ECM hydrogel can be cooled to room temperature, which is generally about 25° C. In some aspects, the acoustic ECM hydrogel is cooled to about 15° C. to about 25° C. The acoustic ECM hydrogel can be cooled to about 23° C. to about 27° C. The acoustic ECM hydrogel can be cooled to about 4, 5, 6, 7, 8, 9, 10, 11, 12, 13, 14, 15, 16, 17, 18, 19, 20, 21, 22, 23, 24, 25, 26, 27, 28 29 or 30° C. to induce the gel phase.

In some aspects, exogenous MBV may be added to ECM solution prior to sonication. In yet other aspects, exogenous MBV may be added to the acoustic ECM hydrogel after sonication. The exogenous MBV may be added to the ECM hydrogel before the hydrogel transitions to a gel (e.g., while it is in a liquid phase); therefore, in one aspect, exogenous MBV are added to the acoustic ECM hydrogel at a temperature greater 37° C. to produce a composition comprising an acoustic hydrogel disclosed herein containing exogenous MBV. In another aspect, the exogenous MBV are added to the acoustic ECM hydrogel during its gel phase, e.g., at a temperature lower than 37° C. For example, disclosed herein in is an acoustic ECM hydrogel comprising exogenous MBV.

In some aspects, disclosed is an acoustic mammalian ECM hydrogel, wherein the hydrogel is thermoreversible, wherein the hydrogel is in a solid (gel) phase at temperatures below about 37° C. and is in a liquid (sol) phase at temperatures of greater than 37° C. The acoustic hydrogel can be produced using any of the methods disclosed herein. In some aspects, the storage modulus (G′) is greater than loss modulus (G″) by about an order of magnitude for the acoustic ECM hydrogel. In further aspects, wherein the viscosity of the acoustic ECM hydrogel decreases with increased stress at a temperature of about 15 to about 37° C., such as at about 15, 15, 16, 17, 18, 20, 21, 22, 23, 24, 25, 26, 27, 28, 29, 30, 31, 32, 33, 34, 35, and/or 36° C. In further aspects, the viscosity of the acoustic ECM hydrogel decreases with increased stress at room temperature, and/or at about 23° C. to about 27° C. and/or about 15° C. to about 25° C. In one aspect, the gel to sol transition of the acoustic ECM hydrogel is at about 37° C., such that the hydrogel can be used as a submucosal cushion because it is sufficiently viscous at body temperature.

These acoustic ECM hydrogels can be made from any mammalian ECM disclosed above. In specific, non-limiting example, the ECM is human ECM. In other non-limiting examples, the ECM is urinary bladder ECM, small intestinal submucosal ECM, esophageal EMC, or dermal ECM. In one aspect, the ECM is urinary bladder ECM. In another aspect, the ECM is dermal ECM. In yet another aspect, the ECM is esophageal ECM. The source of ECM may be, for example, porcine, bovine, or ovine.

In some aspects, the acoustic ECM hydrogel includes ECM at a concentration of about 25 mg/ml to about 600 mg/ml. In further aspects, the acoustic ECM hydrogel includes ECM at a concentration of about 20 mg/ml to about 600 mg/ml, about 25 mg/ml to about 300 mg/ml, about 25 mg/ml to about 200 mg/ml, and about 25 mg/ml to about 150 mg/ml. In more aspects, the acoustic ECM hydrogel includes ECM at a concentration of about 50 mg/ml to 600 mg/ml in the liquid, such as in the buffer. The acoustic ECM hydrogel also can have an ECM concentration of about 50 mg/ml to about 300 mg/ml, about 50 mg/ml to about 200 mg/ml, about 50 mg/ml to about 150 mg/ml, about 50-100 mg/ml, or about 100-150 mg/ml. In some non-limiting examples, the acoustic ECM hydrogel includes ECM at a concentration of about 20, 25, 30, 35, 40, 45, 50, 55, 60, 65, 70, 75, 80, 85, 90, 95, 100, 105, 110, 115, 120, 125, 130, 135, 140, 150, 155, 160, 165, 170, 175, 180, 185, 190, 195, and 200 mg/ml. In some non-limiting examples, the acoustic ECM hydrogel includes ECM at a concentration of about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, and 195-200 mg/ml. Exemplary non-limiting concentrations of ECM also include about 25 mg/ml, 100 mg/ml, and 150 mg/ml. In one non-limiting example, the acoustic ECM hydrogel includes ECM at a concentration of about 25 mg/ml to about 150 mg/ml. In one aspect, the ECM concentration is about 100 mg/ml.

In some aspects, the acoustic ECM hydrogel has a viscosity of about 1400 Pas at 15° C., and a viscosity of about 400 Pa*s at a temperature of 25° C., when the concentration of ECM is about 150 mg/mL. In other aspects, the acoustic ECM hydrogel has a storage modulus of approximately 2700 Pas at 15° C., approximately 800 Pa*s at 25° C., and 600 Pa*s at 37° C., when the concentration of ECM is about 150 mg/mL.

The acoustic ECM hydrogel in the liquid phase, can be placed into a three-dimensional cast prior to cooling, or spread on a TEFLON® sheet to form a film. The high concentration of ECM in (50 to 600 mg/ml) in the acoustic ECM hydrogel allows for the formation of very thin sheets, for example a sheet as thin as 4 microns. The acoustic ECM hydrogel can be configured to any size greater than 4 microns and in any 2-dimensional or 3-dimensional shape. In some aspects, a sheet is formed that is about 4 to about 10 microns in thickness, such as about 4, 5, 6, 7, 8, 9, or 10 microns in thickness. The acoustic ECM hydrogel can be formed into any three-dimensional shape, which includes, without limitation a cylinder, sphere, ellipsoid, disk, sheet, cube, cuboid, cone, triangular or rectangular prism, as well as hollow spheres, hollow ellipsoids, and open-ended hollow cylinders, etc. The acoustic ECM hydrogel can also be used as an injectable, such as by placing it in a syringe and extruding it from the syringe in either a gel or sol phase.

In some aspects, the mammalian acoustic ECM hydrogel includes solubilized ECM at a concentration of greater than about 0.1 mg/ml. The mammalian acoustic ECM hydrogel can include solubilized ECM at a concentration of about 0.1 mg/ml to about 1,000 mg/ml. Suitable concentrations also include about 1 mg/ml to about 1,000 mg/ml, 1 mg/ml to about 500 mg/ml, 1 mg/ml to about 300 mg/ml, 1 mg/ml to about 200 mg/ml, about 1 mg/ml to about 100 mg/ml, about 10 mg/ml to 100 mg/ml, about 10 mg/ml to about 200 mg/ml, about 100 mg/ml to about 500 mg/ml, about 50 mg/ml to about 150 mg/ml, about 20 mg/ml to about 70 mg/ml, about 4 mg/ml to about 20 mg/ml, or about 40 mg/ml to about 66 mg/ml of solubilized ECM. The mammalian acoustic ECM hydrogel can include solubilized ECM at a concentration of about 10 mg/ml to about 500 mg/ml in the liquid, such as the buffer. The mammalian acoustic ECM hydrogel can include 10, 25, 30, 35, 40, 45, 50, 55, 60, 65, 70, 75, 80, 85, 90, 95, 100, 105, 110, 115, 120, 125, 130, 135, 140, 150, 155, 160, 165, 170, 175, 180, 185, 190, 195, and 200 mg/ml solubilized ECM. Exemplary concentrations include about 20 mg/ml, 40 mg/ml, 66 mg/ml, 70 mg/ml, and 150 mg/ml solubilized ECM. In one non-limiting example, the mammalian acoustic ECM hydrogel includes solubilized ECM at a concentration of about 20 mg/ml to about 70 mg/ml. In one non-limiting example, the mammalian acoustic ECM hydrogel includes solubilized ECM a concentration of about 40 mg/ml or about 66 mg/ml. In one non-limiting example, the mammalian acoustic ECM hydrogel includes solubilized ECM at a concentration of about 10 mg/ml to about 100 mg/ml. In one non-limiting example, the mammalian acoustic ECM hydrogel includes solubilized ECM at a concentration of about 50 mg/ml to about 150 mg/ml. In one non-limiting example, the mammalian acoustic ECM hydrogel includes solubilized ECM at a concentration of about 10 mg/ml to about 200 mg/ml. In one non-limiting example, the mammalian acoustic ECM hydrogel includes solubilized ECM at a concentration of about 10 mg/ml to about 500 mg/ml.

Exemplary concentrations include about 20 mg/ml, 40 mg/ml, 66 mg/ml, 70 mg/ml, and 150 mg/ml of solubilized ECM. In one non-limiting example, the mammalian acoustic ECM hydrogel includes about 20 mg/ml to about 70 mg/ml solubilized ECM. In one non-limiting example, the mammalian acoustic ECM hydrogel includes about 40 mg/ml or about 66 mg/ml of solubilized ECM.

In some aspects, the mammalian acoustic ECM hydrogel includes solubilized ECM at a concentration of about 25 mg/ml to about 600 mg/ml. In further aspects, the mammalian acoustic ECM hydrogel includes solubilized ECM at a concentration of about 20 mg/ml to about 600 mg/ml, about 25 to about 500 mg/ml, about 25 to about 400 mg/ml, about 25 mg/ml to about 300 mg/ml, about 25 mg/ml to about 200 mg/ml, and about 25 mg/ml to about 150 mg/ml. In more aspects, the mammalian acoustic ECM hydrogel includes solubilized ECM at a concentration of about 50 mg/ml to 600 mg/ml. The mammalian acoustic ECM hydrogel also can include solubilized ECM at a concentration of about 50 mg/ml to about 300 mg/ml, about 50 mg/ml to about 200 mg/ml, about 50 mg/ml to about 150 mg/ml, about 50-100 mg/ml, or about 100-150 mg/ml. In some non-limiting examples, the mammalian acoustic ECM hydrogel includes solubilized ECM at a concentration of about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, and 195-200 mg/ml

In some aspects, a composition is produced that includes the mammalian acoustic ECM hydrogel and trehalose. In more aspects, a composition is used includes about 0.1 mg/ml to about 700 mg/ml of trehalose. In some aspects, the composition includes about 1 mg/ml trehalose to about 700 mg/ml trehalose. In further aspects, the composition includes 50 mg/ml to about 500 mg/ml trchalosc. In other aspects, the composition includes about 10 mg/ml trehalose to about 600 mg/ml, about 10 mg/ml to about 500 mg/ml, about 10 mg/ml to about 400 mg/ml, about 10 mg/ml to about 300 mg/ml, about 10 mg/ml to about 200 mg/ml, or about 10 mg/ml to about 100 mg/ml trehalose. In further aspects, the composition can include about 0.1 to about 100 mg/ml trehalose, about 0.1 to about 10 mg/ml trehalose, or about 0.1 to about 1 mg/ml trehalose. In more aspects, the composition can include about 50 mg/ml to about 400 mg/ml trehalose, about 50 mg/ml to about 300 mg/ml trehalose, about 50 mg/ml to about 200 mg/ml trehalose, or about 50 ml/ml to about 100 mg/ml trehalose. In some aspects, the composition includes about 20 mg/ml to about 70 mg/ml trehalose. In some aspects, the composition includes about 10 mg/ml to about 100 mg/ml trehalose. In some aspects, the composition includes 15-30 mg/ml trehalose. In some aspects, the composition includes 60-70 mg/ml trehalose. In some aspects, the composition includes 20 mg/ml trehalose. In some aspects, the composition includes 66 mg/ml trehalose. In other aspects, the composition can include about 1, 2, 3, 4, 5, 6, 7, 8, 9, 10, 20, 30, 40, 50, 60, 66, 70, 80, 90, 100, 200, 300, 400, 500, or 600 mg/ml of trehalose. In other aspects, the composition includes about 100 mg/ml to about 700 mg/ml trehalose, such as about 100, 150, 20, 250, 300, 350, 400, 450, 500, 550, or 600 mg·ml trehalose. In more aspects, the composition can include about 0.1, 0.2, 0.3, 0.4, 0.5, 0.6, 0.7, 0.8, 0.9 or 1 mg/ml trehalose. In more aspects, the composition includes the mammalian acoustic ECM hydrogel comprising solubilized ECM, additional comminuted mammalian ECM, and optionally trehalose. Comminuted ECM is not treated with ultrasound, and is not solubilized into the hydrogel. The comminuted ECM is a distinct additive to composition that also includes the mammalian ECM hydrogel. The composition can include about 1 to about 30% comminuted ECM, weight per volume (w/v), that is not solubilized in the acoustic ECM hydrogel. Without being bound by theory, comminuted ECM generally has intact collagen particles, whereas an acoustic ECM hydrogel has collagen that has been disrupted by ultrasound resulting in an increase in soluble collagen content (Hussey et al., Ultrasonic cavitation to prepare ECM hydrogels Acta Biomater. 2020 May; 10⁸:77-86, incorporated herein by reference in its entirety). As such, an acoustic ECM hydrogel composition containing additional comminuted mammalian ECM includes both intact collagen and disrupted collagen.

The composition can include about 5% to about 30% w/v, about 10% to about 30%, about 15% to about 30%, about 20% to about 30%, about 25% to about 30%, about 1% to about 20%, about 5% to about 20%, about 10% to about 20%, about 15% to about 20%, about 10% to about 20%, or about 15% to about 20% comminuted ECM (w/v). The composition can include about 1, 2, 3, 4, 5, 6, 7, 8, 9, 10, 15, 20, 25, or 30% comminuted ECM (w/v). The composition can include no more than 1, 2, 3, 4, 5, 6, 7, 8, 9, 10, 15, 20, 25, or 30% comminuted ECM (w/v). The composition can include at least about 1, 2, 3, 4, 5, 6, 7, 8, 9, 10, 15, 20, 25, or 30% comminuted ECM (w/v).

Comminuted ECM can be from the same species as the mammalian acoustic ECM hydrogel. In one specific non-limiting example, both the mammalian acoustic ECM hydrogel and the comminuted ECM are porcine. In other non-limiting examples, both the mammalian acoustic ECM hydrogel and the comminuted ECM are human.

The comminuted ECM can be from the same or different tissue as the mammalian acoustic ECM hydrogel. In one aspect, the mammalian acoustic ECM hydrogel and the comminuted ECM are from the same tissue. In one aspect, the mammalian acoustic ECM hydrogel and the comminuted ECM are dermal ECM. In one aspect, the mammalian acoustic ECM hydrogel and the comminuted ECM are porcine dermal ECM.

The composition can be sterilized prior to application to a subject. The composition can be sterilized using any methods known to those of skill in the art, including filtration and radiation. In some aspects, the composition is sterilized with ionizing radiation, such as e-beam or gamma radiation. The composition can be sterilized using gamma radiation, for example, the composition is sterilized using 10 to 50 kGy irradiation, such as 15 to 45 kGy irradiation, 20 to 40 kGy irradiation, or 10 to 30 kGy of irradiation.

In some non-limiting examples, the composition is sterilized using 10, 15, 20, 25, 30, 35, 40, 45 or 50 kGy irradiation. Generally, the composition is sterilized for a sufficient time to achieve an absence of detectable viable pathogens, such as, but not limited to, viruses and bacteria.

### Enzymatic ECM Hydrogels

Methods of preparing ECM hydrogels, are disclosed for example, in U.S. Pat. No. 8,361,503, the contents of which is incorporated by reference herein for all purposes. Any type of extracellular matrix tissue can be used to produce a hydrogel which can be used in the methods as disclosed herein (see U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666 related to ECM). In certain aspects, the ECM is isolated from a vertebrate animal, for example and without limitation, from a warm blooded mammalian vertebrate animal including, but not limited to, humans, monkeys, horses, pigs, cows and sheep. In specific non-limiting examples, the ECM is porcine or human.

The ECM can be derived from any organ or tissue, including without limitation, urinary bladder, intestine, liver, esophagus and dermis. For example, the ECM can be derived from urinary bladder, small intestine, heart, dermis, liver, kidney, uterus, brain, blood vessel, lung, bone, muscle, pancreas, placenta, stomach, spleen, colon, adipose tissue, and/or esophagus. The ECM can be obtained from a cell culture. In one aspect, the ECM is isolated from a urinary bladder. In another aspect, the ECM is from an esophagus. The ECM may or may not include the basement membrane portion of the ECM. In certain aspects, the ECM includes at least a portion of the basement membrane.

In some aspects, as U.S. Pat. No. 8,361,503 (incorporated herein by reference), a urinary bladder ECM, such as porcine bladder ECM is prepared by abrading bladder tissue to remove the outer layers including both the tunica serosa and the tunica muscularis using a longitudinal wiping motion with a scalpel handle and moistened gauzc. Following cversion of the tissue segment, the luminal portion of the tunica mucosa is delaminated from the underlying tissue using the same wiping motion. In some aspects, perforation of the submucosa is prevented. After these tissues are removed, the resulting ECM consists mainly of the tunica submucosa. The production of hydrogels from decellularized dermal ECM is disclosed in Wolf et al., Biomaterials 33:7028-7038, 2012, incorporated herein by reference. The production of ECM from esophageal tissue is disclosed, for example, in Badylak et al. J Pediatr Surg. 35 (7): 1097-103, 2000 and Badylak et al., J Surg Res. 2005 September; 128 (1): 87-97, 2005, both incorporated herein by reference. U.S. Pat. No. 6,893,666, incorporated herein by reference, discloses production of ECM from urinary bladder, skin, esophagus and small intestine.

Commercially available ECM preparations can also be used in the methods, devices and compositions described herein. In one aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to UBM (Acell Corporation; Jessup, Md.).

Tissue for preparation of ECM can be harvested in a large variety of ways and once harvested, a variety of portions of the harvested tissue may be used. ECM has also been prepared from the esophagus and small intestine, and hydrogels have been prepared from this ECM, see, for example, Keane et al., Tissue Eng. Part A, 21 (17-18): 2293-2300, 2015, incorporated herein by reference. Esophageal ECM can be prepared by mechanically separating the mucosa and submucosa from the muscularis externa and digesting the mucosal layers in a buffer including trypsin, followed by exposure to sucrose, TRITON-X100®, deoxycholic acid, peracetic acid and DNAse. Small intestine submucosa (SIS) can be prepared by mechanically removing the superficial layers of the tunica mucosa, tunica serosa, and tunica muscularis externa from the intact small intestine, leaving the submucosa, muscularis mucosa, and basilar stratum compactum intact. The SIS is then treated with peracetic acid. Exemplary protocols are provided in Keane et al. Dermal hydrogels can be produced, for example, as disclosed in Wolf et al, J Biomed Mater Res A. 2013. 35 (25): 6838-49. PMID: 23873846. PMCID: 3808505, incorporated herein by reference.

In one aspect, the ECM is isolated from harvested porcine urinary bladder to prepare urinary bladder matrix (UBM). Excess connective tissue and residual urine are removed from the urinary bladder. The tunica serosa, tunica muscularis externa, tunica submucosa and most of the muscularis mucosa can be removed by mechanical abrasion or by a combination of enzymatic treatment, hydration, and abrasion. Mechanical removal of these tissues can be accomplished by abrasion using a longitudinal wiping motion to remove the outer layers (particularly the abluminal smooth muscle layers) and even the luminal portions of the tunica mucosa (epithelial layers). Mechanical removal of these tissues is accomplished by removal of mesenteric tissues with, for example, Adson-Brown forceps and Metzenbaum scissors and wiping away the tunica muscularis and tunica submucosa using a longitudinal wiping motion with a scalpel handle or other rigid object wrapped in moistened gauze. The epithelial cells of the tunica mucosa can also be dissociated by soaking the tissue in a de-epithelializing solution, for example and without limitation, hypertonic saline. The resulting UBM comprises basement membrane of the tunica mucosa and the adjacent tunica propria, which is further treated with peracetic acid, lyophilized and powdered, see U.S. Pat. No. 8,361,503, incorporated herein by reference.

Dermis sections can used for the preparation of the enzymatic ECM hydrogels, see PCT Application No. 2015/15164728, incorporated herein by reference. In a specific non-limiting example, the dermis can be decellularized with 0.25% Trypsin/1% Triton X-100 (i.e. no SDS) on a vortex shaker at 300 RPM at room temperature in the following solutions: 0.25% trypsin for 6 hours, 1×; deionized water, 15 minutes, 3×; 70% ethanol, 10 to 12 hours, 1×; 3% H₂O₂, 15 minutes, 1×, deionized water, 15 minutes, 2×; 1% Triton X-100 in 0.26% EDTA/0.69% Tris, 6 hours, 1× and then overnight, 1×; deionized water, 15 minutes, 3×; 0.1% peracetic acid/4% ethanol, 2 hours, 1×; PBS, 15 minutes, 2×; and finally deionized water, 15 minutes, 2×. Dermis sheets are then lyophilized and subsequently reduced to particulate form using a Waring blender and a Wiley Mill with a #20 mesh screen.

In some aspects, the epithelial cells can be delaminated first, by first soaking the tissue in a de-epithelializing solution such as hypertonic saline, for example and without limitation, 1.0 N saline, for periods of time ranging from 10 minutes to 4 hours. Exposure to hypertonic saline solution effectively removes the epithelial cells from the underlying basement membrane. The tissue remaining after the initial delamination procedure includes epithelial basement membrane and the tissue layers abluminal to the epithelial basement membrane. This tissue is next subjected to further treatment to remove the majority of abluminal tissues but not the epithelial basement membrane. The outer serosal, adventitial, smooth muscle tissues, tunica submucosa and most of the muscularis mucosa are removed from the remaining deepithelialized tissue by mechanical abrasion or by a combination of enzymatic treatment, hydration, and abrasion.

ECM can be sterilized by any number of standard techniques, including, but not limited to, exposure to peracetic acid, low dose gamma radiation, gas plasma sterilization, ethylene oxide treatment or electron beam treatment. More typically, sterilization of ECM is obtained by soaking in 0.1% (v/v) peracetic acid, 4% (v/v) ethanol, and 95.9% (v/v) sterile water for two hours. The peracetic acid residue is removed by washing twice for 15 minutes with PBS (pH=7.4) and twice for 15 minutes with sterile water. ECM material can be sterilized by propylene oxide or ethylene oxide treatment, gamma irradiation treatment (0.05 to 4 mRad), gas plasma sterilization, peracetic acid sterilization, or electron beam treatment. The ECM can also be sterilized by treatment with glutaraldehyde, which causes cross-linking of the protein material, but this treatment substantially alters the material such that it is slowly resorbed or not resorbed at all and incites a different type of host remodeling which more closely resembles scar tissue formation or encapsulation rather than constructive remodeling. Cross-linking of the protein material can also be induced with carbodiimide or dehydrothermal or photooxidation methods. As disclosed in U.S. Pat. No. 8,361,503, ECM is disinfected by immersion in 0.1% (v/v) peracetic acid (a), 4% (v/v) ethanol, and 96% (v/v) sterile water for 2 h. The ECM material is then washed twice for 15 min with PBS (pH=7.4) and twice for 15 min with deionized water.

Following isolation of the tissue of interest, decellularization is performed by various methods, for example and without limitation, exposure to hypertonic saline, peracetic acid, TRITON-X® or other detergents. Sterilization and decellularization can be simultaneous. For example, and without limitation, sterilization with peracetic acid, described above, also can serve to decellularize the ECM. Decellularized ECM can then be dried, either lyophilized (freeze-dried) or air dried. Dried ECM can be comminuted by methods including, but not limited to, tearing, milling, cutting, grinding, and shearing. The comminuted ECM can also be further processed into a powdered form by methods, for example and without limitation, such as grinding or milling in a frozen or freeze-dried state. In order to prepare solubilized ECM tissue, comminuted ECM is digested with an acid protease in an acidic solution to form a digest solution. The acid protease may be trypsin and/or pepsin, for example, or a combination thereof.

In one aspect, the decellularized ECM material is partially digested by the acid protease. In one example, the decellularized ECM material is digested less completely than a digestion of 1 mg/mL lyophilized, powdered ECM material with 1 mg/mL pepsin in 0.01 M HCl for 48 hours. In another example, the decellularized ECM material is digested less completely than a digestion of 10 mg/mL lyophilized, powdered ECM material with 1 mg/mL pepsin in 0.01 M HCl for 48 hours. In one further aspect, hyaluronic acid in the ECM material is digested less than 50%, 40%, 30%, 25%, 20% or 10% as compared to undigested ECM material, see PCT Application No. WO 2015/164728, incorporated herein by reference.

The digest solution of ECM typically is kept at a constant stir for a certain amount of time at room temperature. The ECM digest can be used immediately or be stored at −20° C. or frozen at, for example and without limitation, −20° C. or −80° C. Thus, the ECM digest can be kept in a solubilized form. Methods for keeping a hydrogel in a solubilized form are disclosed, for example, in PCT Application No. PCT/US16/52261, filed Sep. 10, 2016, incorporated herein by reference.

Once the ECM is solubilized (typically substantially completely) the pH of the solution is raised to between 7.2 and 7.8, and according to one aspect, to pH 7.4. The pH can be raised to about 7.2, 7.3, 7.4, 7.5, 7.6, 7.7 or 7.8. Bases, such as bases containing hydroxyl ions, including NaOH, can be used to raise the pH of the solution. Likewise buffers, such as an isotonic buffer, including, without limitation, Phosphate Buffered Saline (PBS), can be used to bring the solution to a target pH, or to aid in maintaining the pH and ionic strength of the gel to target levels, such as physiological pH and ionic conditions. This forms a “pre-gel” solution which is a solubilized ECM hydrogel. The neutralized digest solution (pre-gel, solubilized ECM hydrogel) can be gelled at lower critical solution temperature, see PCT Publication No. 2015/164728, incorporated herein by reference.

The ECM hydrogel forms a gel (sol to gel transition) upon an increase in temperature. The lower critical solution temperature (LCST) in a reverse gel is a temperature below which a reverse-gelling polymer is soluble in its solvent (e.g. water or an aqueous solvent). As the temperature rises above the LOST in a reverse gel, a hydrogel is formed. The general concept of reverse gelation of polymers and its relation to LCST are broadly known in the chemical arts. The ECM gels described herein are prepared, for example from decellularized, intact ECM as described below, by digestion of the ECM material with an acid protease, neutralization of the material to form a pre-gel, raising the temperature of the pre-gel above the LCST of the pre-gel to cause the pre-gel to gel, such as to form a hydrogel. The transition temperature for acid-protease-digested from solution to gel is typically within the range of from 10° C. to 40° C. and any increments or ranges there between, for example from 20° C. to 35° C. For example, the pre-gel can be warmed to 37° C. to form a hydrogel. For example, the pre-gel.

Thus, the ECM typically can be derived from mammalian tissue, such as, without limitation from one of urinary bladder, esophagus, or small intestine. In one specific non-limiting example, the ECM is derived from urinal bladder. According to one aspect, the decellularized ECM material prepared from the tissue is not dialyzed prior to the partial or complete digestion with the acid protease and/or is not dialyzed after digesting with an acid protease and before gelling of the neutralized, digested ECM material.

In one non-limiting aspect, the ECM is lyophilized and comminuted. The ECM is then solubilized with an acid protease in an acidic solution to produce digested ECM, such as urinary bladder ECM. The acid protease may be, without limitation, pepsin or trypsin, or a combination thereof. The ECM can then be solubilized at an acid pH suitable or optimal for the protease, such as greater than about pH 2, or between pH and 4, for example in a 0.01M HCl solution. The ECM is typically is solubilized for about 12 to about 48 hours, depending upon the tissue type (e.g., see examples below), with mixing (stirring, agitation, admixing, blending, rotating, tilting, etc.). ECM hydrogel is prepared by (i) comminuting an extracellular matrix, (ii) solubilizing intact, non-dialyzed or non-cross-linked extracellular matrix by digestion with an acid protease in an acidic solution to produce a digest solution, (iii) raising the pH of the digest solution to a pH between 7.2 and 7.8 to produce a neutralized digest solution (pre-gel solution), and (iv) gelling the solution.

Accordingly, disclosed is a composition of enzymatically digested ECM in an acid solution with an acid protease and containing exogenous MBV. When, neutralized, e.g., to pH 7.0-7.8, and when warmed to about 37° C., the composition forms a gel and the proteases are inactivated. In one aspect, the exogenous MBV are not derived from bone or cardiac tissue. In a further aspect, the concentration of exogenous MBV in the composition is greater than 5 mg/mL.

Also disclosed is a composition of an enzymatically digested ECM in a neutral solution, e.g., pH 7.0-7.8, where the solution contains inactivated acid proteases, e.g., inactivated pepsin and/or trypsin, or another inactivated acid protease that in its active form is suitable for digesting ECM; the composition also contains exogenous MBV. The solution when warmed to about 37° C. forms a gel. In one aspect, the exogenous MBV are not derived from bone or cardiac tissue. In a further aspect, the concentration of exogenous MBV in the composition is greater than 5 mg/mL. Acid proteases can be inactivated or deactivated due to, e.g., pH changes.

In a further aspect, the ECM hydrogel can be centrifuged, and a soluble fraction is collected. Exemplary methods for fractionation of an ECM hydrogel are disclosed, for example, in PCT Publication No. WO 2015/164728, incorporated herein by reference. The methods disclosed in this PCT publication include partially or completely digesting with an acid protease, such as pepsin, decellularized ECM material prepared from a tissue; neutralizing the digested ECM material to a pH of 7.0-8.0, 7.2-7.8 or 7.4; gelling the neutralized, digested ECM material at a temperature above its Lower Critical Solution Temperature; centrifuging the gelled ECM material to produce a pellet and a supernatant; and separating the supernatant and the pellet thereby separating a structural and a soluble fraction of the ECM material.

The ECM hydrogel, when exposed to temperatures above the Lower Critical Solution Temperature, such as a temperature of about 37° C., forms the gel. The ECM hydrogel in the “pre-gel” form (the solubilized ECM hydrogel), can be frozen and stored at, for example and without limitation, −20° C. or −80° C. The ECM hydrogel in the “pre-gel” form can be stored at room temperature, such about 25° C. In some non-limiting examples, the ECM hydrogel is in the pre-gel form at below 37° C., such as at 25, 24, 23, 22, 21, 20, 19, 18, 17, 16, 15, 14, 13, 12, 11, 10, 9, 8, 7, 6, 5, 4° C. The ECM hydrogel can be frozen for storage, and thus, can be stored at below 0° C. As used herein, the term “pre-gel form” or “pre-gel” refers to the ECM hydrogel wherein the pH is increased, but has not gelled. For example, and without limitation, an ECM hydrogel in the pre-gel form has a pH between 7.2 and 7.8. In some aspects, the solubilized ECM hydrogel is used in the methods disclosed herein. Methods for keeping a hydrogel in a solubilized form are disclosed, for example, in PCT Application No. PCT/US16/52261, filed Sep. 10, 2016, incorporated herein by reference. In some aspects, the ECM composition prepared by any method described herein is absorbed into, adsorbed onto, or otherwise dispersed onto or into a biocompatible substrate. Non-limiting examples of a biocompatible substrate include: a mesh, a non-woven, decellularized tissue, a polymer composition, a polymeric structure, a cell growth scaffold, an implant, an orthopedic implant, and intraocular lens, sutures, intravascular implants, stents, and transplants. The compositions described herein can be applied to or incorporated into, by any suitable method, a non-woven material, such as a bandage, a suture, an implant, such as a ceramic, metal, or polymeric implant, for example a prosthesis, artificial or otherwise-modified vessel, a valve, an intraocular lens, or a tissue implant. As used herein, the term “coat”, and related cognates such as “coated” and “coating,” refers to a process comprising of covering, in part or in whole, an inorganic structure with a composition described herein. For example and without limitation, coating of an inorganic structure with solubilized fraction can include methods such as pouring, embedding, layering, dipping, spraying. Ultrasonication may be used to aid in coating of an inorganic structure.

Compositions of use include ECM hydrogels that are “enzymatic” ECM hydrogels containing exogenous MBV. Exogenous MBV are added to enrich the bioactive properties of the ECM hydrogels and improve their therapeutic efficacy in reducing inflammation and enhancing tissue growth and repair when administered to or implanted in a subject. Enzymatic ECM hydrogels are made from solubilized ECM. In order to prepare solubilized ECM tissue, comminuted ECM is digested with an acid protease in an acidic solution to form a digest solution. As used herein, the term “acid protease” refers to an enzyme that cleaves peptide bonds, wherein the enzyme has increased activity of cleaving peptide bonds in an acidic pH. For example and without limitation, acid proteases can include pepsin and trypsin. In one aspect, the ECM is lyophilized prior to comminution.

The digest solution of ECM typically is kept at a constant stir for a certain amount of time at room temperature. The ECM digest can be used immediately or be stored at −20° C. or frozen at, for example and without limitation, −20° C. or −80° C. To form a “pre-gel” solution, the pH of the digest solution is raised to a pH between 7.2 and 7.8. The pH can be raised by adding one or more of a base or an isotonic buffered solution, for example and without limitation, NaOH or PBS at pH 7.4. The method typically does not include a dialysis step prior to gelation, yielding a more-complete ECM-like matrix that typically gels at 37° C. more slowly than comparable collagen or dialyzed ECM preparations. The gel is therefore more amenable to injection into a patient, and also retains more of the qualities of native ECM due to retention of many native soluble factors, such as, without limitation, cytokines.

As used herein, the term “isotonic buffered solution” refers to a solution that is buffered to a pH between 7.2 and 7.8 and that has a balanced concentration of salts to promote an isotonic environment. As used herein, the term “base” refers to any compound or a solution of a compound with a pH greater than 7. For example and without limitation, the base is an alkaline hydroxide or an aqueous solution of an alkaline hydroxide. In certain aspects, the base is NaOH or NaOH in PBS.

This “pre-gel” solution can, at that point be incubated at a suitably warm temperature, for example and without limitation, at about 37° C. to gel. The pre-gel can be frozen and stored at, for example and without limitation, −20° C. or −80° C. As used herein, the term “pre-gel solution” or” pre-gel” refers to a digest solution wherein the pH is increased. For example and without limitation, a pre-gel has a pH between 7.2 and 7.8. The ECM hydrogel compositions of the invention may include inactivated acid protease. The ECM hydrogel compositions of the invention may have a pH between 7.2 and 7.8. The “pre-gel” may contain exogenous MBV. In one aspect, the exogenous MBV are not derived from cardiac or bone ECM.

The ECM hydrogel, the digest solution or pregel may contain solubilized ECM at a concentration of between 1 mg/mL and 500 mg/mL. In some aspects, the amount of solubilized ECM in the ECM hydrogel, the digest solution or pregel is between 1 mg/mL and 400 mg/mL, e.g., 1 mg/mL to 350 mg/mL, or 1 mg/mL to 300 mg/mL, or 1 mg/mL to 250 mg/mL or 1 mg/mL to 200 mg/mL or 1 mg/mL to 150 mg/mL or 1 mg/mL to 100 mg/mL or 1 mg/mL to 50 mg/mL, or 5 mg/mL to 250 mg/mL or 20 mg/mL to 200 mg/mL or 5 mg/mL to 200 mg/mL or 5 mg/mL to 100 mg/mL. In more aspects, the amount of solubilized ECM in the ECM hydrogel, the digest solution or pregel is between about 5 mg/ml to about 50 mg/ml, such as about 10 mg/ml to about 50 mg/ml, about 20 mg/ml to about 50 mg/ml, about such as about 30 mg/ml to about 50 mg/ml, about 40 mg/ml to about 50 mg/ml, about 5 mg/ml to about 40 mg/ml, about 5 mg/ml to about 30 mg/ml, about 5 mg/ml to about 20 mg/mg, or about 5 mg/ml to about 10 mg/ml. For example, the ECM hydrogel, the digest solution or pregel may contain solubilized ECM at a concentration of about 5, 10, 15, 20, 25, 30, 35, 40, 45, 50, 60, 65, 70, 75, 80, 85, 90, 95, 100, 150, 200, 250, 300, 350, 400, 450, or 500 mg/ml. In one non-limiting example, the amount of solubilized ECM in the ECM hydrogel, the digest solution or pregel is between 10 mg/mL and 30 mg/mL. In another non-limiting example, the amount of solubilized ECM in ECM hydrogel, the digest solution or pregel is between 1 mg/mL and 20 mg/mL. In yet another non-limiting example, the amount of solubilized ECM in the ECM hydrogel, the digest solution or pregel is between 4 mg/mL and 20 mg/mL. In another non-limiting example, the amount of solubilized ECM in the ECM hydrogel, the digest solution or pregel is between 1 mg/mL and 50 mg/mL. In some aspects, the amount of solubilized ECM in the ECM hydrogel, the digest solution or pregel is about 5 to about 100 mg/mL, e.g., 50 to 100 mg/mL, 25 to 75 mg/mL, 60 to 80 mg/mL, 40 to 60 mg/mL, 50 to 80 mg/mL, or about 30 to about 60 mg/mL.

### Additional Considerations

In some aspects, a disclosed composition is absorbed into, adsorbed onto, or otherwise dispersed onto or into a biocompatible substrate. Non-limiting examples of a biocompatible substrate include: a mesh, a non-woven, decellularized tissue, a polymer composition, a polymeric structure, a cell growth scaffold, an implant, an orthopedic implant, and intraocular lens, sutures, intravascular implants, stents, and transplants. In some aspects, the substrate is synthetic. In other aspects, the substrate is natural. The disclosed composition can be applied to or incorporated into, by any suitable method, a non-woven material, such as a bandage, a suture, an implant, such as a ceramic, metal, or polymeric implant, for example a prosthesis, artificial or otherwise-modified vessel, a valve, an intraocular lens, or a tissue implant. As used herein, the term “coat”, and related cognates such as “coated” and “coating,” refers to a process comprising of covering, in part or in whole, an inorganic structure with a composition described herein. For example and without limitation, coating of an inorganic structure with a disclosed composition, in the liquid phase, can include methods such as pouring, embedding, layering, dipping, spraying.

In another aspect, the disclosed composition is coated, in the liquid phase, onto a biocompatible structural material, such as a metal, an inorganic calcium compound such as calcium hydroxide, calcium phosphate or calcium carbonate, or a ceramic composition. Non-limiting examples of suitable metals are cobalt-chrome alloys, stainless steel alloys, titanium alloys, tantalum alloys, titanium-tantalum alloys, which can include both non-metallic and metallic components, such as molybdenum, tantalum, niobium, zirconium, iron, manganese, chromium, cobalt, nickel aluminum and lanthanum, including without limitation, CP Ti (commercially pure titanium) of various grades or Ti 6Al 4V (90% wt. Ti, 6% wt. Al and 4% wt. V), stainless steel 316, Nitinol (Nickel-titanium alloy), titanium alloys coated with hydroxyapatite. Metals are useful due to high strength, flexibility, and biocompatibility. Metals also can be formed into complex shapes and many can withstand corrosion in the biological environments, reduce wear, and not cause damage to tissues. Other compositions, including ceramics, calcium compounds, such as, without limitation, aragonite. Combinations of metal, ceramics and/or other materials also can be of use.

Any useful agent can be mixed into, co-delivered, co-applied or otherwise combined with any composition as described herein. For example, and without limitation, useful agents include interferons, interleukins, chemokines, monokines, hormones, coagulants, chemotherapeutics and antibiotics.

Antibiotics or antimicrobial agents may be added to the composition to reduce the potential for infection at the treatment site. A variety of antibiotics are known, including those that target the bacterial cell wall (for example, penicillins and cephalosporins) or the cell membrane (for example, polymixins), or interfere with essential bacterial enzymes (for example, quinolones and sulfonamides). Antibiotics include, but are not limited to, clindamycin, erythromycin, tetracycline, minocycline, doxycycline, penicillin, ampicillin, carbenicillin, methicillin, cephalosporins, vancomycin, and bacitracin, streptomycin, gentamycin, chloramphenicol, fusidic acid, ciprofloxacin and other quinolones, sulfonamides, trimethoprim, dapsone, isoniazid, teicoplanin, avoparcin, synercid, virginiamycin, cefotaxime, ceftriaxone, piperacillin, ticarcillin, cefepime, cefpirome, rifampicin, pyrazinamide, ciprofloxacin, levofloxacin, enrofloxacin, amikacin, netilmicin, imipenem, meropenem, linezolid, pharmaceutically acceptable salts thereof, and prodrugs thereof. Antibacterial agents also include cyclic lipopeptides (such as daptomycin), glycylcyclines (such as tigecycline), and oxazolidinones (such as linezolid). The antibiotic can be a narrow-spectrum or broad-spectrum antibiotic. The antibiotic can target gram negative or gram positive bacteria. Topical antibiotic can be included, such as a macrolide antibiotic (such as erythromycin), a sulfa antibiotic (such as sulfacetamide), a cyclic peptide (such as bacitracin a polymyxin) a pseudomonic acid (such as mupirocin), an aminoglycoside (such as neomycin), or a quinolone (such as ciprofloxacin or ofloxacin), a nitroimidazole (such as metronidazole), or a combination of drugs (such as bacitracin/polymyxin or neomycin/polymyxin B/bacitracin).

Additionally, local anesthetics may be added to the composition to minimize discomfort, such a lidocaine. Any appropriate additive may be utilized as long as it is compatible with the composition and the particular patient and disease state being treated.

In some aspects, the composition, such as the sterilized composition, is injectable through a 5Fr/16G catheter. in one aspect, the composition is injectable through a 5 Fr/16G catheter at room temperature, or at both room temperature and about 37° C.

### Methods of Use

Macrophages have been shown to be important regulators of normal healing following injury, and in normal tissue development. The disclosed compositions can recapitulate the effects of whole ECM on macrophage phenotype, leading to an increase in M2-like, regulatory, or pro-remodeling macrophages. Thus, any of the compositions disclosed herein can be used for modifying macrophage phenotype, such as for inducing regulatory M2 macrophages. For example, ECM hydrogels, whether acoustic ECM hydrogels or enzymatic ECM hydrogels, can be combined with exogenous MBV to provide compositions for modifying macrophage phenotypes.

In some aspects, methods are disclosed for inducing M2 macrophages in a subject by administering a therapeutically effective amount of a composition as disclosed herein, thereby inducing M2 macrophages in the subject. In further aspects, methods are disclosed for decreasing M1 (proinflammatory) macrophages in a subject. The methods include administering a therapeutically effective amount of a disclosed composition, thereby inhibiting the M1 macrophages in the subject. The subject can be any subject of interest, including human and veterinary subjects.

The disclosed compositions increase hemostasis at a lesion in a subject. Thus, methods are also disclosed for accelerating clotting and/or decreasing bleeding time of a wound. In some aspects, hemostasis is induced within about 10 to about 100 seconds after administering the acoustic ECM hydrogel containing exogenous MBV to the subject, such as about 10, 20, 30, 40, 50, 60, 70, 80, 90 or 100 seconds. The disclosed compositions are of use for treating stroke.

In some aspects, a therapeutically effective amount of the composition can be locally administered to a site in a subject to induce hemostasis. The subject can have a wound. The wound can be an external wound, or in internal wound not viable from outside the patient. The disclosed compositions are of use as a hemostatic agent at any type of wound. The method can include selecting any one of the subjects of interest, such as those with any wound.

In some aspects, methods are disclosed for treating a subject with inflammation or a wound. The method includes locally applying a therapeutically effective amount of the composition to the inflammation or the wound. In some aspects, the inflammation or wound is in the stomach, such as an ulcer. In some aspects, the inflammation or wound is in the throat, e.g., such as an ulcer. The compositions disclosed herein can be applied to the area of inflammation or wound or inflammation in the stomach or ulcer by local administration, such as topical administration, injection, or by injestion or through modes of enteral administration. In some non-limiting examples, the subject has an inflammatory disorder, such as, but not limited to, inflammatory bowel disease, ulcerative colitis, Crohn's disease, or rheumatoid arthritis. The method can include applying the ECM hydrogel to a tissue surface.

For example, methods are disclosed for treating inflammatory bowel disease, such as ulcerative colitis or Crohn's disease in a subject by administering to a subject an effective amount of an ECM hydrogel composition containing exogenous MBV as disclosed herein. The composition may be administered to subjects, e.g., human subjects, for example, by enema, by oral ingestion, by local administration into the bowel, e.g., local injection, or by systemic administration, e.g., by intravenous administration. For example, administration may be enteral, e.g., by mouth. For example, the administration may be directly to the bowel by enema. The compositions are administered in an amount sufficient to reduce inflammation in the bowel associated with inflammatory bowel disease, such as ulcerative colitis, or Crohn's disease. For example, the compositions are administered in an amount to reduce inflammation in the bowel as compared to the level of inflammation prior to administration of the composition. The compositions may be administered to the subject every week, every other week, monthly, every two months, or every three months, for example. The methods may also reduce symptoms associated with inflammatory bowel disease.

The disclosed compositions and methods can be used to treat Crohn's disease. A variety of types of Crohn's disease can be treated using the disclosed methods and compositions, including ilcocolic Crohn's, Crohn' colitis, Gastroduodenal Crohn's, and Jejunoileitis. Crohn's disease can be treated that is caused by an agent, such as Crohn's disease caused by immune system dysfunction (for example, autoimmunity or impaired innate immunity), genetic factors, changes in gut bacteria, and environmental factors. A variety of techniques can be used to identify a subject with Crohn's disease. For example, testing for Crohn's disease can include endoscopy (such as a colonoscopy), imaging (such as using a barium follow-through X-ray, CT scans, and MRI scans), and blood tests (such as to identify an iron, a vitamin D, or a vitamin B12 deficiency; erythrocyte sedimentation rate (ESR); and C-reactive protein levels). The methods of administering the compositions disclosed herein can decrease the severity or frequency of flare-ups of Crohn's disease. The methods can also lead to clinical remission and endoscopic remission of Crohn's disease. For example, in some aspects, administration of the disclosed compositions results in a decrease in the Crohn's Disease Activity Index (CDAI) as compared to the score prior to treatment. In one aspect, a patient experiences a reduction in CDAI score to below 150 and experiences remission. In another aspect, a patient experiences a reduction in CDAI score to below 450 or less (450 or greater is indicative of severe disease). In another aspect, a patient experiences a fall of at least 70 CDAI points (indicative of therapeutic response) as a result of receiving treatment according to the methods disclosed. For example, in another aspect, a patient experiences a fall of at least 70 CDAI points (indicative of therapeutic response) as a result of treatment from the time of administration of the disclosed compositions that remains decreased over 1 month, 2 months, or 3 months or more from administration. In one aspect, the subject experiences a decrease in the CDAI score from the time of administration of the disclosed compositions that remains decreased over 1 month, 2 months, or 3 months from administration. In one aspect, the subject's CDAI score decreases within 1 month, 2 months, or 3 months of being treated with a disclosed composition. In one aspect, the therapeutic effect in treating Crohn's disease extends beyond the duration of the treatment course, for example, by one month, two months, three months, four months, five months, six months or more.

The disclosed compositions can be used to treat a subject with ulcerative colitis. A variety of techniques can be used to identify a subject ulcerative colitis. For example, testing for ulcerative colitis can include a complete blood count (such as to identify anemia or thrombocytosis), electrolyte or kidney function tests (such as to identify hypokalemia, hypomagnesemia, or pre-renal failure, liver function tests (such as to identify primary sclerosing cholangitis), X-ray, urinalysis, stool culture (such as to identify parasites or infectious agents), erythrocyte sedimentation rate or C-reactive protein measurement (such as to identify inflammation), or sigmoidoscopy (such as to identify ulcers in the large intestine. In some examples, the clinical colitis activity index can be used to assess the severity of the ulcerative colitis. In some aspects, the disclosed compositions are administered by oral administration. The disclosed compositions can be administered locally, such as to the gut or bowel, e.g. enterally or by enema or injection. The methods can decrease the severity or frequency of flare-ups of ulcerative colitis. The methods can also lead to clinical remission and endoscopic remission of ulcerative colitis disease. For example, in some aspects, administration of the disclosed compositions results in a decrease in the Mayo Score or Ulcerative Colitis Disease Activity Index (UCDAI) for ulcerative colitis as compared to the score prior to treatment. In one aspect, a patient experiences a reduction in Mayo score to 2 or less and experiences remission. In another aspect, a patient experiences a reduction in Mayo score to 5 or less. In another aspect, a patient experiences a reduction in Mayo score to less than 10. In one aspect, the subject experiences a decrease in the Mayo score or UCDAI score within a certain time period after receiving treatment with the disclosed compositions, e.g., within 1 month, 2 months, or 3 months from administration. In one aspect, the therapeutic effect in treating ulcerative colitis extends beyond the duration of the treatment course, for example, by one month, two months, three months, four months, five months, six months or more from the time of receiving the treatment.

The disclosed compositions can be effective in increasing the number, ratio, or proportion of M2 macrophages in a subject's gastrointestinal tract as compared to M1 macrophages, in order to treat inflammatory bowel disease, such as Crohn's or ulcerative colitis. For example, the number, proportion, or ratio of M2 macrophages to M1 macrophages increases in the subject's gastrointestinal tract, e.g., the colon, upon a subject being treated with the disclosed compositions. This increase can occur within at least one week, two weeks, or one month, for example, from administration of the compositions disclosed herein.

As a result, for example, histopathological evaluation of the subject's gastrointestinal tissues, e.g., colon tissue, will exhibit fewer phenotypic features characteristic of inflammation and disease (e.g., disrupted mucosa, absence of confluent layer of epithelial cells, increased cellularity in the lamina propria and submucosa) and more phenotypic features characteristic of healthy bowel tissue, e.g., colon tissue (e.g., intact muscularis mucosa, presence of healthy confluent layer mucosal epithelial cells, and normal cellularity in the lamina propria and submucosa) upon treatment with compositions and methods disclosed herein. In other non-limiting examples, the subject is an organ transplant recipient, a subject with graft versus host disease, a subject with myocardial infarction, or a subject with a wound, such as, but not limited to, a subject with a surgical wound or a non-surgical traumatic wound. Thus, disclosed in a method for accelerating wound healing and/or increasing hemostasis in an individual in need thereof, comprising administering a therapeutically effective amount of a composition as disclosed herein. The administration can be local, to the site of the wound or graft.

The composition can be applied to any wound site to increase hemostasis and/or increase wound healing. The wound can be a wound in the skin, or a wound on any surface, including, but not limited to, the eye. Methods are also provided for wounds that result from ischemia and ischemic injury, such as chronic venous leg ulcers caused by an impairment of venous circulatory system return and/or insufficiency. Thus, the present methods can utilize topical dermal or ocular administration.

Generally, in these applications, the composition is formulated for topical administration. The hydrogels can be applied to a tissue surface of any organ. For example, the compositions disclosed herein can be applied to the esophagus to treat esophagitis. Upon administration of the disclosed compositions to a subject suffering from esophagitis, e.g., administration to the esophagus, inflammation in the esophagus can be reduced. Further, the disclosed compositions can be effective in increasing the number, ratio, or proportion of M2 macrophages in a subject's esophagus as compared to M1 macrophages, in order to treat esophagitis. For example, the number, proportion, or ratio of M2 macrophages to M1 macrophages increases in the subject's esophageal tissue upon a subject being treated with the disclosed compositions. This increase can occur within at least one week, two weeks, or one month, for example, from administration of the compositions disclosed herein.

Topical compositions to heal wounds, such as dermal wounds, are disclosed herein. These wounds amenable to treatment may be of superficial nature or may be deep and involve damage of the dermis and the epidermis of skin. The wound can be a surgical wound. Thus, methods are provided to promote wound healing in a subject, and/or promote clotting (increase hemostasis) in the subject.

The composition can be applied directly to the target location, for example in a topical preparation such as a sheet, plug, or as a part of a dressing or a bandage. Bandage and wound dressings may contain the composition. These may be prepared by applying the composition, together with any other additives desired, to a bandage or wound dressing. These, sheets, plugs, bandages or dressings can be used to decrease clotting time and or to increase wound healing. The acoustic hydrogel can be administered by injection to the target location to promote wound healing, for example, as a solid in the gel phase, or the temperature can be raised above 37° C. prior to administration such that the hydrogel is administered in the liquid phase.

The composition can be applied a single time. Alternatively, the acoustic ECM hydrogel can be applied to the affected area periodically, typically from about 1 to 10 times each day, such as, for example, over a period of from about 3 to 14 days, depending on the nature of the wound. In some cases, it may be desirable to apply the compositions indefinitely. For example, ECM hydrogels, whether acoustic ECM hydrogels or enzymatic ECM hydrogels, combined with exogenous MBV can be applied locally, e.g., topically, to a tissue of the body in the subject, whether that tissue is external, like the skin, or internal, such as the colon, small intestine, esophagus, throat or stomach.

In some aspects, the composition increases hemostasis and/or wound healing at least 20%, at least 30%, at least 40%, at least 50%, at least 60%, at least 70%, at least 80%, at least 90%, at least 95%, at least 100%, or at least 200%, as compared to a control, such as a standard value, the rate of wound healing or hemostasis achieved without treatment, or with treatment of the ECM hydrogel alone or the MBV alone.

The disclosed compositions can also be used in the treatment of a surgical wound and other intentional interventions where the compositions may be applied immediately after completion of the surgery. Methods are provided for stimulating healing of wounds, and increasing hemostasis at a wound site, including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, and burns resulting from heat exposure or chemicals.

The subject can be any mammalian subject of interest, including a human or a veterinary subject. The subject can be a child or an adult subject, such as a young, middle aged, or older adult subject. In humans, an adult subject is greater than 18 years of age, a young adult is about 18 to about 35 years of age, a middle aged adult is generally considered to be about 35 to about 55 years of age, and an elderly (or aged) human subject is more than about 55 years old, such as more than 60 years old, more than 65 years old, more than 70 years old, more than 75 years old or more than 80 years old.

The subject can heal wounds at a normal rate or can be healing impaired. A number of afflictions and conditions can result in healing impairment. These include diabetes (such as Type II diabetes mellitus), treatment with both steroids and other pharmacological agents, and ischemic blockage or injury (as in peripheral vascular disease or traumatic vascular occlusion). Conditions which induce abnormal wound healing, include, but are not limited to uremia, malnutrition, vitamin deficiencies, obesity, infection, immunosuppression and complications associated with systemic treatment with steroids, radiation therapy, and antineoplastic drugs and antimetabolites. Steroids which have been shown to impair wound healing include cortisone, hydrocortisone, dexamethasone, and methylprednisolone. Non-steroid compounds, such as octreotide acetate, have also been shown to impair wound healing (Waddell et al., Am. Surg. 63:446 449, 1997).

The subject can have a clotting disorder, or can be undergoing treatment with anticoagulants, such as, but not limited to warfarin or PLAAVIX®. The subject can have a Factor II, V, VII, X, or XII deficiency. The subject can have hemophilia A, hemophilia B, von Willebrand's disease, a deficiency or structural abnormalities in fibrinogen, or prothrombin. Thus, in some aspects, these subjects are selected for treatment.

Methods are also provided herein to increase the adherence of skin grafts to a wound bed and to stimulate re-epithelialization from the wound bed. Types of grafts include, but are not limited to: autologous skin graft, artificial skin, allografts, autodermic graft, autoepidermic grafts, avascular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omental graft, patch graft, pedicle graft, penetrating graft, split skin graft, thick split graft. The methods include administering to the subject with the graft a therapeutically effective amount of the compositions disclosed herein, thereby increasing the adherence and acceptance of the graft and controlling or eliminating bacterial growth. In some aspects, cells or a tissue treated with the composition are transplanted into a subject. In one specific, non-limiting example, the composition is administered to a graft, such as a skin graft, prior to transplantation.

Methods are also provided to treat blisters and burns due to abrasion or chemical injury. These methods include the treatment of the skin or internal organs. These methods include treatment of ovary injury, for example, due to treatment with chemotherapeutics or treatment with cyclophosphamide; radiation- or chemotherapy-induced cystitis; or high-dose chemotherapy-induced intestinal injury. The methods include administering to the subject a therapeutically effective amount of a composition as disclosed herein to promote healing of the blisters or burns and to reduce or eliminate bacterial growth.

Methods are provided for promoting the healing of anastomotic and other wounds caused by surgical procedures in individuals. These methods include administration of an effective amount of the compositions disclosed herein, after, and/or during anastomotic or other surgery. Anastomosis is the connecting of two tubular structures, for example, when a mid-section of intestine is removed and the remaining portions are linked together to reconstitute the intestinal tract. Unlike cutaneous healing, the healing process of anastomotic wounds is generally obscured from view. Further, wound healing, at least in the gastrointestinal tract, occurs rapidly in the absence of complications; however, complications often require correction by additional surgery (Thornton and Barbul, Surg. Clin. North Am. 77:549 573 (1997)). The method can include selecting a subject in need of anastomotic wound healing. The subject can be a subject with impaired wound healing due to one of the conditions above, or can be a subject that has normal wound healing, such as a subject that does not have any of the conditions listed above.

In some aspects, the compositions are used to treat or prevent surgical adhesions, e.g., the formation of bands of scar tissue that bind tissues or organs that would not normally be connected. Compositions disclosed herein comprising an ECM hydrogel containing exogenous MBV can be placed on the surface of tissues or between tissues where an adhesion is not desired, e.g., by topical application during a surgical procedure, or injection. The surgical procedure may be an abdominal surgical procedure such as hernia procedure, a caesarean section, an appendectomy, or resection of any portion of the gastrointestinal tract such as the colon, rectum or anus. For example, the disclosed composition is applied to the abdominal wall and the tissue or organ that is the cause of the hernia, e.g., the small intestine, large intestine, or stomach. The disclosed composition is applied to the uterus and surrounding tissue to prevent adhesion after caesarean section.

In some aspects, the compositions are subject to sterilization. Sterilization is important to ensure compositions are sufficiently removed of contamination from pathogens and suitable for medical use, such as implantation into a human or animal body. Methods such as gamma irradiation, ethylene oxide, supercritical CO₂, hydrogen peroxide gas plasma, or ozone may be suitable for sterilization, although other methods of sterilization known in the art may also be suitable. Gamma sterilization is an acceptable method of sterilization.

### Use as a Submucosal Cushion

Endoscopy is a procedure that allows examination of the interior of a hollow organ or cavity of the body by means of an instrument called an endoscope, without employing invasive surgery. Endoscopy can be used for surgical procedures such as cauterization of a bleeding vessel, removing polyps, adenomas and small tumors, performing biopsies or removing a foreign object. Endoscopic procedures can be performed in the gastrointestinal tract, the respiratory tract, the ear, the urinary tract, the female reproductive system and, through small incisions, in normally closed body cavities such as the abdominal or pelvic cavity (laparoscopy), the interior of a joint (arthroscopy) and organs of the chest (thoracoscopy and mediastinoscopy). Endoscopy can be performed in the upper gastrointestinal tract or the lower gastrointestinal tract. The endoscope is an illuminated, usually fiber optic, flexible or rigid tubular instrument for visualizing the interior of a hollow organ or part (such as the bladder, esophagus, stomach or intestine) for diagnostic or therapeutic purposes, that typically has one or more working channels to enable passage of instruments (such as forceps, electrosurgical knife, endoscopic injection needles or scissors) or to facilitate the removal of bioptic samples. It includes a suitable lamp and imaging device at its distal portion, and it can be inserted through natural occurring openings of the body, such as the mouth, the anus, the ear, the nose or through small surgical incisions. Given the wide variety of body organs or cavities which can be examined by means of endoscopic procedures, several types of specialized endoscopes exist, such as, for example, laryngoscope, thoracoscope, angioscope, colonoscope, enteroscope, sigmoidoscope, rectoscope, proctoscope, anoscope, arthroscope, rhinoscope, laparoscope, hysteroscope, encephaloscope, nephroscope, esophagoscope, bronchoscope, gastroscope, amnioscope, cystoscope.

Endoscopic procedures are widely applied in the gastrointestinal tract, including the upper and the lower gastrointestinal tract. For example, endoscopic procedures can be used to examine the mucosa that covers the gastrointestinal cavities, and to detect small and large pathological lesions, such as inflammatory tissue, polyps, pseudo-polyps, serrated lesions, adenomas, ulcerations, dysplasias, pre-neoplastic and neoplastic formations, and tumors. Endoscopic procedures can be used for biopsies and removal of pathologic lesions (polyps, adenomas, dysplasias, pre-neoplastic and neoplastic formations, tumors). Surgical interventions include two types of endoscopic resection procedures commonly used in gastrointestinal endoscopy to remove pathological lesions: endoscopic mucosal resection (EMR) and endoscopic submucosal dissection (ESD). These two techniques allow for minimally invasive treatment of gastrointestinal polyps, adenomas, dysplasias, and early-stage cancers that involve a minimum risk of lymph-node metastasis.

Methods are disclosed herein for dissecting a mucosa and a submucosa from a muscularis propria from a region of an organ of a subject. The organ can be in the gastrointestinal tract, for example, the esophagus, the duodenum, stomach, small intestine, large intestine (colon) or rectum. The organ can be the bladder, organs of the oral-respiratory system (lungs, throat (pharynx), tongue, nasal passages, sinuses), the skin, or the uterus and vaginal tract. Examples of specific tissues are respiratory epithelium, nasal epithelium, dermal or epidermal tissue and uterine epithelium. One exemplary organ is the esophagus. Another exemplary organ is the colon. The methods are of use in any organ that has a mucosa and a submucosa, wherein a superficial lesion can be formed, such as a malignant or pre-malignant lesion.

These methods include injecting submucosally into the organ of the subject the disclosed composition of an ECM hydrogel containing exogenous MBV to form a cushion between the submucosa and the underlying muscularis propria at the region of the organ. In one aspect, the organ is not the esophagus. In another aspect, the organ is the esophagus. In another aspect, the organ the large intestine (colon). The method can be an endoscopic mucosal resection (EMR) or an endoscopic submucosal dissection (ESD).

EMR is an endoscopic technique developed for removal of sessile or flat neoplasms confined to the superficial layers (mucosa and submucosa) of the gastrointestinal (GI) tract. EMR is typically used for removal of lesions smaller than 2 cm or piecemeal removal of larger lesions. EMR also plays an important role in the assessment of resected specimens for accurate pathological staging. In contrast to polypectomy, EMR involves the lifting up of a lesion from the muscular layer by injecting a fluid agent, commonly normal saline (NS) solution, into the submucosal layer. EMR is also useful for obtaining specimens for accurate histopathological staging to determine the risk of lymph-node metastasis. EMR facilitates the complete removal of the affected mucosa by excising through the middle or deeper portion of the gut wall submucosa. Various EMR techniques have been described and four methods involving snare resection are commonly used: (1) the inject and cut method; (2) the inject, lift, and cut method; (3) cap-assisted EMR (EMRC); and (4) EMR with ligation (EMRL). In the inject and cut technique, the diseased mucosa is lifted up from the muscular layer by creating a submucosal fluid cushion of an ECM hydrogel composition containing exogenous MBV, captured, strangulated using an electrosurgical snare, and then resected. However, injection into the thin submucosal layer is a delicate process, the injected solution tends to dissipate quickly, flat and depressed lesions are hard to capture with the snare compared with protruded lesions, and large or awkwardly located lesions can be difficult to remove (Uraoka et al., Drug Design, Development and Therapy 2008:2 131-138). Injection-assisted EMR is frequently used for large flat colon polyps.

Endoscopic submucosal dissection (ESD) was specifically developed for removing larger lesions. Lesions are dissected directly along the submucosal layer using an electrosurgical knife, resulting in an en-bloc resection of even large lesions. ESD has been predicted to replace conventional surgery in treating certain cancerous stages, but since it has a higher rate of perforation and bleeding complications than conventional EMR, a greater degree of endoscopic skill and experience is required than for EMR. ESD can use numerous electrosurgical knives, such as an insulation-tipped diathermic knife, a needle knife, a hook knife, a flex knife, a triangle tipped knife, a flush knife, splash needle, and a small-caliber tip transparent hood. These knives can be used with a high frequency electrosurgical current (HFEC) generator. ESD is characterized by three steps: (1) injecting a material such as an ECM hydrogel composition containing exogenous MBV to form a submucosal cushion to elevate the lesion from the muscle layer; (2) circumferential cutting of the surrounding mucosa of the lesion; and (3) dissection of the connective tissue of the submucosa beneath the lesion (see Kakushima et al., Wold J. Gstroenterol. 14 (9): 2962-2967, 2008, incorporated herein by reference. Various submucosal injection solutions had previously been developed and shown to be satisfactory for use during EMR, but introduction of the lengthier ESD procedure required a longer-lasting solution to help identifying the cutting line during dissection of the submucosal layer (Uraoka et al., Drug Design, Development and Therapy 2008:2 131-138). The presently disclosed methods meet this need.

A submucosal injection is used in EMR, as injection of fluid into the submucosa cushions facilitates the isolation of the tissue to be removed just before capture of the target lesion, such as with a snare, thereby reducing thermal injury and the risk of perforation and hemorrhage while also facilitating resection. Submucosal injection plays an important role in the EMR procedure, as the solution must be retained in place for sufficient duration and needs to form a hemispheric shape to facilitate snaring. In addition, providing a sufficiently high submucosal elevation results in safe submucosal cutting during the ESD procedure (Uraoka et al., Drug Design, Development and Therapy 2008:2 131-138). Furthermore, as inflammation results from the procedure, any cushion retained at the procedure site should have anti-inflammatory properties. The disclosed compositions of ECM hydrogel including exogenous MBV will mitigate stricture and promote re-epithelialization. The presently disclosed methods also meet this need.

In some aspects, the disclosed composition has anti-inflammatory properties, and is inexpensive, non-toxic, easy to inject and provides a high, long-lasting submucosal cushion. The composition is administered in its gel state at the site of injection to form a cushion. The cushion can be dissected during the procedure so that some hydrogel remains on the underlying muscularis propria, thereby aiding healing. The disclosed composition facilitates closure of the wound created by removal of the resected mucosa/submucosa. In some aspects, the procedure is an ESD. In other aspects, the procedure is an EMR.

Normal saline solution (NS) and thinner solutions (e.g., ELEVIEW™, see U.S. Pat. No. 9,226,996, incorporated herein by reference) have been used as submucosal cushions for endoscopic resection, but the inherent characteristics of these solutions make it difficult to produce the proper submucosal fluid cushion, maintain the desired height, and retain the cushion at the desired location, because of the rapid dispersion of the solution. Furthermore, in ESD, once the mucosa/submucosa are removed, these agents will not be retained on the underlying muscularis propria. Furthermore, these agents to not aid the healing process, such as by reducing inflammation. The use of a disclosed composition including an ECM hydrogel containing exogenous MBV meets these needs.

A composition, such as an ECM hydrogel incorporating MBVs, as disclosed herein can be used as in any ESD or ESR. As disclosed in U.S. Pat. No. 9,364,580, incorporated herein by reference, endoscopic injection needles are devices which can be long (up to about 230) cm and which include a relatively long catheter within which an inner injection tube having a distal injection needle is slideably disposed. A proximal actuating handle is coupled to the catheter and the injection tube for moving one relative to the other when necessary. Fluid access to the injection tube is typically provided via a leer connector on the handle. Endoscopic injection needle devices are typically delivered to the injection site through the working channel of the endoscope. In order to protect the lumen of the endoscope working channel from damage, the handle of the infusion needle device is manipulated to withdraw the distal injection needle into the lumen of the catheter before inserting the device into the endoscope. This prevents exposure of the sharp point of the injection needle as the device is moved through the lumen of the endoscope. When the distal end of the endoscopic injection needle device is located at the injection site, its handle is again manipulated to move the injection needle distally out of the lumen of the catheter. When advanced to the most distal position, the exposed portion of the injection needle is approximately 4-6 mm in length.

After the injection site has been pierced, the composition including the ECM hydrogel (e.g., an enzymatically or acoustically produced ECM hydrogel) and MBVs, usually contained in a 5 ml to 10 ml syringe provided with a luer-lock fitting connected to the handle of the injection needle, can be delivered through the injection tube and the needle into the injection site, such as between the submucosa and the underlying muscularis propria.

The injection needle and other accessories commonly used during endoscopic procedures, such as snares for polypectomy, clipping devices, biopsy forceps and similar, are passed through one or more specific channels of the endoscope, usually called working channels or operating channels. Depending upon the type of endoscope used in GI endoscopy (e.g. gastroscope, enteroscope, colonoscope, duodenoscope, sigmoidoscope and similar), the inner diameter of the working channels may vary considerably. However, the most common endoscopes used in GI endoscopy have working channels with inner diameter in the range from about 2 mm to about 5 mm. Generally, the manufacturers of endoscopic accessories produce accessories having outer diameters which allow them to fit all the working channels. In some aspects, the endoscopic injection needles, the outer diameter of catheter ranges from 1.9 mm to 2.3 mm, such as about 1.9, 2.0, 2.1, 2.2 or 2.3 cm. Thus, considering that the inner injection tube is contained in the outer catheter, its internal diameter is usually 1 mm or less. The disclosed ECM hydrogel in gel or liquid form, can readily pass through these catheters.

The composition including the ECM hydrogel and exogenous MBVs can be used in an endoscopic resection procedure by sucking a volume of the hydrogel from its primary container by means of a syringe, injecting a suitable volume of said hydrogel by means of an endoscopic injection needle inserted in the working channel of the endoscope immediately under the superficial mucosal layer, to depose the composition into the submucosal layer that becomes a cushion when in place: the elevation of the mucosal surface allow the endoscopist to perform an easy resection of the mucosal lesion found during the execution of the endoscopic procedure even if the lesion is flat and thus not protruding into a lumen, such as an intestinal, esophageal, or gastric lumen. At body temperature, an acoustic ECM hydrogel is a viscous yet flowable gel transitioning to the liquid phase and can be easily injected under the superficial mucosal layer to form a cushion for this procedure. Because the gel-sol transition takes time, the cushion remains in place for a sufficient time for the resection to take place. Enzymatic ECM hydrogels containing exogenous MBV are also suitable for such endoscopic procedures as they begin to solidify at body temperature to provide sufficient support for resection of tissue and remain in place to promote healing of the resection site.

The presence of at least one dye into the cushion can aid an endoscopist to visualize the structures beneath the mucosa (e.g. the submucosal layer and the external muscular wall), thereby lowering the risk that the endoscopist, performing the resection procedure, may cause damages to said structures. The use of the dye can allow visualization of the cushion cavity and the mucosal basement. The removal of the lesion from the mucosal surface generates a mucosal wound. The persistence of the cushion generated by the injected volume of the pharmaceutical composition allows the endoscopic resection procedure to be performed without the need to re-inject. The acoustic ECM hydrogel is injected submucosally into a region of interest in the organ of the subject, such as at the region of a lesion or tumor, to form a cushion between the submucosa and the underlying muscularis propria at the region of the organ. The cushion can be dissected, such that a portion of the composition is maintained on the underlying muscularis propria and aid in the healing process.

The disclosed methods are of use, for example, in the esophagus. In a non-limiting example, the method comprises a method of dissecting an esophageal carcinoma or adenocarcinoma from the esophagus. In another non-limiting example, the method comprises dissecting the mucosa and the submucosa from the esophagus of a subject who has Barrett's esophagus. In these aspects, the ECM hydrogel can be made from urinary bladder, a small intestinal submucosal (SIS), an esophageal, a trachea, a liver or a dermal ECM.

The disclosed methods are also of use in other organs. The organ can be any organ of interest, such as an organ of the gastrointestinal tract. The organ may be in the upper gastrointestinal tract such as the pharynx, tongue or mouth. The organ may be the bladder, vaginal tract, or uterus. In some aspects, the organ is the colon, duodenum, stomach, cecum, colon, sigmoid colon, rectum, small intestine or large intestine. In one non-limiting example, the organ is the stomach, the small intestine or the large intestine, and the method comprises a method of dissecting a carcinoma or adenocarcinoma from the stomach. In a further non-limiting example, the organ is the colon, and wherein the method comprises dissecting a polyp or a carcinoma from the colon. In these aspects, composition can include an acoustic ECM hydrogel can be a urinary bladder, a small intestinal submucosal, an esophageal, a trachea, a liver or a dermal acoustic ECM hydrogel. The MBV can be from the same or a different source.

The composition including the ECM hydrogel and exogenous MBV, as disclosed herein, is maintained at a temperature at or below which it gels for application as a submucosal cushion.

The composition including the ECM hydrogel (e.g., acoustic hydrogel) and exogenous MBV can be maintained, for example, at about 4° C. or at about room temperature prior to administration. In one aspect, the acoustic ECM hydrogel can be administered at a temperature, for example, from 4° C. to below 37° C., or from 4° C. to 25° C. In one aspect, the composition is administered at a temperature below 37° C. An effective amount of the composition as a gel is then utilized. The composition, including the acoustic ECM hydrogel and exogenous MBVs remains as a gel in the tissue of the subject, which is at a temperature of approximately 37° C. In one aspect, the gel to sol transition of the acoustic ECM hydrogel, with the exogenous MBVs, is at about 37° C., such that the composition can be used as a submucosal cushion because it is sufficiently viscous at body temperature.

In some aspects, the ECM concentration in the hydrogel is 25 mg/ml to about 200 mg/ml, such as about 25 mg/ml to about 100 mg/ml. In other aspects, the ECM concentration in the hydrogel is about 50 to about 150 mg/ml, such as about 75 to about 125 mg/ml, such as about 75, 80, 85, 90, 95, 100, 105, 110, 115, 120 or 125 mg/ml. In a specific non-limiting example, the ECM concentration in the hydrogel is about 100 mg/ml.

The compositions can be provided in a lyophilized form at either room temperature, a cold temperature (for example, about 4° C.) or frozen (for example, at about −20° C.), and reconstituted just prior to administration to the anatomic region of interest in the subject.

The disclosed methods are of use in any subject, including human and veterinary subjects. The subject can be any age. The subject can be an adult or a juvenile. In one aspect, a composition including an ECM hydrogel is injected in a target tissue in an organ to form a cushion which is then optionally subjected to an endoscopic surgical procedure, such as a resection procedure. The ECM can be from the same species as the subject being treated, or can be from a different species. In some aspects, the subject is human, and the ECM hydrogel and/or MBVs are derived from human or porcine ECM. In other aspects, the ECM hydrogel and/or MBVs are derived from a non-human primates, dog, cat, horse, or cow. The ECM can also be from a commercial source. In some aspects, the ECM hydrogel and MBV can, in some aspects, be derived from any mammalian tissue, such as but not limited to porcine or human tissue, and, in some non-limiting examples, are from the urinary bladder, small intestine, or the esophagus.

The disclosed methods are invasive, as they require an injection that dissects a mucosa and a submucosa from a muscularis propria from a region of an organ of an intestinal tract of a subject. In some aspects, the composition is not applied to a surface of an organ, such as an organ of the gastrointestinal tract, such as the esophagus. The disclosed methods can be used in the esophagus, but can also be used in other tissues. In other aspects, the composition is applied to the surface of the organ.

Any of the methods disclosed herein can include injecting submucosally into the organ of the subject a pharmaceutical composition including an ECM hydrogel and exogenous MBVs to form a cushion between the submucosa and the underlying muscularis propria at the region of the organ. The composition gels and dissects the mucosa and the submucosa from the underlying muscularis propria and inhibits inflammation in the region of the organ in the subject. The composition as a gel can be administered endoscopically or via a catheter. In some aspects, the organ is the esophagus, colon, stomach, cecum, colon, sigmoid colon, rectum, small intestine or large intestine. The composition, as a gel or sol, also can be administered endoscopically or via a catheter.

In some aspects, the resection procedure is an endoscopic mucosal resection or an esophageal endoscopic submucosal dissection, and the method comprises a method of dissecting an esophageal carcinoma or adenocarcinoma from the esophagus. In more aspects, the method includes dissecting the mucosa and the submucosa from the esophagus of a patient who has dysplasia. In more aspects, the method includes dissecting the mucosa and the submucosa from the esophagus of a subject who has Barrett's esophagus.

In some aspects, the resection procedure is an endoscopic mucosal resection or an endoscopic submucosal dissection. In further aspects, the organ is the stomach, small intestine or large intestine, and the method comprises a method of dissecting a polyp, a carcinoma or an adenocarcinoma from the colon. In more aspects, the method includes dissecting the mucosa and the submucosa from an organ of a patient who has dysplasia. In specific non-limiting examples, the method comprises dissecting a polyp or a carcinoma from the colon.

The methods can also include performing an endoscopic resection procedure on the cushion. In some aspects, the methods include dividing the cushion such that hydrogel is retained on the underlying muscularis propria of the esophagus and the mucosa and the submucosa are removed from the region of the esophagus. In some non-limiting examples, the portion of the hydrogel cushion that is retained on the underlying muscularis propria downregulates pro-inflammatory macrophage activation in the esophagus.

The following examples are provided to illustrate particular features of certain aspects of the disclosure, but the scope of the claims should not be limited to those features exemplified.

### EXAMPLES

### Example 1: Treatment of Ulcerative Colitis

To document the effectiveness of the combined use of an ECM hydrogel and MBV, a study was performed to evaluate the healing of the colon following DSS-induced ulcerative colitis in rats.

### Overview of Study Design

The study population was 15 female Sprague-Dawley rats. Rats were randomly assigned to a healthy control group (N=3) or a dextran sodium sulfate (DSS)-treated group (N=12). Animals exposed to DSS were further divided into 5 subgroups: (1) no treatment control (N=3), (2) MBV Enema (N=2), ECM Hydrogel Enema (N=2), MBV-Infused Dermal ECM Acoustic Hydrogel Enema (N=2), and Multiple MBV Intravenous Injections (N=3).

TABLE 1
Experimental Groups and descriptions

| | Number of | |
|---|---|---|
| Treatment | Animals (N) | Description |
| Not DSS induced (−control) | 3 | Animals with healthy colons |
| No Treatment (+control) | 3 | Animals induced with 5.5% DSS and given no |
| | | treatment |
| MBV Enema | 2 | MBV + PBS given as an enema on days 0, 1, 2, 3 |
| ECM Hydrogel Enema | 2 | Dermal ECM Hydrogel given as an enema on |
| | | days 0, 1, 2, 3 |
| MBV Infused Hydrogel Enema | 2 | MBV infused dermal acoustic hydrogel given as |
| | | an enema on days 0, 1, 2, 3 |
| Multiple MBV IV Injection | 3 | MBV injection containing 3.3 × 10¹¹ particles given |
| | | intravenously on days 0 and 2 |

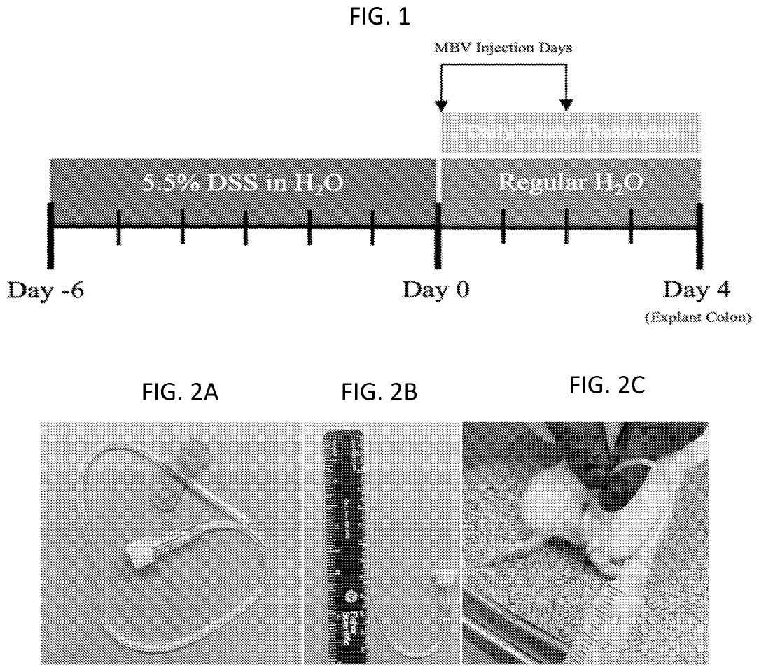

Ulcerative colitis (UC) was induced in rats by exposing rats with 5.5% dextran sodium sulfate (DSS) in drinking water and ad libitum drinking continued for 6 days leading up to treatment. Throughout disease induction, animals were assessed for weight loss, food and water consumption, stool consistency, and stool blood content. Treatments were given on the regimens shown in FIG. 1 and animals colons were explanted for histological analysis on day 4.

### Detailed Procedures

Animals and Husbandry: Animal studies were conducted in compliance with all regulations regarding the humane treatment of laboratory animals as set forth by the University of Pittsburgh's Institutional Animal Care and Use Committee. Female Sprague Dawley rats, 8-12 weeks of age, were obtained from a certified vendor (Jackson Labs). Following shipment, rats were environmentally acclimated for 7-10 days. Animals were housed in standard laboratory conditions with a temperature of 21-23° C. and 12-hour dark/light cycles. Rats were allowed ad libitum access to food and water. Upon study, animals were individually housed for appropriate data collection.

Dextran Sodium Sulfate Preparation and Administration: Dextran sodium sulfate (DSS) salt (36,000-50,000 molecular weight) was obtained from MP Biomedical. A 5.5% DSS solution was prepared in autoclaved tap water and administered by ad libitum drinking for 6 days.

MBV Isolation: Porcine urinary bladder extracellular matrix (ubECM) powder was obtained from ECM Therapeutics (Warrendale, PA). Enzymatic digestion of the UBM-ECM powder was performed (0.05 mg/mL Liberase, 5 mM CaCl₂) in 1×PBS) for 3 hours at 37° C. Enzymatically digested ECM was subjected to centrifugation at 10,000×g for 30 minutes at 4° C. Supernatant underwent ultracentrifugation at 28,000×g for 70 minutes. Pellets were washed once with PBS and then resuspended in particle-free 1×PBS. Average particle size and concentration of the MBV suspension determined through Nanoparticle Tracking Analysis (NTA) using the NanoSight NS500. MBV suspension (238 nm, 1.10×10¹² particles/mL) was stored at −20° C. until use.

MBV Enema: MBV suspension volume was increased to 5-mL in 1×PBS and placed in a 5 mL syringe resulting in a concentration of 1.38×10¹¹ particles per mL, or 6.9×10¹¹ particles per enema.

Dermal ECM Acoustic Hydrogel Enema: Dermal ECM powder was supplied by ECM Therapeutics (Warrendale, PA). A 50 mg/mL solution of dermal ECM powder in PBS was produced by sonicating the ECM for 5 minutes at 40% amplitude. The solution was then added to a 5-mL syringe and allowed set at 4° C. Syringes containing a 5-mL volume of Dermal ECM Acoustic Hydrogel were stored at 4° C. until use. MBV-Infused Dermal ECM Acoustic Hydrogel Enema: Dermal ECM powder was supplied by ECM

Therapeutics (Warrendale, PA). A 50 mg/mL solution of dermal ECM powder in PBS was produced and sonicated for 5 minutes at 40% amplitude. After 5-10 minutes of cooling at room temperature, 5 mL of MBV suspension was added to 35 mL of the sonicated solution and mixed gently resulting in an MBV concentration of 1.38×10¹¹ particles/mL. The solution was then added to a 5-mL syringe and allowed to gel resulting in 6.9×10¹¹ particles per enema. The syringes containing the hydrogel were stored at 4° C. until use.

MBV Intravenous Injection: A volume of 300 μL of MBV suspension in 1×PBS was placed in a 1-mL syringe containing 1.10×10¹² particles/mL, or 3.3×10¹¹ particles per injection.

Enema Delivery: Rats were anesthetized with inhaled 0.5-5% isoflurane and enemas were administered with a flexible SURFLO® winged infusion catheter (Terumo, OD=2 mm) (FIGS. 2A-C). The enema solutions were administered along the length of the colon utilizing a syringe attached to the catheter; specifically beginning at 8 cm all the way to the anus while gradually removing the catheter with an approximate total infusion time of 60 seconds. Syringes contained a total of 5 mL of designated treatment material. The rate of the enema delivery and volume of enema material was intended to ensure complete coverage of the colon from proximal to distal locations. Treated groups received daily enemas for 4 days beginning immediately upon the removal of 5.5% DSS from drinking water.

MBV Injection Delivery: Rats were anesthetized with inhaled 0.5-5% isoflurane and a 300 uL volume of material was administered into the lateral tail vein. Injections were given to appropriate groups immediately upon the removal of 5.5% DSS from drinking water and 2 days post DSS removal.

In-Life Observations: Trained personnel recorded the following data on a daily basis: (1) food consumption, (2) water consumption, (3) animal weight, (4) stool consistency, (5) stool blood presence. Stool was scored based on consistency (0=normal, 2=loose, 4=diarrhea) and presence of blood (0=none, 2=occult, 4=gross bleeding). Occult blood was tested using ColoScreen ES Lab Pack Fecal Occult Tests.

Colon Explant Protocol: Animals were sacrificed at day 4 as shown on FIG. 1. Euthanasia was achieved by CO₂ inhalation and subsequent cervical dislocation in accordance with the American Veterinary Medical Association (AVMA). Following euthanasia, the colon was resected via access through a midline incision. The colon spanning from the rectum to the cecum was incised longitudinally to expose the luminal surface and was rinsed with PBS prior to gross scoring of the tissue. The explanted colon was photographed and scored. The distal 8 cm of the colon was fixed in 10% Neutral Buffered Formalin for 2 days and submitted for paraffin embedding and tissue slicing (5 um). Samples were stained with Hematoxylin and Eosin in which representative images were taken and histologic examination and scoring were performed.

Gross Scoring of Colonic Tissue: Colons were assessed grossly for damage according to the metrics outlined in Table 2. Scores were determined by investigators that were blinded to the treatment group.

TABLE 2
Criteria for Gross Anatomic Scoring

| Score | Appearance |
|---|---|
| 0 | Normal |
| 1 | Localized hyperemia, no ulcers |
| 2 | Inflammation, no ulcers |
| 3 | Ulceration at one site and inflammation |
| 4 | Two or more sites of ulceration and inflammation |
| 5 | Ulceration at multiple sites extending >1 cm along |
| | the length of the colon |
| 6-10 | When an area of damage extended >2 cm along the colon, |
| | the score was increased by 1 for each cm |

Histologic Evaluation: The explanted tissues were processed for microscopic analysis as described previously. The H&E-stained sections were scored for morphologic changes in the colonic tissue as described in Table 3. A separate score was given for the extent of ulceration, mild mucosal inflammation, severe mucosal inflammation, mild submucosal inflammation, and severe submucosal inflammation for each tissue section. Scores were determined by investigators that were blinded to treatment group. A guidance document that defined scoring criteria and showed examples was provided to each scorer for uniformity of scoring.

TABLE 3
Criteria for Histologic Scoring of H&E-Stained Sections

| | | |
|---|---|---|
| Criteria | Score | Description |

| | | | |
|---|---|---|---|
| Ulceration | Present? | 0 | None |
| Mucosal | Mild | 1 | 1-10% |
| Inflammation | Severe | 2 | 11-30% |
| Submucosal | Mild | 3 | 31-60% |
| Inflammation | Severe | 4 | 61-100% |

Macrophage Analysis Through Immunofluorescent Staining: Explanted colons were analyzed through immunofluorescent staining of macrophages. Slides were deparaffinized through a series of xylene and ethanol washes followed by a water wash. Antigen retrieval of tissue sections was facilitated by citrate antigen retrieval buffer (10 mM citrate, pH 6.0) on high in the microwave for 15 minutes and allowed to cool in copper sulfate buffer (10 mM CuSO₄, 50 mM ammonium acetate). Blocking solution (Pierce Protein-free Blocking Buffer, 4% Goat serum, 2% BSA, 0.1% Triton, 0.1% Tween; Pierce Protein-free Blocking Buffer) was incubated on slides for 10 minutes respectively. The tissue was then microwaved with primary antibody (goat anti-CD206 1:100; rabbit anti-TNFα 1:100; mouse anti CD68 1:100) for 3 minutes on mid-low setting and incubated for 3 minutes followed by washing with TBS-T. Secondary antibody (rabbit anti-goat HRP 1:100; goat anti-rabbit HRP 1:100; goat anti-mouse HRP 1:100) was microwaved with tissue for 3 minutes on mid-low setting and incubated for 3 minutes followed by washing. Opal reagent (green, red, and blue 1:100 in appropriate diluent) were incubated with tissue for 10 minutes in the dark followed by washing. Antigen retrieval and antibody incubations were repeated for each stain. DRAQ5 was used as a nuclear marker. Tissue sections were then mounted with anti-fade reagent and imaged.

### Results—In-Life Observations

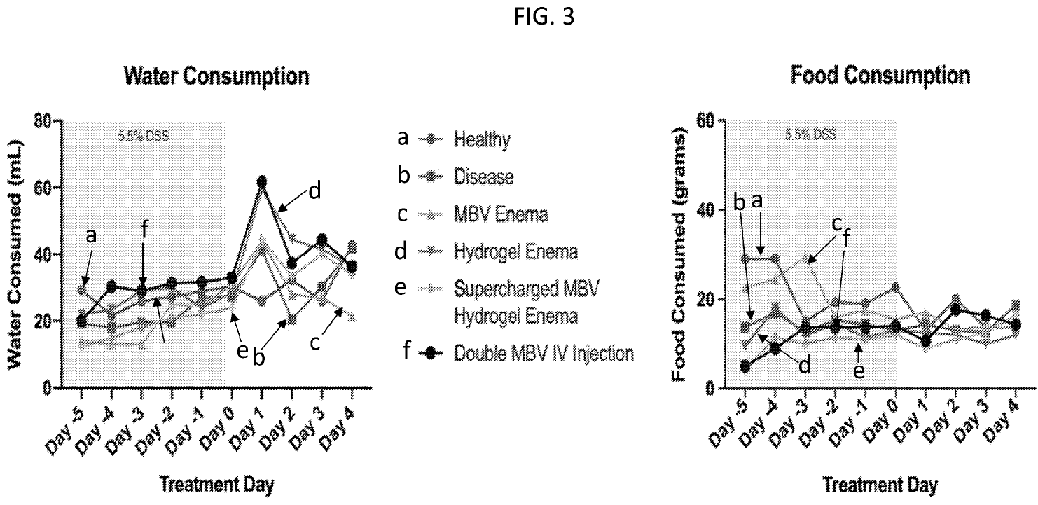

Food and Water Consumption: The food and water consumed by each rat was measured daily from Day −5 through Day 4 (FIG. 3). Water intake appeared to increase in groups undergoing disease induction on Day 1 after DSS water was removed, and normal water was given. Overall, food consumption remained stable throughout the study.

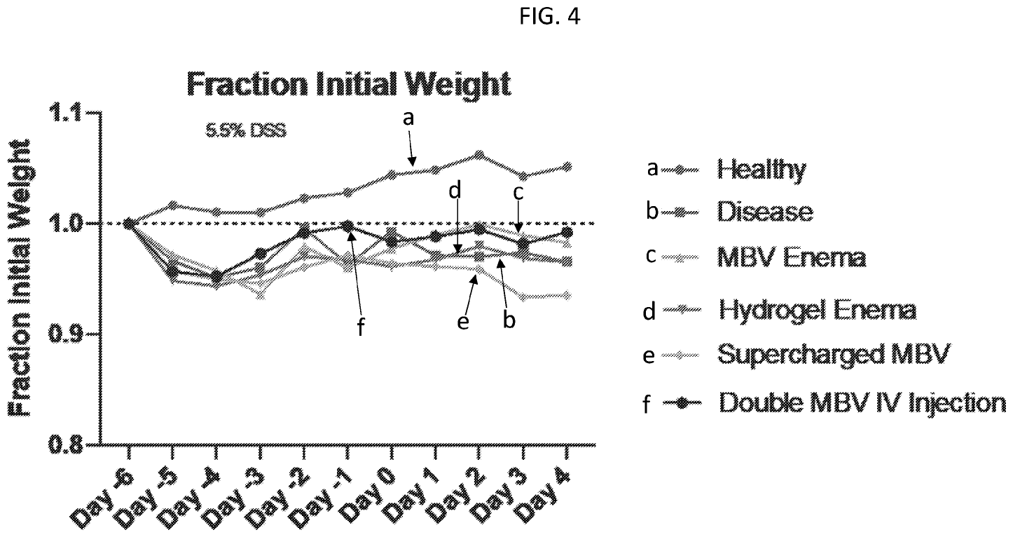

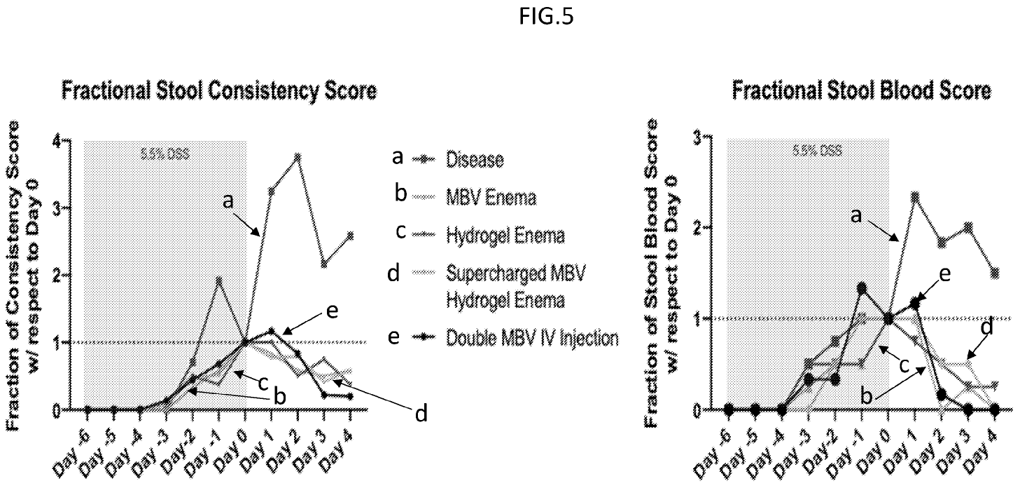

Weight Changes: Rats were weighed once daily from Day-6 through Day 4. Individual rat weights were normalized to their starting weight on Day-6 (FIG. 4). There appeared to be an overall loss in weight during the first three days the rats were exposed to DSS. Overall, weights appeared to stabilize once DSS water was removed, and treatments began.

Stool Consistency and Blood Content: Stool consistency and blood scores were determined daily for each rat. In the scoring system of both metrics, a higher score indicated higher levels of disease. Individual rat scores of stool consistency and blood content were normalized to the score given at Day 0 to determine changes from the end of disease induction to the end of treatment (FIG. 5). All treatment groups appeared to demonstrate a mitigation of disease progression.

### Clinical and Histological Outcomes

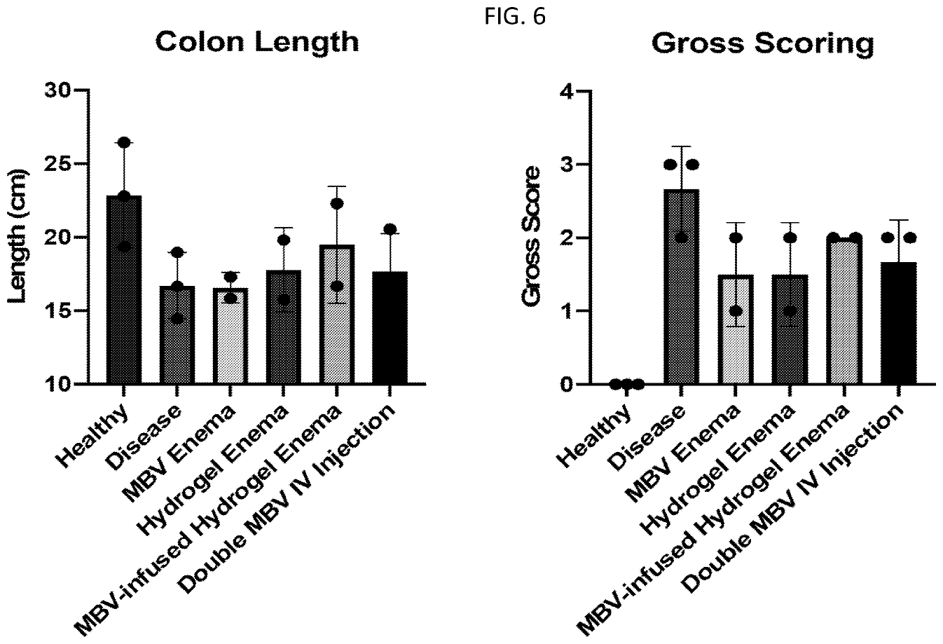

After explanting, the length of the colon from the cecum to the rectum was measured as an indicator of disease activity with a shorter colon corresponding to a more severe disease state. Overall, all of the animals given DSS appeared to have a shorter colon than the healthy control as expected. The colons were examined macroscopically for evidence of hyperemia, inflammation and ulceration. Overall, animals given treatment appeared to have lower gross scores than animals without treatment, as shown in FIG. 6.

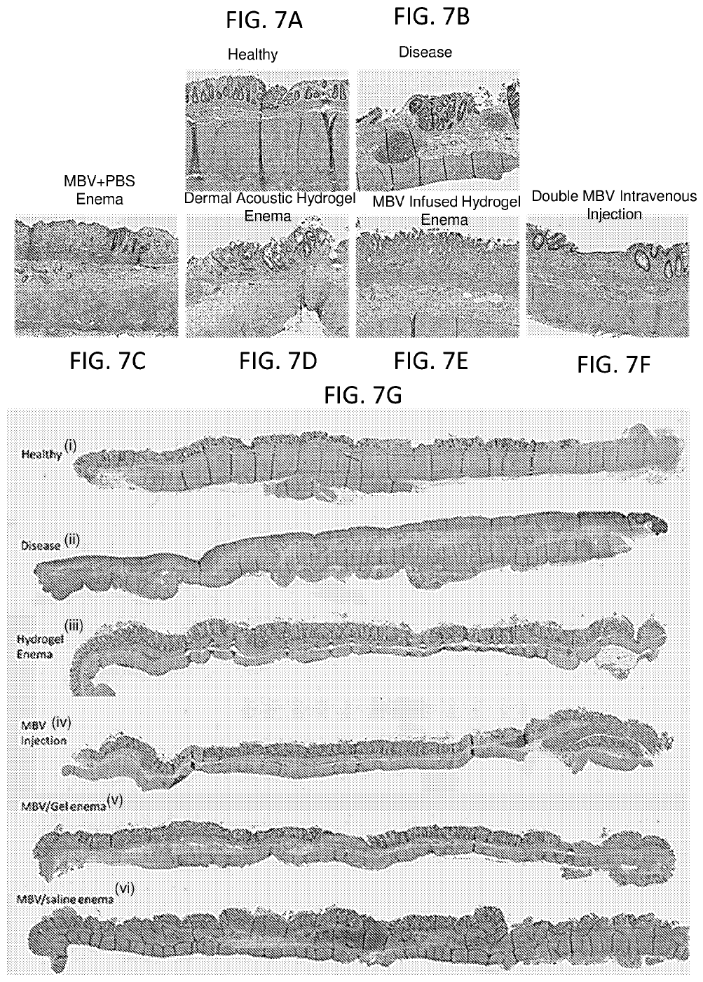

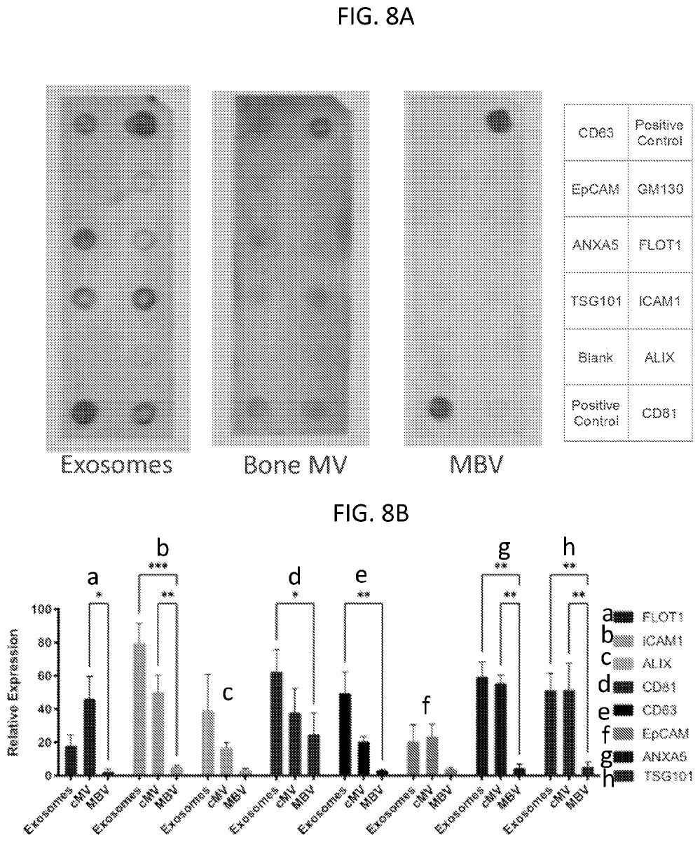

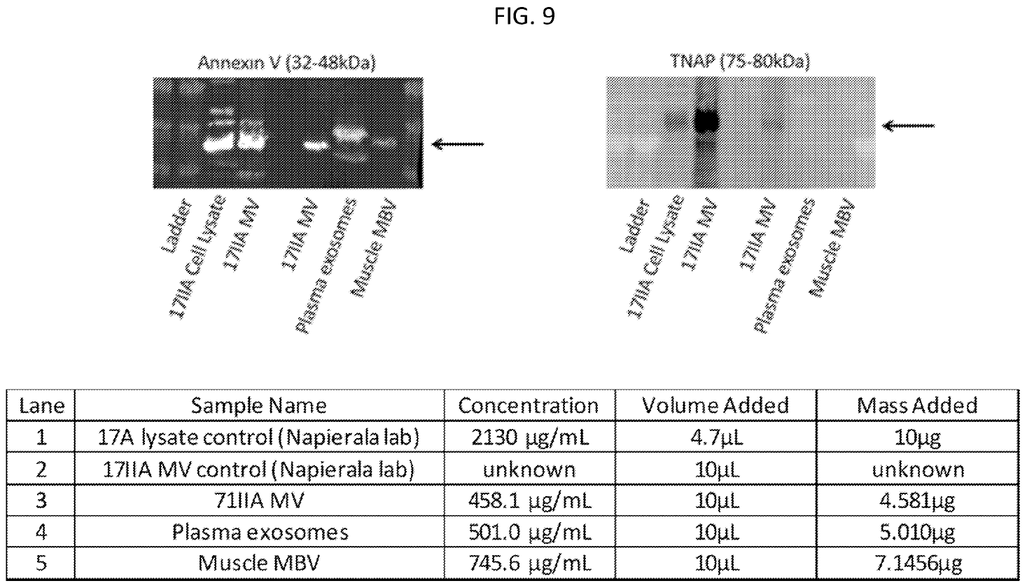

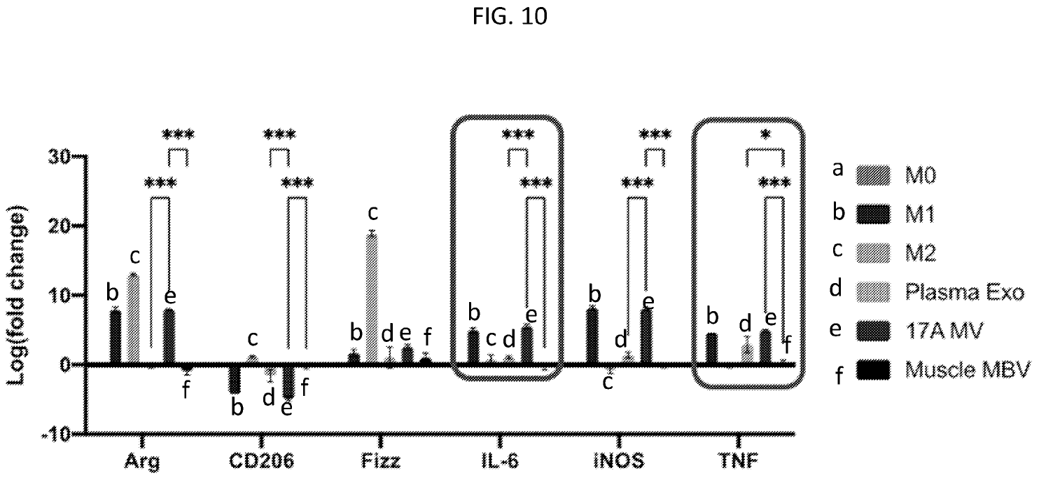

A histological analysis is shown in FIGS. 7A-7G and is discussed in detail below.

Normal healthy colon tissue: The colon tissue shown in FIG. 7A and FIG. 7G (i) was harvested from a healthy animal and is characterized by a uniformly thick mucosa with a confluent layer of epithelial cells. The lamina propria has normal cellularity, and there is a well-defined underlying muscularis mucosa. The submucosa contains a normal number of mononuclear cells, most of which are quiescent macrophages. The deepest layer is the thick, muscular layer referred to as the muscularis externa. This tissue section is representative of normal healthy colon tissue.

Diseased colon: The colon tissue shown in FIG. 7B and FIG. 7G (ii) was harvested from an animal treated with 5.5% DSS for 6 days and which subsequently received no therapeutic intervention. The morphology of the mucosa was markedly disrupted. Widely scattered, small clusters of mucosal epithelial cells were present as evidence of an attempt to regenerate the mucosal epithelium and restore the normal barrier function of the mucosal layer. There is an absence of any confluent layer of epithelial cells unlike the normal, healthy colon. The lamina propria has increased cellularity, which is indicative of the host inflammatory response to the tissue injury and the invasion of toxic colonic contents. There is marked disruption of the muscularis mucosa layer which normally separates the mucosa from the underlying submucosa. The cellularity in both the lamina propria and the submucosa is markedly increased, and two nodules of lymphoid cell accumulation can be seen within the submucosal layer of the diseased colon (dark circular areas). The muscularis externa is normal (as it is for all specimens).

ECM hydrogel treated group: Colonic tissue shown in FIG. 7D and FIG. 7G (iii) was harvested from an animal treated with ECM acoustic hydrogel made from dermal ECM. There is partial restitution of the colonic mucosa as evidenced by an increased number of mucosal epithelial cells compared to the diseased colon group. The cellularity of the lamina propria and the submucosa is less than that of the diseased group but not as low as the normal colon group. There is a clearly definable and intact muscularis mucosa. This histologic appearance represents partial healing of the colonic mucosa.

MBV+Saline treated group: The histologic appearance of colon tissue from this group, as shown in FIG. 7C and FIG. 7G (vi), was very similar to the ECM hydrogel treated group. There was partial restitution of the colonic mucosa, maintenance of an intact muscularis mucosa, and a submucosa which contained a slightly increased number of macrophages.

ECM hydrogel+MBV treated group: Exemplary tissue from this group is shown in FIG. 7E and FIG. 7G (v) and exhibits a remarkable and almost complete replacement of the mucosa, muscularis mucosa and submucosa. There was clear distinction from the diseased colon group. There was almost complete replacement of the mucosal epithelial cell population, and near normal cellularity in the lamina propria and submucosa. The muscularis mucosa is intact. Surprisingly and unexpectedly, the histological specimen from the ECM hydrogel+MBV group was nearly indistinguishable from the normal, healthy colonic mucosa indicating that the combination of ECM hydrogel+MBV had a synergistic impact on healing of the colonic tissue that was not seen with the ECM hydrogel or MBV alone, either as an enema or intravenous administration.

Summary: The group that received no ECM in any way elicited a marked destructive outcome with only minimal restorative attempts at colonic mucosal replacement. All MBV groups showed partial replacement of the colonic mucosa, contrasted with the minimal if any replacement of the disease only group. The combination of ECM hydrogel and MBV induced near complete restoration of the colonic mucosa. The combination of IV MBV plus ECM hydrogel markedly surpassed the results of all other MBV groups.

### Further Histological Evaluation

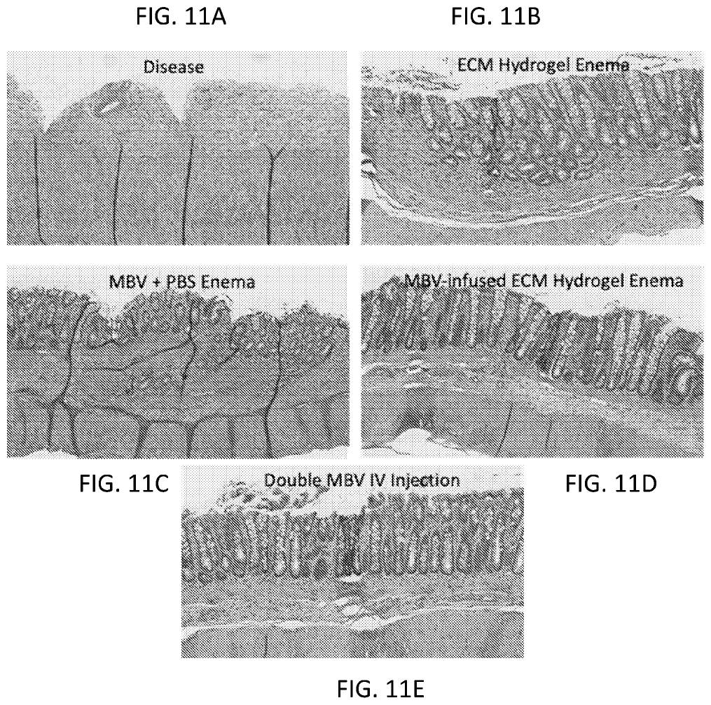

Histological samples of the above treated tissues were microscopically evaluated at higher magnifications and visual observation showed less inflammation in treated samples than disease samples. Treated animals qualitatively appeared to have healthier mucosa in relation to crypt presence and organization. Through visual observation of hematoxylin and eosin-stained samples, the combination of ECM hydrogel and MBV treated samples (FIG. 11D) presented with a greater healthy mucosa and submucosa indicated by well-organized intestinal crypts and minimal cellular infiltration. In diseased samples (FIG. 11A), intestinal crypts were completely gone in many areas whereas in treated samples (FIGS. 11B-11E), cells appeared to begin reorganizing to the commonly seen orientation and structure in healthy (FIG. 7A) colonic mucosa and submucosa.

Further, as demonstrated in FIG. 11D, the effect of local administration of MBV-infused ECM hydrogel to the rat colon resulted in greater mucosal and submucosal health as demonstrated by the histology, than administration of MBV+PBS locally (FIG. 11C) or ECM hydrogel alone (FIG. 11B) locally, suggesting that the combination may have a synergistic effect in healing the colon.

### MBV Treatments Show a Modulation of Macrophages Towards an M2 or Pro-Remodeling Phenotype

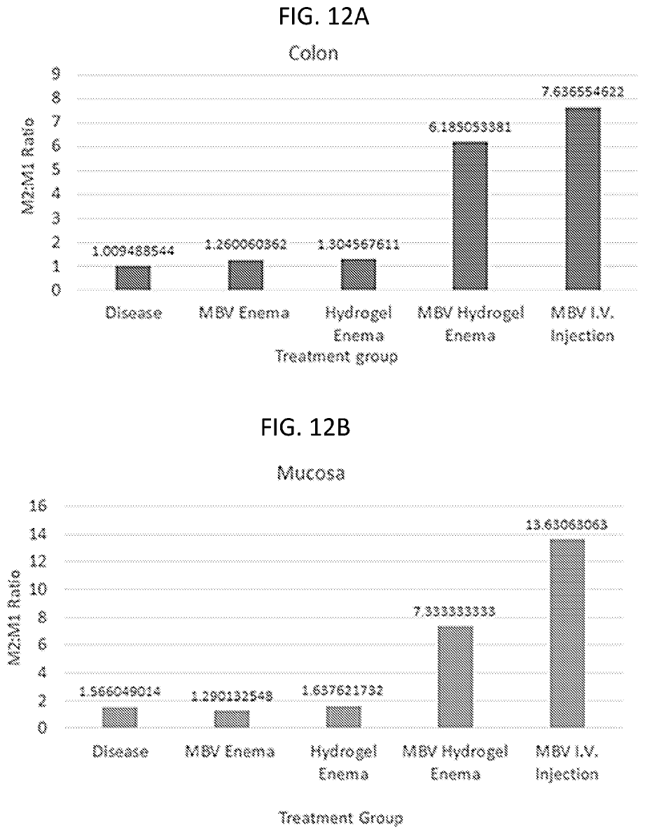

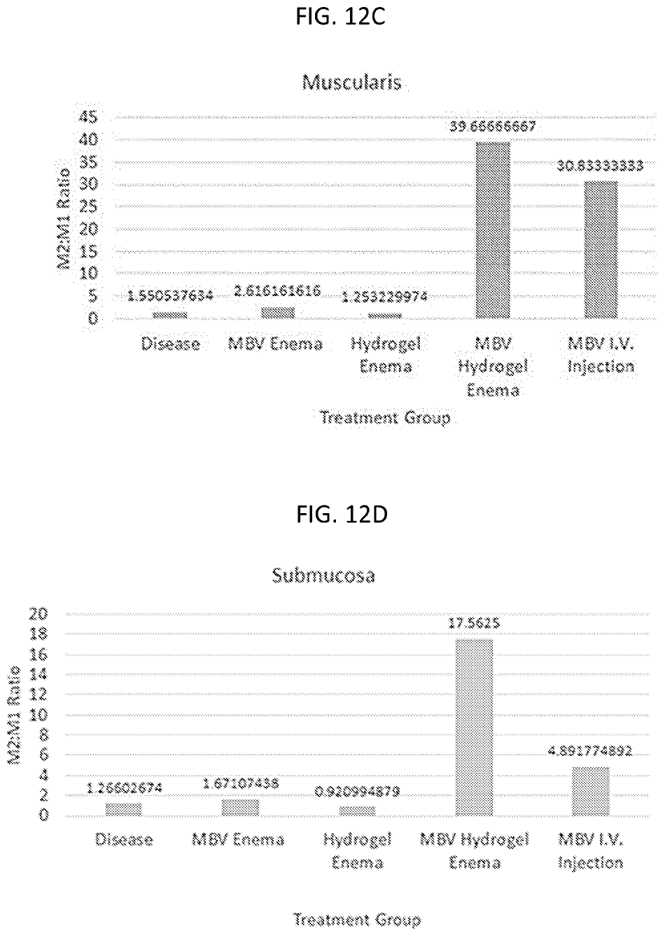

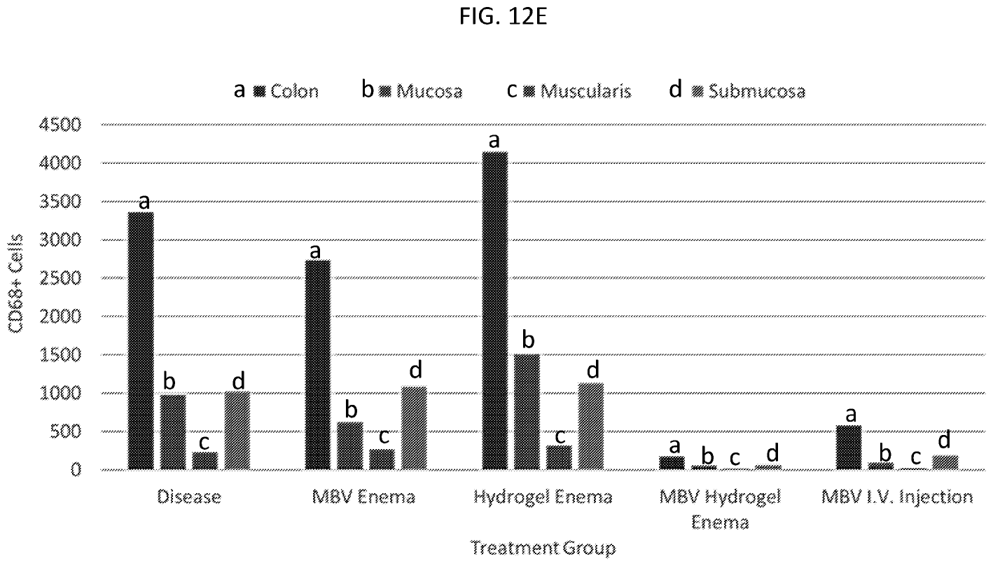

MBV previously have been shown to modulate macrophages towards a pro-healing, anti-inflammatory phenotype. This was further demonstrated in the treatment of DSS-induced ulcerative colitis. The M2: M1 macrophage ratio throughout various tissue layers from the treated rats is further demonstrated in FIGS. 12A-12E. The data show that systemic MBV administration or the combination ECM hydrogel-MBV enema promoted a dramatically higher M2: M1 ratio in colonic tissue as compared to ECM hydrogel enema alone or MBV enema alone. The M2: M1 ratio in colonic tissue treated with MBV hydrogel enema was greater than the effects on colonic tissue treated with MBV enema or hydrogel enema alone, even when added together, suggesting that the combination of MBV and hydrogel has a synergistic effect on modulation of macrophages towards an M2 phenotype in colonic tissue, and therefore on the M2: M1 macrophage ratios. In addition to counting M2 and M1 cells throughout the entire colon (FIG. 12A), the number of M2 (CD68+, CD206+) or M1 (CD68+, TNFα+) macrophages within the various histological layers of the colonic tissue were also counted (i.e. mucosa, muscularis, submucosa, see FIGS. 12B, FIG. 12C, and FIG. 12D, respectively). Overall, the data show that adding MBV to ECM hydrogel yields a synergistic, rather than just additive, effect on the M2: M1 ratio. Additionally, MBV administered either systemically or in an ECM hydrogel dramatically reduced the number of CD68+ cells (FIG. 12E), indicating a reduction in the total number of macrophages in those tissues.

### Summary

As demonstrated above, disease progression during the study was tracked through in-life observations including water consumption, food consumption, weight, stool consistency, and stool blood presence. With decreases in weight, increases in stool consistency scores, and presence of blood in stool, it was confirmed that stimulation of DSS-induced ulcerative colitis in test animals was successful.

Overall, the data show that ECM hydrogel with added exogenous MBV provides an increased rate of healing in the DSS-induced Ulcerative Colitis (UC) rat model based on in-life observations, histological analyses, and macrophage polarization analysis. In-life observations showed faster recovery in all treatment groups compared to the disease control. Gross scoring and histology confirmed the in-life observations showing a lower severity of disease in the colon macroscopically as well as microscopically. The organization and density of the mucosa and submucosa in treatment groups showed indication for the increased rate of healing with lower levels of inflammation and higher levels of mucosal organization through intestinal crypt presence. Macrophages in treated groups showed a greater shift towards a remodeling phenotype than the disease group only, further confirming the ability and result of an increased rate of healing due to treatments.

The results of the above studies suggest that MBV-infused ECM hydrogels have potent therapeutic potential to treat inflammatory diseases of the bowel, e.g., Inflammatory Bowel Disease such as Crohn's disease and Ulcerative Colitis.

In view of the many possible aspects to which the principles of our invention may be applied, it should be recognized that illustrated aspects are only examples of the invention and should not be considered a limitation on the scope of the invention. Rather, the scope of the invention is defined by the following claims. We therefore claim as our invention all that comes within the scope and spirit of these claims.

1. A composition comprising an extracellular matrix (ECM) hydrogel comprising:
a) solubilized extracellular matrix (ECM); and
b) exogenous matrix bound nanovesicles (MBV) derived from extracellular matrix, wherein the MBV do not express CD63 and CD81 or are CD63^{lo}CD81^{lo} and wherein the MBV do not contain alkaline phosphatase,
wherein the exogenous MBV are present in the ECM hydrogel at a concentration of at least about 1×10⁵ to 1×10²⁰ particles/mL, and wherein the composition:
i) is sheer thinning;
ii) has a storage modulus (G′) of about 50 Pa to about 200 Pa, and a loss modulus (G″) of about 5 Pa to about 20 Pa, and a G′ to G″ ratio of about 4:1 to about 15:1 at 37° C., and
iii) has a 50% degradation rate of 24 hours to 14 days.

2. The composition of claim 1, wherein the ECM hydrogel is an acoustic hydrogel or an enzymatic hydrogel.

3. (canceled)

4. A composition comprising:
an acidic solution comprising an exogenous acid
protease and solubilized extracellular matrix; and
exogenous MBV derived from extracellular matrix, wherein the MBV do not express CD63 and CD81 or are CD63^{lo}CD81^{lo} and wherein the MBV do not contain alkaline phosphatase,
wherein the exogenous MBV are present in the composition at a concentration of at least about 1×10⁵ to about 1×10²⁰ particles/mL,
wherein the acidic solution, when neutralized to a pH of between about 7.0 to about 7.8 forms a gel at a temperature greater than 25° C.

5. A composition comprising:
solubilized, extracellular matrix;
an inactivated exogenous acid protease;
exogenous MBV derived from extracellular matrix, wherein the MBV do not express CD63 and CD81 or are CD63^{lo}CD81^{lo} and wherein the MBV do not contain alkaline phosphatase,
wherein the exogenous MBV are present in the composition at a concentration of at least about 1×10⁵ to 1×10²⁰ particles/mL,
wherein the composition enters the liquid phase at a temperature less than 25° C. and enters a gel phase at a temperature greater than 25° C.;
wherein the composition has a pH of between about 7 and about 7.8.

6. The composition of claim 1, further comprising comminuted ECM.

7. The composition of claim 1, wherein the exogenous MBV are present in the composition at a concentration of about 1×10⁶ to 1×10²² particles/mL.

8. The composition of claim 1, wherein the solubilized ECM is present in the ECM hydrogel in an amount of a 1 mg/mL to 500 mg/ml; b) 1 mg/mL to 500 mg/mL; or c) 5 mg/mL to 50 mg/mL.

9. (canceled)

10. (canceled)

11. (canceled)

12. The composition of claim 1, wherein the ECM hydrogel and/or the MBV are derived from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus.

13. The composition of claim 1, wherein the MBV are not derived from bone or cardiac ECM.

14. The composition of claim 1, wherein the MBV are derived from extracellular matrix of urinary bladder, small intestine, dermis, liver, kidney, uterus, brain, blood vessel, lung, muscle, pancreas, placenta, stomach, spleen, colon, adipose tissue, or esophagus.

15. (canceled)

16. (canceled)

17. (canceled)

18. (canceled)

19. The composition of claim 4, wherein the acid protease is pepsin or trypsin, or a combination thereof.

20. (canceled)

21. The composition of claim 5, wherein the composition has a pH in the range of 7.2 to 7.4.

22. (canceled)

23. (canceled)

24. (canceled)

25. A method for treating a subject with inflammatory bowel disease comprising:
administering of the subject an effective amount of the composition claim 1, thereby treating the inflammatory bowel disease in the subject.

26. The method of claim 25, wherein the composition is administered to the subject's bowl by enema, or wherein the composition is administered orally.

27. (canceled)

28. The method of claim 25, wherein the method reduces inflammation in the subject's colon.

29. The method of claim 25, wherein the subject has ulcerative colitis.

30. (canceled)

31. (canceled)

32. (canceled)

33. (canceled)

34. (canceled)

35. A method for treating a subject with esophageal inflammation comprising:
administering to the subject's esophagus an effective amount of the composition of claim 1, thereby treating the esophageal inflammation in the subject.

36. The method of claim 35, where the composition is administered topically to the esophagus.

37. (canceled)

38. The method of claim 25, wherein administering the composition to the subject increases the number of M2 macrophages compared to M1 macrophages in subject's bowel.

39. (canceled)

40. The method of any claim 35, wherein administering the composition to the subject increases the number of M2 macrophages compares to M1 macrophages in the subject's esophagus.


SEQUENCE LISTING (3 sequences)

SEQ ID NO: 1 - RNA, 88 nt, Homo sapiens
  source 1..88: mol_type = other RNA
caccttgtcctcacggtccagttttcccaggaatcccttagatgctaagatggggattcctggaaatactgttcttgaggtcatggtt

SEQ ID NO: 2 - RNA, 110 nt, Homo sapiens
  source 1..110: mol_type = other RNA
agaagggctatcaggccagccttcagaggactccaaggaacattcaacgctgtcggtgagtttgggatttgaaaaaaccactgaccgttgactgtaccttggggtcctta

SEQ ID NO: 3 - RNA, 106 nt, Homo sapiens
  source 1..106: mol_type = other RNA
gcgcagcgccctgtctcccagcctgaggtgcagtgctgcatctctggtcagttgggagtctgagatgaagcactgtagctcaggaagagagaagttgttctgcagc